(19) United States
(12) Patent Application Publication
(10) Pub. No.: US 2024/0228562 A1
(43) Pub. Date: Jul. 11, 2024

(54) NOVEL LIPOCALIN MUTEINS SPECIFIC FOR CONNECTIVE TISSUE GROWTH FACTOR (CTGF)

(71) Applicant: PIERIS PHARMACEUTICALS GMBH, Hallbergmoos (DE)

(72) Inventors: Marina PAVLIDOU, Hallbergmoos (DE); Vanessa NEIENS, Hallbergmoos (DE); Eva-Maria HANSBAUER, Hallbergmoos (DE); Claudia WURZENBERGER, Hallbergmoos (DE); Thomas JAQUIN, Hallbergmoos (DE); Tanja HERRMANN, Hallbergmoos (DE); Stefan GRUENER, Hallbergmoos (DE); Gabriele MATSCHINER, Hallbergmoos (DE)

(21) Appl. No.: 18/554,273

(22) PCT Filed: Apr. 8, 2022
(86) PCT No.: PCT/EP2022/059406

§ 371 Date: Oct. 6, 2023

(30) Foreign Application Priority Data
Apr. 8, 2021 (EP) 21167385.0
Aug. 20, 2021 (EP) 21192311.5

(51) Int. Cl. C07K 14/47; A61K 38/00; A61P 11/00; G01N 33/68

(52) U.S. Cl. CPC C07K 14/4702; A61P 11/00; G01N 33/6887; A61K 38/00; C07K 2319/30; G01N 2333/475

(57) ABSTRACT

The present disclosure provides lipocalin muteins capable of binding CTGF as well as fusion proteins comprising said muteins, which are useful for analytical, diagnostic or therapeutical purposes, for example in the treatment of a fibrotic disease, a cancer, an autoimmune disease, or an infectious disease. The present disclosure also concerns methods of making the lipocalin muteins or fusion proteins described herein. The present disclosure further relates to nucleic acid molecules encoding such lipocalin muteins or fusion proteins. In addition, the present disclosure relates to therapeutic and/or diagnostic uses of such lipocalin muteins or fusion proteins as well as to compositions comprising one or more of such lipocalin muteins or fusion proteins.

### I. BACKGROUND

CTGF (UniProt P29279), also known as connective tissue growth factor or CCN2, is a member of the CCN family of proteins, a family of matricellular proteins associated to the extracellular matrix (ECM) involved in intercellular signaling (Ramazani et al., Matrix Biol. 68-69, 44-66 (2018), Holbourn et al., Trends Biochem. Sci. 33, 461-473 (2008)). The structure of CTGF is characteristic for members of the CCN family and comprises four functionally distinct domains: an insulin-like growth factor binding protein-like domain (IGFBP), a von Willebrand factor type C repeat domain (VWFC), a thrombospondin type-1 repeat (TSP type-1) and a cysteine-knot-containing domain (CTCK) (Holbourn et al., Trends Biochem. Sci. 33, 461-473 (2008)). While the insulin-like growth factor binding protein-like domain and the von Willebrand factor type C repeat form the N-terminal fragment, the thrombospondin type-1 repeat and a cysteine-knot-containing domain form the C-terminal fragment of CTGF. The hinge region between the N-terminal and the C-terminal fragment is subject to proteolytic cleavage by metalloproteases. The CTGF gene (6q23.2) comprises 5 exons and codes for a 349 amino acid protein. CTGF is highly conserved among vertebrates with 91% identity between human and mouse CTGF on the gene level and 95% identity on the protein level (Ramazani et al., Matrix Biol. 68-69, 44-66 (2018)).

CTGF is highly expressed at the embryonic stage mediating skeletal, cardiovascular or renal developmental processes. In adulthood, CTGF expression is rather low but is strongly induced upon certain stimuli such as cytokine or growth factor stimulation and mechanical stress (Kubota et al., Clin. Sci. 128, 181-196 (2014); Leask, J. Cell Commun. Signal. 7(3): 203-205 (2013)). CTGF expression is further described either on the mRNA or on the protein level in many tissues including smooth muscles, thyroid, spleen, kidney, prostate, endometrium, cerebral cortex, lymph nodes, lung, liver gastrointestinal tract and skin (Uhlen et al., Science 347(6220):1260419 (2015)). CTGF was initially described to be expressed in endothelial cells and fibroblasts where it was associated with tissue regeneration wound healing and angiogenesis (Bradham et al., J. Cell Biol. 114, 1285-1294 (1991); Igarashi et al., Mol. Biol. Cell 4, 637-645 (1993)). CTGF plays an important role in several biological processes such as cell adhesion, extracellular matrix remodeling, skeletal development, chondrogenesis, angiogenesis, wound healing and proliferation. The function of CTGF which includes activation of signaling pathways, regulation of matrix turnover, cytokine and growth factor regulation, is dependent on the respective cellular context and interacting proteins.

A variety of proteins such as receptors, cytokines and ECM proteins have been described to interact with CTGF. Cell surface receptors interacting with CTGF are integrins (e.g., α5β3, α1β3, α5β1), heparan sulfate proteoglycans (e.g., syndecan 4), lipoprotein receptor related proteins (e.g., LRP1, LRP6) and tyrosine kinase receptors (e.g., TK receptor A) (Lau, J. Cell Commun. Signal. 10, 121-127 (2016)). Among the different cytokines and growth factors interacting with CTGF transforming growth factor β (TGF-β) plays a crucial role in the development of fibrotic diseases as described below (Abreu et al., Nat. Cell Biol. 4, 599-604 (2002)). CTGF further binds to other cytokines and growth factors such as vascular endothelial growth factors (VEGFs), fibroblast growth factor 2 (FGF-2), bone morphogenic protein 4 (BMP4), platelet-derived growth factor B (PDGFB) and others (Chen et al., Front. Cell Dev. Biol. 8, 1-17 (2020)). In order to mediate function in cell adhesion, motility and tissue remodeling CTGF interacts with components of the ECM such as fibronectin, aggrecan and heparan sulfate proteoglycans. In the context of cell adhesion, CTGF is seen as a bridging molecule for components of the ECM to integral cell surface proteins, and blocking of CTGF in vitro leads to inhibition of the attachment of cells on the surface.

The pathogenesis of fibrosis is considered a dysregulated wound healing process as a response to repetitive microinjuries in different organs such as lung or kidney (Wynn, J. Exp. Med. 208, 1339-1350 (2011)). CTGF is a main component in regulating the processes in the spectrum of wound healing to fibrosis through its interaction with several factors resulting in cell proliferation, differentiation, motility, adhesion and matrix turnover. Upon injury, the antifibrinolytic coagulation cascade and circulating platelets are activated by inflammatory mediators released from the damaged epithelium or endothelium. These activated platelets induce the recruitment of immune cells, such as macrophages, neutrophils, and T cells, secreting TGF-β and other cytokines which augment the inflammatory response and the activation, proliferation and migration of myofibroblasts. Myofibroblasts induce wound closure due to their contractile function and secrete ECM components mediating re-epithelialization and tissue reconstitution. Under normal conditions this process is ceased by the elimination of effector cells and ECM components. However, upon repetitive injury the repair process becomes dysregulated leading to an excessive deposition of ECM and irreversible fibrotic remodeling of the tissue. Myofibroblasts in idiopathic pulmonary fibrosis (IPF) appear to be resistant to apoptotic processes and instead of dying, these cells persist and facilitate further fibrogenesis, contributing to an excessive deposition of ECM (Shimbori et al., Curr. Opin. Pulm. Med. 19, 446-452 (2013)). TGF-β and CTGF are widely regarded as universal mediators of fibrogenesis, although the precise mechanisms that underlie their concerted effects remain unclear (A. Leask et al., J. Biol. Chem. 278, 13008-13015 (2003)).

CTGF is found to be overexpressed in fibrotic tissue of patients suffering from idiopathic pulmonary fibrosis (IPF), cardiac fibrosis, liver fibrosis and kidney fibrosis (Pan et al., Eur. Respir. J. 17, 1220-1227 (2001); Chen et al., Front. Cell Dev. Biol. 8, 1-17 (2020)). A role of CTGF expression has been also described in mouse models of bleomycin-induced pulmonary fibrosis and in a radiation-induced lung fibrosis model in rats (Ponticos et al., Arthritis Rheum. 60, 2142-2155 (2009); Wang et al, Fibrogenesis Tissue Repair 4; 4 (2011); Bickelhaupt, JNCI J. Natl. Cancer Inst. 109, 1-11 (2017)). Here, blocking of CTGF by a monoclonal antibody reversed the process of radiation-induced lung remodeling in rats (Bickelhaupt, JNCI J. Natl. Cancer Inst. 109, 1-11 (2017)).

In a phase 2 clinical trial, the anti-CTGF monoclonal antibody FG-3019/pamrevlumab attenuated disease progression in patients suffering from IPF demonstrating that targeting CTGF has the potential to be a therapeutic option for IPF (Richeldi et al., Lancet Respir. Med. 8, 25-33 (2020)). IPF is a devasting and fatal disease of unknown cause with median survival of 3-5 years after diagnosis (Lederer et al., N. Engl. J. Med. 378, 1811-1823 (2018)). Patients suffer from fibrotic remodeling of the lung architecture and excessive deposition of ECM leading to loss of lung elasticity, impairment of gas exchange and finally organ failure. Treatment options are limited and the only two FDA-approved drugs for IPF, nintedanib and pirfenidone, are capable though of slowing down the decline in lung function, but are due to gastrointestinal and other drug-related tolerability issues not well-tolerated by patients leading in many cases to the disruption of treatment (Galli et al., Respirology 22, 1171-1178 (2017)). Thus, a high medical need exists for alternative treatment options for IPF that efficiently attenuate the lung function decline while having an improved safety profile with less side effects as observed with current standard of care treatment.

Besides IPF, CTGF also plays a role in carcinogenesis and is depending on the interaction with other CCN proteins and molecules in the tumor microenvironment positively or negatively correlated with the development of tumors and metastasis (Shen et al., Trends in Cancer, doi:10.1016/j.trecan.2020.12.001 (2020)). CTGF is increasingly expressed in several types of cancer such as breast cancer, chondrosarcomas, enchondroma, glioma, pancreatic cancer, thyroid cancer, intrahepatic cholangiocarcinoma, neuroendocrine tumors and squamous cell carcinoma of the tongue. A clear role of CTGF in the formation of metastasis has been demonstrated by showing that melanoma cell lines lacking CTGF injected in mice fail to form metastasis in the lung (Hutchenreuther et al., J. Invest. Dermatol. 135, 2805-2813 (2015)) and by the anti-CTGF monoclonal antibody FG-3019 leading to inhibition of the migration of human melanoma cell lines (Finger et al., Oncogene 33, 1093-1100 (2014)).

CTGF is also involved in the pathology of ocular diseases such as diabetic retinopathy and glaucoma. A role of CTGF is also described in Duchenne muscular dystrophy and in systemic sclerosis, an autoimmune disease (Chen et al., Front. Cell Dev. Biol. 8, 1-17 (2020)).

CTGF could also play a role in the lung pathology of acute COVID-19 disease. Studies have shown that the pulmonary histopathology of serious COVID-19 disease is associated with diffuse alveolar damage (DAD), as well as severe damage to the existing pulmonary endothelium as well as fibrotic changes in lung tissue (Wang et al., JAMA—J Am Med Assoc. 323(11):1061-1069 (2020); Mo et al, Eur Respir J. 2020. doi:10.1183/13993003.01217-2020). CTGF could be directly involved in several of these processes based on its clear profibrotic function but also its implication in the regulation of endothelial cell function and angiogenesis (Brigstock, Angiogenesis 5, 153-165 (2002)). Moreover, the use of regenerative, antifibrotic agents for the treatment of acute COVID-19 disease could play an important role because patients with severe disease showed various signs of fibrotic tissue changes, from fibrosis-associated organizing pneumonia to acute lung damage as a precursor to fibrosis (Shi et al., Lancet Infect Dis. 20(4):425-434 (2020)).

The anti-CTGF antibody pamrevlumab is also currently being tested in two clinical trials in patients with acute COVID-19. In a phase 2 study, the effect of systemic administration of the antibody on the need for mechanical ventilation in hospitalized patients is investigated (NCT04432298). In a further phase 3 study, the effect on blood oxygenation and on the need for invasive mechanical ventilation of hospitalized patients is investigated (EudraCT Number: 2020-001472-14). In addition, a further phase 2 clinical trial in patients with signs of interstitial lung disease following acute COVID-19 disease is planned to investigate the long-term effect on further recovery from the lung tissue damage.

Due to the roles of CTGF as component of the extracellular matrix, there is a long-felt unmet need for compounds that bind to human CTGF and block CTGF-mediated responses, providing potential therapeutics for various diseases including fibrotic diseases and cancer. Furthermore, there is an unmet need for inhalable compounds that are used for the treatment of interstitial lung diseases such as pulmonary fibrosis. As CTGF is found highly expressed in the fibrotic lung there is a desire for CTGF-targeting compounds that are suitable for lung delivery via the inhaled route of administration.

### II. DEFINITIONS

The following list defines terms, phrases, and abbreviations used throughout the instant specification. All terms listed and defined herein are intended to encompass all grammatical forms.

As used herein, unless otherwise specified, “connective tissue growth factor” or “CTGF” means human CTGF (huCTGF). Human CTGF means a full-length protein defined by UniProt P29279 (version 197 of 10 Feb. 2021), a fragment thereof, or a variant thereof. Human CTGF is encoded by the CTGF gene. CTGF is also known as cellular communication network factor 2 (CCN2). In some particular embodiments, CTGF of non-human species, e.g. cynomolgus CTGF and mouse CTGF, is used.

As used herein, “binding affinity” describes the ability of a biomolecule (e.g., a polypeptide or a protein) of the disclosure (e.g., a lipocalin mutein, an antibody, a fusion protein, or any other peptide or protein) to bind a selected target (and form a complex). Binding affinity is measured by a number of methods known to those skilled in the art including, but not limited to, fluorescence titration, enzyme-linked immunosorbent assay (ELISA)-based assays, including direct and competitive ELISA, calorimetric methods, such as isothermal titration calorimetry (ITC), and surface plasmon resonance (SPR). These methods are well-established in the art and some examples of such methods are further described herein. Binding affinity is thereby reported as a value of dissociation constant (K_{D}), half maximal effective concentration (EC₅₀), or half maximal inhibitory concentration (IC₅₀) measured using such methods. A lower K_{D}, EC₅₀, or IC₅₀ value reflects better (higher) binding ability (affinity).

As used herein, the term “detect,” “detection,” “detectable,” or “detecting” is understood both on a quantitative and a qualitative level, as well as a combination thereof. It thus includes quantitative, semi-quantitative, and qualitative measurements performed on a biomolecule of the disclosure.

As used herein, “detectable affinity” generally means the binding ability between a biomolecule and its target, reported by a K_{D}, EC₅₀, or IC₅₀ value, is at most about 10⁻⁵ M or lower. A binding affinity, reported by a K_{D}, EC₅₀, or IC₅₀ value, higher than 10⁻⁵ M is generally no longer measurable with common methods such as ELISA and SPR and is therefore of secondary importance. Thus, “detectable affinity” may refer to a K_{D} value of about 10⁻⁵ M or lower as determined by ELISA or SPR, preferably SPR.

It is noted that the complex formation between a biomolecule of the disclosure and its target is influenced by many different factors such as the concentrations of the respective target, the presence of competitors, pH and the ionic strength of the buffer system used, the experimental method used for determination of the binding affinity (e.g., fluorescence titration, competitive ELISA (also called competition ELISA), and surface plasmon resonance), and even the mathematical algorithm used for evaluation of the experimental data.

Therefore, it is clear to the skilled person that binding affinity reported by a K_{D}, EC₅₀, or IC₅₀ value may vary within a certain experimental range, depending on the method and experimental setup. This means that there may be a slight deviation in the measured K_{D}, EC₅₀, or IC₅₀ values or a tolerance range depending, for example, on whether such values were determined by ELISA (including direct or competition ELISA), by SPR, or by another method.

As used herein, “specific for,” “specific binding,” “specifically bind,” or “binding specificity” relates to the ability of a biomolecule to discriminate between the desired target (for example, CTGF) and one or more reference targets (for example, human neutrophil gelatinase-associated lipocalin). It is understood that such specificity is not an absolute but a relative property and can be determined, for example, by means of SPR, western blots, ELISA, fluorescence activated cell sorting (FACS), radioimmunoassay (RIA), electrochemiluminescence (ECL), immunoradiometric assay (IRMA), ImmunoHistoChemistry (IHC), and peptide scans.

When used herein in the context of the lipocalin muteins of the present disclosure that bind to CTGF, the term “specific for,” “specific binding,” “specifically bind,” or “binding specificity” means that the lipocalin mutein binds to, reacts with, or is directed against CTGF, as described herein, but does not essentially bind another protein. The term “another protein” includes any proteins that are not CTGF or proteins closely related to or being homologous to CTGF. However, CTGF from species other than human and fragments and/or variants of CTGF are not excluded by the term “another protein.” The term “does not essentially bind” means that the lipocalin muteins of the present disclosure bind another protein with lower binding affinity than CTGF, i.e., show a cross-reactivity of less than 30%, preferably 20%, more preferably 10%, particularly preferably less than 9, 8, 7, 6, or 5%. Whether the lipocalin mutein specifically reacts as defined herein above can easily be tested, inter alia, by comparing the reaction of a lipocalin mutein of the present disclosure with CTGF and the reaction of said lipocalin with (an)other protein(s).

As used herein, the term “lipocalin” refers to a monomeric protein of approximately 18-20 kDa in weight, having a cylindrical βpleated sheet supersecondary-structural region comprising a plurality of β-strands (preferably eight β-strands designated A to H) connected pair-wise by a plurality of (preferably four) loops at one end to thereby comprise a ligand-binding pocket and define the entrance to the ligand-binding pocket. Preferably, the loops comprising the ligand-binding pocket used in the present disclosure are loops connecting the open ends of β-strands A and B, C and D, E and F, and G and H, and are designated loops AB, CD, EF, and GH. It is well-established that the diversity of the said loops in the otherwise rigid lipocalin scaffold gives rise to a variety of different binding modes among the lipocalin family members, each capable of accommodating targets of different size, shape, and chemical character (reviewed, e.g., in Skerra, Biochim Biophys Acta, 1482, 337-50 (2000), Flower et al., Biochim Biophys Acta, 1482, 9-24 (2000), Flower, Biochem J, 318 (Pt 1), 1-14 (1996)). It is understood that the lipocalin family of proteins has naturally evolved to bind a wide spectrum of ligands, sharing unusually low levels of overall sequence conservation (often with sequence identities of less than 20%) yet retaining a highly conserved overall folding pattern. The correspondence between positions in various lipocalins is also well-known to one of skill in the art (see, e.g., U.S. Pat. No. 7,250,297). Proteins falling in the definition of “lipocalin” as used herein include, but are not limited to, tear lipocalin, Lipocalin-2 or neutrophil gelatinase-associated lipocalin, apolipoprotein D, and Von Ebner's gland protein.

As used herein, unless otherwise specified, “Lipocalin-2” or “neutrophil gelatinase-associated lipocalin” refers to human Lipocalin-2 (hLcn2) or human neutrophil gelatinase-associated lipocalin (hNGAL) and further refers to mature human Lipocalin-2 or mature human neutrophil gelatinase-associated lipocalin. The term “mature” when used to characterize a protein means a protein essentially free from the signal peptide. A “mature hNGAL” of the instant disclosure refers to the mature form of human neutrophil gelatinase-associated lipocalin, which is free from the signal peptide. Mature hNGAL is described by residues 21-198 of the sequence deposited with the SWISS-PROT Data Bank under Accession Number P80188, the amino acid sequence of which is indicated in SEQ ID NO: 1.

As used herein, a “native sequence” refers to a protein or a polypeptide having a sequence that occurs in nature or having a wild-type sequence, regardless of its mode of preparation. Such native sequence protein or polypeptide can be isolated from nature or can be produced by other means, such as by recombinant or synthetic methods.

The “native sequence lipocalin” refers to a lipocalin having the same amino acid sequence as the corresponding polypeptide derived from nature. Thus, a native sequence lipocalin can have the amino acid sequence of the respective naturally-occurring (wild-type) lipocalin from any organism, in particular, a mammal. The term “native sequence”, when used in the context of a lipocalin specifically encompasses naturally-occurring truncated or secreted forms of the lipocalin, naturally-occurring variant forms such as alternatively spliced forms and naturally-occurring allelic variants of the lipocalin. The terms “native sequence lipocalin” and “wild-type lipocalin” are used interchangeably herein.

As used herein, a “mutein,” a “mutated” entity (whether protein or nucleic acid), or “mutant” refers to the exchange, deletion, or insertion of one or more amino acids or nucleotides, compared to the naturally-occurring (wild-type) protein or nucleic acid. Said term also includes fragments of a mutein as described herein. The present disclosure explicitly encompasses lipocalin muteins, as described herein, having a cylindrical βpleated sheet supersecondary structural region comprising eight βstrands connected-pair-wise-by four loops at one end to thereby comprise a ligand-binding pocket and define the entrance of the ligand-binding pocket, wherein at least one amino acid of each of at least three of said four loops has been mutated as compared to native sequence lipocalin. Lipocalin muteins of the present disclosure preferably have the function of binding CTGF as described herein.

As used herein, the term “fragment,” in connection with the lipocalin muteins of the disclosure, refers to proteins or polypeptides derived from full-length mature hNGAL or lipocalin muteins that are N-terminally and/or C-terminally truncated, i.e., lacking at least one of the N-terminal and/or C-terminal amino acids. Such fragments may include at least 10 or more, such as 20 or 30 or more, consecutive amino acids of the primary sequence of mature hNGAL or the lipocalin mutein it is derived from and are usually detectable in an immunoassay of mature hNGAL. Such a fragment may lack up to 2, up to 3, up to 4, up to 5, up to 10, up to 15, up to 20, up to 25, or up to 30 (including all numbers in between) of the N-terminal and/or C-terminal amino acids. It is understood that the fragment is preferably a functional fragment of mature hNGAL or the lipocalin mutein from which it is derived, which means that it preferably retains the binding specificity, preferably to CTGF, of mature hNGAL or the lipocalin mutein it is derived from. As an illustrative example, such a functional fragment may comprise at least amino acids at positions 28-136, preferably at least amino acids at positions 13-157, corresponding to the linear polypeptide sequence of mature hNGAL.

A “fragment” with respect to the corresponding target CTGF of a lipocalin mutein of the disclosure, refers to N-terminally and/or C-terminally truncated CTGF or protein domains of CTGF. Fragments of CTGF as described herein retain the capability of the full-length CTGF to be recognized and/or bound by a lipocalin mutein of the disclosure. As an illustrative example, the fragment may comprise, consist essentially of, or consist of one or more domains of CTGF. Such a domain may comprise amino acids of the domains of CTGF, such as the individual or combined amino acid sequences of domain 1 (IGFBP, residues 27-98 of UniProt Protein ID P29279), domain 2 (VWFC, residues 101-167), domain 3 (TSP type-1, 198-243) and domain 4 (CTCK, residues 256-330).

As used herein, the term “variant” relates to derivatives of a protein or polypeptide that include mutations, for example by substitutions, deletions, insertions, and/or chemical modifications of an amino acid sequence or nucleotide sequence. In some embodiments, such mutations and/or chemical modifications do not reduce the functionality of the protein or peptide. Such substitutions may be conservative, i.e., an amino acid residue is replaced with a chemically similar amino acid residue. Examples of conservative substitutions are the replacements among the members of the following groups: 1) alanine, serine, and threonine; 2) aspartic acid and glutamic acid; 3) asparagine and glutamine; 4) arginine and lysine; 5) isoleucine, leucine, methionine, and valine; and 6) phenylalanine, tyrosine, and tryptophan. Such variants include proteins or polypeptides, wherein one or more amino acids have been substituted by their respective D-stereoisomers or by amino acids other than the naturally occurring 20 amino acids, such as, for example, ornithine, hydroxyproline, citrulline, homoserine, hydroxylysine, norvaline. Such variants also include, for instance, proteins or polypeptides in which one or more amino acid residues are added or deleted at the N- and/or C-terminus. Generally, a variant has at least about 50%, 60%, 70%, 75%, 80%, 85%, 90%, 92%, 95% or at least about 98% amino acid sequence identity with the native sequence protein or polypeptide. A variant preferably retains the biological activity, e.g., binding the same target, of the protein or polypeptide it is derived from.

The term “variant”, as used herein with respect to the corresponding protein ligand CTGF of a lipocalin mutein of the disclosure, relates to CTGF or fragment thereof, respectively, that has one or more, such as 1, 2, 3, 4, 5, 6, 7, 8, 9, 10, 12, 14, 16, 18, 20, 22, 24, 26, 28, 30, 40, 50, 60, 70, 80 or more, amino acid substitutions, deletions and/or insertions in comparison to the native sequence of CTGF (wild-type CTGF), such as CTGF as deposited with UniProt Protein ID P29279 as described herein. A CTGF variant, respectively, has preferably an amino acid sequence identity of at least 50%, 60%, 70%, 80%, 85%, 90% or 95% with a wild-type CTGF, such as CTGF as deposited with UniProt Protein ID P29279 as described herein. A CTGF variant as described herein retains the ability to bind lipocalin muteins specific to CTGF disclosed herein.

The term “variant”, as used herein with respect to a lipocalin mutein, relates to a lipocalin mutein or fragment thereof of the disclosure, wherein the sequence has mutations, including substitutions, deletions, and insertions, and/or chemical modifications. A variant of lipocalin mutein as described herein retains the biological activity, e.g., binding to CTGF, of the lipocalin mutein from which it is derived. Generally, a lipocalin mutein variant has at least about 50%, 60%, 70%, 75%, 80%, 85%, 90%, 92%, 95%, 98% amino acid sequence identity with the lipocalin mutein from which it is derived.

As used herein, the term “mutagenesis” refers to the introduction of mutations into a polynucleotide or amino acid sequence. Mutations are preferably introduced under experimental conditions such that the amino acid naturally occurring at a given position of the protein or polypeptide sequence can be altered, for example substituted by at least one amino acid. The term “mutagenesis” also includes the (additional) modification of the length of sequence segments by deletion or insertion of one or more amino acids. Thus, it is within the scope of the disclosure that, for example, one amino acid at a chosen sequence position is replaced by a stretch of three amino acids, leading to an addition of two amino acid residues compared to the length of the respective segment of the native protein or polypeptide amino acid sequence. Such an insertion or deletion may be introduced independently from each other in any of the sequence segments that can be subjected to mutagenesis in the disclosure. In one exemplary embodiment of the disclosure, an insertion may be introduced into an amino acid sequence segment corresponding to the loop AB of the native sequence lipocalin (cf. International Patent Publication No. WO 2005/019256, which is incorporated by reference in its entirety herein).

As used herein, the term “random mutagenesis” means that no predetermined mutation (alteration of an amino acid) is present at a certain sequence position but that at least two amino acids can be incorporated with a certain probability at a predefined sequence position during mutagenesis.

As used herein, the term “sequence identity” or “identity” denotes a property of sequences that measures their similarity or relationship. The term “sequence identity” or “identity” as used in the present disclosure means the percentage of pair-wise identical residues—following (homologous) alignment of a sequence of a polypeptide of the disclosure with a sequence in question—with respect to the number of residues in the longer of these two sequences. Sequence identity is measured by dividing the number of identical amino acid residues by the total number of residues and multiplying the product by 100.

As used herein, the term “sequence homology” or “homology” has its usual meaning, and homologous amino acid includes identical amino acids as well as amino acids which are regarded to be conservative substitutions at equivalent positions in the linear amino acid sequence of a protein or a polypeptide of the disclosure (e.g., any lipocalin muteins of the disclosure).

A skilled artisan will recognize available computer programs, for example BLAST (Altschul et al., Nucleic Acids Res, 1997, 25, 3389-402), BLAST2 (Altschul et al., J Mol Biol, 1990, 215, 403-10), and Smith-Waterman (Smith and Waterman, J Mol Biol, 1981, 147, 195-7), for determining sequence homology or sequence identity using standard parameters. The percentage of sequence homology or sequence identity can, for example, be determined herein using the program BLASTP, version 2.2.5, Nov. 16, 2002 (Altschul et al., 1997). In this embodiment, the percentage of homology is based on the alignment of the entire protein or polypeptide sequences (matrix: BLOSUM 62; gap costs: 11.1; cutoff value set to 10⁻³) including the propeptide sequences, preferably using the wild-type protein scaffold as reference in a pairwise comparison. It is calculated as the percentage of numbers of “positives” (homologous amino acids) indicated as result in the BLASTP program output divided by the total number of amino acids selected by the program for the alignment.

Specifically, in order to determine whether the amino acid sequence of a lipocalin (mutein) is different from that of a wild-type lipocalin with regard to a certain position in the amino acid sequence of the wild-type lipocalin, a skilled artisan can use means and methods well-known in the art, e.g., alignments, either manually or by using computer programs such as BLAST 2.0, which stands for Basic Local Alignment Search Tool, or ClustalW, or any other suitable program which is suitable to generate sequence alignments. Accordingly, a wild-type sequence of lipocalin can serve as “subject sequence” or “reference sequence,” while the amino acid sequence of a lipocalin (mutein) different from the wild-type lipocalin described herein serves as “query sequence.” The terms “wild-type sequence,” “reference sequence,” and “subject sequence” are used interchangeably herein. A preferred wild-type sequence of a lipocalin is the sequence of hNGAL as shown in SEQ ID NO: 1.

“Gaps” are spaces in an alignment that are the result of additions or deletions of amino acids. Thus, two copies of exactly the same sequence have 100% identity, but sequences that are less highly conserved, and have deletions, additions, or replacements, may have a lower degree of sequence identity.

As used herein, the term “position” means the position of either an amino acid within an amino acid sequence disclosed herein or the position of a nucleotide within a nucleic acid sequence disclosed herein. It is to be understood that when the term “correspond” or “corresponding” is used herein in the context of the amino acid sequence positions of one or more lipocalin muteins, a corresponding position is not only determined by the number of the preceding nucleotides or amino acids. Accordingly, the absolute position of a given amino acid in accordance with the disclosure may vary from the corresponding position due to deletion or addition of amino acids elsewhere in a (mutant or wild-type) lipocalin. Similarly, the absolute position of a given nucleotide in accordance with the present disclosure may vary from the corresponding position due to deletions or additional nucleotides elsewhere in a mutein or wild-type lipocalin 5′-untranslated region (UTR) including the promoter and/or any other regulatory sequences or gene regions (including exons and introns).

Thus, for a “corresponding position” in accordance with the disclosure, it is preferably to be understood that the absolute positions of nucleotides or amino acids may differ from adjacent nucleotides or amino acids but said adjacent nucleotides or amino acids which may have been exchanged, deleted, or added may be comprised by the same one or more “corresponding positions”.

In addition, for a corresponding position in a lipocalin mutein based on a reference sequence in accordance with the disclosure, it is preferably to be understood that the positions of nucleotides or amino acids of a lipocalin mutein can structurally correspond to the positions elsewhere in a reference lipocalin (wild-type lipocalin) or another lipocalin mutein, even if they may differ in the absolute position numbers, as appreciated by the skilled person in light of the highly conserved overall folding pattern among lipocalins.

As used interchangeably herein, the terms “conjugate,” “conjugation,” “fuse,” “fusion,” or “linked” refer to the joining together of two or more subunits, through all forms of covalent or non-covalent linkage, by means including, but not limited to, genetic fusion, chemical conjugation, coupling through a linker or a cross-linking agent, and non-covalent association.

The term “fusion polypeptide” or “fusion protein” as used interchangeably herein refers to a polypeptide or protein comprising two or more subunits. In some embodiments, a fusion polypeptide as described herein comprises two or more subunits, wherein at least one of these subunits binds to CTGF. In some embodiments, at least two of these subunits bind to CTGF. Within the fusion polypeptide, these subunits may be linked by covalent or non-covalent linkage. Preferably, the fusion polypeptide is a translational fusion between the two or more subunits. The translational fusion may be generated by genetically engineering the coding sequence for one subunit in a reading frame with the coding sequence of a further subunit. Both subunits may be interspersed by a nucleotide sequence encoding a linker. However, the subunits of a fusion polypeptide of the present disclosure may also be linked through chemical conjugation. The subunits forming the fusion polypeptide are typically linked to each other as follows: C-terminus of one subunit to N-terminus of another subunit, or C-terminus of one subunit to C-terminus of another subunit, or N-terminus of one subunit to N-terminus of another subunit, or N-terminus of one subunit to C-terminus of another subunit. The subunits of the fusion polypeptide can be linked in any order and may include more than one of any of the constituent subunits. If one or more of the subunits is part of a protein (complex) that consists of more than one polypeptide chain, the term “fusion polypeptide” may also refer to the polypeptide comprising the fused sequences and all other polypeptide chain(s) of the protein (complex).

As used herein, the term “subunit” of a fusion protein/polypeptide disclosed herein refers to a single protein or a separate polypeptide chain, which can form a stable folded structure by itself and defines a unique function of providing a binding motif towards a target. In some embodiments, a preferred subunit of the disclosure is a lipocalin mutein.

A “linker” that may be comprised by a fusion protein or polypeptide of the present disclosure joins together two or more subunits of a fusion polypeptide as described herein. The linkage can be covalent or non-covalent. A preferred covalent linkage is via a peptide bond, such as a peptide bond between amino acids. A preferred linker is a peptide linker. Accordingly, in a preferred embodiment, said linker comprises one or more amino acids, such as 1, 2, 3, 4, 5, 6, 7, 8, 9, 10, 11, 12, 13, 14, 15, 16, 17, 18, 19, 20 or more amino acids. Preferred peptide linkers are described herein, including glycine-serine (GS) linkers, glycosylated GS linkers, and proline-alanine-serine polymer (PAS) linkers. In some preferred embodiments, a GS linker is a (G₄S)₃ as described in SEQ ID NO: 42 and is used to join together the subunits of a fusion polypeptide. Other preferred linkers include chemical linkers.

As used herein, the term “albumin” includes all mammal albumins such as human serum albumin or bovine serum albumin or rat serum albumin.

A “sample” is defined as a biological sample taken from any subject. Biological samples include, but are not limited to, blood, serum, urine, feces, semen, or tissue, including tumor tissue.

A “subject” is a vertebrate, preferably a mammal, more preferably a human. The term “mammal” is used herein to refer to any animal classified as a mammal, including, without limitation, humans, domestic and farm animals, and zoo, sports, or pet animals, such as sheep, dogs, horses, cats, cows, rats, pigs, apes such as cynomolgus monkeys, to name only a few illustrative examples. Preferably, the “mammal” used herein is human.

An “effective amount” is an amount sufficient to yield beneficial or desired results. An effective amount can be administered in one or more doses.

As used herein, “antibody” includes whole antibodies or any antigen binding fragment (i.e. “antigen-binding portion”) or single chain thereof. A whole antibody refers to a glycoprotein comprising at least two heavy chains (HCs) and two light chains (LCs) inter-connected by disulfide bonds. Each heavy chain is comprised of a heavy chain variable domain (V_{H} or HCVR) and a heavy chain constant region (C_{H}). The heavy chain constant region is comprised of three domains, C_{H1}, C_{H2} and C_{H3}. Each light chain is comprised of a light chain variable domain (V_{L} or LCVR) and a light chain constant region (C_{L}). The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} is composed of three CDRs and four FRs, arranged in the following order from the amino-terminus to the carboxy-terminus: FR1, CDR1, FR2, CDR2, FR3, CDR3, FR4. The variable regions of the heavy and light chains contain a binding domain that interacts with an antigen (e.g., CTGF). The constant regions of the antibodies may optionally mediate the binding of the immunoglobulin to host tissues or factors, including various cells of the immune system (e.g., effector cells) and the first component (C1q) of the classical complement system.

As used herein, “antigen binding fragment” of an antibody refers to one or more fragments of an antibody that retain the ability to specifically bind to an antigen (e.g., CTGF). It has been shown that the antigen-binding function of an antibody can be performed by fragments of a full-length antibody. Examples of binding fragments encompassed within the term “antigen-binding fragment” of an antibody include (i) a Fab fragment consisting of the V_{H}, V_{L}, C_{L} and C_{H1} domains; (ii) a F(ab′)₂ fragment comprising two Fab fragments linked by a disulfide bridge at the hinge region; (iii) a Fab′ fragment consisting of the V_{H}, V_{L}, C_{L} and C_{H1} domains and the region between C_{H1} and C_{H2} domains; (iv) an Fd fragment consisting of the V_{H} and C_{H1} domains; (v) a single-chain Fv fragment consisting of the V_{H} and V_{L} domains of a single arm of an antibody, (vi) a dAb fragment (Ward et al., Nature, 1989, 341, 544-546) consisting of a V_{H} domain; and (vii) an isolated complementarity determining region (CDR) or a combination of two or more isolated CDRs which may optionally be joined by a synthetic linker; (viii) a “diabody” comprising the V_{H} and V_{L} connected in the same polypeptide chain using a short linker (see, e.g., patent documents EP 404,097; WO 93/11161; and Holliger et al., Proc Natl Acad Sci USA, 1993, 90 (14) 6444-6448); (ix) a “domain antibody fragment” containing only the V_{H} or V_{L}, where in some instances two or more V_{H} regions are covalently joined.

Antibodies may be polyclonal or monoclonal; xenogeneic, allogeneic, or syngeneic; or modified forms thereof (e.g., humanized, chimeric, or multispecific). Antibodies may also be fully human.

As used herein, “framework” or “FR” refers to the variable domain residues other than the hypervariable region (CDR) residues.

“Fragment crystallizable region” or “Fc region” refers to the C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof (numbering according to EU index of Kabat (Johnson and Wu, Nucleic Acids Res, 2000, 28, 214-8). The C-terminal lysine (residue 447 according to EU index of Kabat) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the disclosure include human IgG1, IgG2 (IgG2A, IgG2B), IgG3, and IgG4.

“Fc receptor” or “FcR” refers to a receptor that binds to the Fc region of an antibody.

As used herein, “isolated antibody” refers to an antibody that is substantially free of its natural environment. For instance, an isolated antibody is substantially free of cellular material and other proteins from the cell or tissue source from which it is derived. An “isolated antibody” further refers to an antibody that is substantially free of other antibodies having different antigenic specificities. In an illustrative example, an isolated antibody that binds specifically CTGF is substantially free of antibodies that specifically bind antigens other than CTGF. However, an isolated antibody that specifically binds CTGF may have cross-reactivity with other antigens, such as CTGF molecules from other species.

As used herein, “monoclonal antibody” refers to a preparation of antibody molecules of single molecular composition. A monoclonal antibody composition displays a single binding specificity and affinity for a particular epitope.

As used herein, “humanized antibody” refers to an antibody that consists of the CDRs of antibodies derived from mammals other than human, and the FR region and the constant region of a human antibody. A humanized antibody is useful as an effective component in a therapeutic agent due to the reduced antigenicity.

As used herein, “human antibody” includes antibodies having variable regions in which both the framework and CDR regions are derived from human germline immunoglobulin sequences. Furthermore, if the antibody contains a constant region, the constant region is also derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

### III. DESCRIPTIONS OF FIGURES

FIG. 1: Illustrates the anti-fibrotic activity of a CTGF-targeting lipocalin mutein delivered via local administration to the lung in comparison to an anti-CTGF monoclonal antibody delivered intravenously at day 14 after bleomycin challenge in vivo. (A) shows Ashcroft scores as the median score per animal obtained from histopathological analyses of 10 individual tissue sections per subject. Graphs also indicate the median for each treatment group. The mean percent decline in Ashcroft score was calculated for each treatment group by normalization to the mean Ashcroft score of the respective vehicle control group. Statistical analysis was performed as described in the figure. (B) shows collagen1a1 (Col1a1) protein deposition as the % of Col1a1 positive lung surface area per animal determined by immunohistochemistry of lung tissue sections and subsequent quantitative analysis. The graph also indicates the median of all animals analyzed per treatment group. The treatment effect is indicated as the % decline of Col1a1 positive surface when compared to the mean of the respective vehicle control-treated animals of the same route of administration. Statistical analysis was performed as described in the figure.

FIG. 2: Illustrates the effect of CTGF-targeting lipocalin muteins on TGF-b1 impaired formation of organoids. CCL-206 lung fibroblasts were treated with TGF-b1 for 48 h and subsequently co-cultured with primary murine Epcam+ positive progenitor cells for 14 days. To analyze effects on organoid formation, co-cultured cells were treated with Nintedanib (100 nM) as a positive control, a lipocalin mutein scaffold control that does not bind to CTGF (100 nM), the fusion protein of SEQ ID NO: 74 (10 & 100 nM) and with an anti-CTGF monoclonal antibody (SEQ ID NOs: 60 and 61) over the whole time period. The figure represents the organoid formation as % normalized to the vehicle treated control. Single data points represent biological replicates generated with Epcam+ positive cells isolated from different mice (n=8, −/+SEM).

FIG. 3: Sequence alignment of hNGAL muteins.

FIG. 4: Shows binding of the exemplary lipocalin mutein of SEQ ID NO: 23 and of the exemplary fusion protein of SEQ ID NO: 74 to TGFβ-activated normal human lung fibroblasts (NHLFs), as measured by detection of the lipocalin scaffold by immunofluorescence staining. Signals were normalized to those of the respective controls (NGAL or NGAL-NGAL fusion).

FIG. 5: Shows the droplet size distribution of the CTGF-targeting lipocalin mutein of SEQ ID NO: 23 (A) and of the fusion protein of SEQ ID NO: 74 (B) upon nebulization with a vibrating mesh nebulizer in conjunction with the Malvern Spraytec and inhalation cell. In (A), 10% of the generated droplets are below 1.4 μm (Dv(10)), 50% are below 3.5 μm (Dv(50)) and 90% are below 8.6 μm (Dv(90)). In (B), 10% of the generated droplets are below 1.8 μm (Dv(10)), 50% are below 4.6 μm (Dv(50)) and 90% are below 10.5 μm (Dv(90)).

FIG. 6: Illustrates effective targeting of fibrotic lung tissue in mice with bleomycin-induced pulmonary fibrosis by CTGF-targeting lipocalin muteins and fusion proteins comprising them. (A) shows representative 3D overview images with the signals of the indicated fluorescently labeled compounds shown in glow scale (scale bars: 500 m). (B) shows magnified 2D sections from 3D scanned lungs with the signals of the indicated fluorescently labeled compounds shown in glow scale (scale bars: 150 m). (C) shows the total compound fluorescent signal of the indicated compounds in fibrotic areas of the lung (3D quantification of signal intensity). (D) shows the volume fraction of the fibrotic area targeted by the indicated compounds (3D quantification of the fibrotic area of the lung with compound-specific signal).

FIG. 7: Shows a comparison of the PK profiles of the exemplary lipocalin mutein of SEQ ID NO: 23 and of the anti-CTGF monoclonal antibody of SEQ ID NOs: 60 and 61. (A) shows PK analysis of the lipocalin mutein in bronchoalveolar lavage fluid (BALF), lung tissue and plasma. The lipocalin mutein (100 μg/mouse) was administered to the lungs of mice, and exposure in different compartments was measured after 2, 4, 8 and 24 h by ELISA. (B) shows the PK profile of the antibody in BALF, lung tissue and plasma. 100 μg antibody were administered to mice via intravenous infusion and exposure was measured after 1, 8, 24 and 96 h by ELISA.

### IV. DETAILED DESCRIPTION OF THE DISCLOSURE

In one aspect, the present disclosure provides human lipocalin muteins that bind CTGF and useful applications therefor. The disclosure also provides methods of making CTGF binding proteins described herein as well as compositions comprising such proteins. CTGF binding proteins of the disclosure as well as compositions thereof may be used in methods of detecting CTGF in a sample or in methods of binding of CTGF in a subject. No such human lipocalin muteins having these features attendant to the uses provided by present disclosure have been previously described.

### A. Lipocalin Muteins of the Disclosure.

Lipocalins are proteinaceous binding molecules that have naturally evolved to bind ligands. Lipocalins occur in many organisms, including vertebrates, insects, plants, and bacteria. The members of the lipocalin protein family (Pervaiz and Brew, 1987, FASEB J 1(3):209-14) are typically small, secreted proteins and have a single polypeptide chain. They are characterized by a range of different molecular-recognition properties: their binding to various, principally hydrophobic small molecules (such as retinoids, fatty acids, cholesterols, prostaglandins, biliverdins, pheromones, tastants, and odorants), and their binding to specific cell-surface receptors and their formation of macromolecular complexes. Although they have, in the past, been classified primarily as transport proteins, it is now clear that the lipocalins fulfill a variety of physiological functions. These include roles in retinol transport, olfaction, pheromone signaling, and the synthesis of prostaglandins. Lipocalins have also been implicated in the regulation of the immune response and the mediation of cell homeostasis (reviewed, e.g., in Flower et al., 2000 Biochim Biophys Acta, 1482, 9-24, Flower, 1996 Biochem J, 318 (Pt 1), 1-14).

Lipocalins share unusually low levels of overall sequence conservation, often with sequence identities of less than 20%. In strong contrast, their overall folding pattern is highly conserved. The central part of the lipocalin structure consists of a single eight-stranded anti-parallel β-sheet closed back on itself to form a continuously hydrogen-bonded β-barrel. This β-barrel forms a central cavity. One end of the barrel is sterically blocked by the N-terminal peptide segment that runs across its bottom as well as three peptide loops connecting the β-strands. The other end of the β-barrel is open to the solvent and encompasses a target-binding site, which is formed by four flexible peptide loops (AB, CD, EF, and GH). It is the diversity of the loops in the otherwise rigid lipocalin scaffold that gives rise to a variety of different binding modes each capable of accommodating targets of different size, shape, and chemical character (reviewed, e.g., in Skerra, 2000 Biochim Biophys Acta, 1482, 337-50, Flower et al., 2000, Biochim Biophys Acta, 1482, 9-24, Flower, 1996 Biochem J, 318 (Pt 1), 1-14).

A lipocalin mutein according to the present disclosure may be a mutein of any lipocalin. Examples of suitable lipocalins (also sometimes designated as “reference lipocalin,” “wild-type lipocalin,” “reference protein scaffolds,” or simply “scaffolds”) of which a mutein may be used include, but are not limited to, tear lipocalin (lipocalin-1, Tlc, or von Ebner's gland protein), retinol binding protein, neutrophil lipocalin-type prostaglandin D-synthase, β-lactoglobulin, bilin-binding protein (BBP), apolipoprotein D (APOD), neutrophil gelatinase-associated lipocalin (NGAL), α2-microglobulin-related protein (A2m), 24p3/uterocalin (24p3), von Ebner's gland protein 1 (VEGP 1), von Ebner's gland protein 2 (VEGP 2), and Major allergen Can f 1 (ALL-1). In particular embodiments, a lipocalin mutein is derived from the lipocalin group consisting of human tear lipocalin (hTlc), human neutrophil gelatinase-associated lipocalin (hNGAL), human apolipoprotein D (hAPOD) and the bilin-binding protein of Pieris brassicae.

The amino acid sequence of a lipocalin mutein according to the disclosure has a high sequence identity to the reference (or wild-type) lipocalin from which it is derived, preferably hNGAL, when compared to sequence identities with another lipocalin (see also above). In this general context the amino acid sequence of a lipocalin mutein according to the disclosure is at least substantially similar to the amino acid sequence of the corresponding reference (wild-type) lipocalin, with the proviso that there may be gaps (as defined herein) in an alignment that are the result of additions or deletions of amino acids. A respective sequence of a lipocalin mutein of the disclosure, being substantially similar to the sequences of the corresponding reference (wild-type) lipocalin, has, in some embodiments, at least 60%, at least 65%, at least 70%, at least 75%, at least 80%, at least 82%, at least 85%, at least 87%, or at least 90% identity, including at least 95% identity, to the sequence of the corresponding lipocalin. In this regard, a lipocalin mutein of the disclosure of course may contain, in comparison to the wild-type lipocalin, substitutions as described herein, which renders the lipocalin mutein capable of binding to CTGF.

Typically, a lipocalin mutein of the disclosure contains one or more mutated amino acid residues—relative to the amino acid sequence of the wild-type or reference lipocalin, for example, hNGAL—in the four loops at the open end that comprise a ligand-binding pocket and define the entrance of ligand-binding pocket (cf. above). As explained above, these regions are essential in determining the binding specificity of a lipocalin mutein for the desired target. A lipocalin mutein of the disclosure may also contain mutated amino acid residues in regions outside of the four loops. In some embodiments, a lipocalin mutein of the disclosure may contain one or more mutated amino acid residues in one or more of the three peptide loops (designated BC, DE, and FG) connecting the β-strands at the closed end of the lipocalin. In some particular embodiments, a mutein derived from a polypeptide of tear lipocalin, NGAL or a homologue thereof, may have 1, 2, 3, 4, or more mutated amino acid residues at any sequence position in the N-terminal region and/or in the three peptide loops BC, DE, and FG arranged at the end of the β-barrel structure that is located opposite to the natural lipocalin binding pocket. In some further embodiments, a mutein derived from tear lipocalin, NGAL or a homologue thereof, may have no mutated amino acid residues in peptide loop DE arranged at the end of the β-barrel structure, compared to the wild-type sequence of tear lipocalin, NGAL or a homologue thereof.

A lipocalin mutein according to the disclosure includes one or more, such as 2, 3, 4, 5, 6, 7, 8, 9, 10, 11, 12, 13, 14, 15, 16, 17, 18, 19, 20 or even more mutated amino acid residues in comparison to the amino acid sequence of the corresponding reference (wild-type) lipocalin, provided that such a lipocalin mutein should be capable of binding to CTGF. In some embodiments, a lipocalin mutein of the disclosure includes at least two, including 2, 3, 4, 5, or even more, mutated amino acid residues, where a native amino acid residue of the corresponding reference (wild-type) lipocalin is substituted by an arginine residue.

Any types and numbers of mutations, including substitutions, deletions, and insertions, are envisaged as long as the lipocalin mutein retains its capability to bind CTGF, and/or it has a sequence identity that it is at least 60%, such as at least 65%, at least 70%, at least 75%, at least 80%, at least 85% or higher identity to the amino acid sequence of the reference (wild-type) lipocalin, for example, mature hNGAL.

In some embodiments, a substitution is a conservative substitution. In some other embodiments, a substitution is a non-conservative substitution or one or more from the exemplary substitutions below.

Specifically, in order to determine whether the amino acid sequence of a lipocalin mutein is different from that of a reference (wild-type) lipocalin with regard to a certain position in the amino acid sequence of the reference (wild-type) lipocalin, a skilled artisan can use means and methods well-known in the art, e.g., alignments, either manually or by using computer programs such as BLAST2.0, which stands for Basic Local Alignment Search Tool or ClustalW or any other suitable program which is suitable to generate sequence alignments. Accordingly, the amino acid sequence of a reference (wild-type) lipocalin can serve as “subject sequence” or “reference sequence”, while the amino acid sequence of a lipocalin mutein serves as “query sequence” (see also above).

Conservative substitutions are generally the following substitutions, listed according to the amino acid to be mutated, each followed by one or more replacement(s) that can be taken to be conservative: Ala→Gly, Ser, or Val; Arg→Lys; Asn→Gln or His; Asp→Glu; Cys→Ser; Gln→Asn; Glu→Asp; Gly→Ala; His→Arg, Asn, or Gln; Ile→Leu or Val; Leu→Ile or Val; Lys→Arg, Gln, or Glu; Met→Leu, Tyr, or Ile; Phe→Met, Leu, or Tyr; Ser→Thr; Thr→Ser; Trp→Tyr; Tyr→Trp or Phe; Val→Ile or Leu. Other substitutions are also permissible and can be determined empirically or in accord with other known conservative or non-conservative substitutions. As a further orientation, the following eight groups each contain amino acids that can typically be taken to define conservative substitutions for one another:
a. Alanine (Ala), Glycine (Gly);
b. Aspartic acid (Asp), Glutamic acid (Glu);
c. Asparagine (Asn), Glutamine (Gln);
d. Arginine (Arg), Lysine (Lys);
e. Isoleucine (Ile), Leucine (Leu), Methionine (Met), Valine (Val);
f. Phenylalanine (Phe), Tyrosine (Tyr), Tryptophan (Trp);
g. Serine (Ser), Threonine (Thr); and
h. Cysteine (Cys), Methionine (Met).

If such conservative substitutions result in a change in biological activity, then more substantial changes, such as the following, or as further described below in reference to amino acid classes, may be introduced and the products be screened for a desired characteristic. Examples of such more substantial changes are: Ala→Leu or Ile; Arg→Gln; Asn→Asp, Lys, Arg, or His; Asp→Asn; Cys→Ala; Gln→Glu; Glu→Gln; His→Lys; Ile→Met, Ala, or Phe; Leu→Ala or Met; Lys→Asn; Met→Phe; Phe→Val, Ile, or Ala; Trp→Phe; Tyr→Thr or Ser; Val→Met, Phe, or Ala.

In some embodiments, substantial modifications in the physical and biological properties of the lipocalin (mutein) are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Naturally occurring residues are divided into groups based on common side-chain properties: (1) hydrophobic: methionine, alanine, valine, leucine, iso-leucine; (2) neutral hydrophilic: cysteine, serine, threonine; (3) acidic: aspartic acid, glutamic acid; (4) basic: histidine, lysine, arginine; (5) residues that influence chain orientation: glycine, proline; and (6) aromatic: tryptophan, tyrosine, phenylalanine. Non-conservative substitutions will entail exchanging a member of one of these classes for a member of another class.

Any cysteine residue not involved in maintaining the proper conformation of the respective lipocalin also may be substituted, generally with serine, to improve the oxidative stability of the molecule and prevent aberrant crosslinking. Conversely, cysteine bond (s) may be added to the lipocalin to improve its stability.

### B. CTGF-Specific Lipocalin Muteins of the Disclosure.

As noted above, a lipocalin is a polypeptide defined by its supersecondary structure, namely cylindrical β-pleated sheet supersecondary structural region comprising eight β-strands connected pair-wise by four loops at one end to define thereby a binding pocket. The present disclosure is not limited to lipocalin muteins specifically disclosed herein. In this regard, the disclosure relates to a lipocalin mutein having a cylindrical β-pleated sheet supersecondary structural region comprising eight β-strands connected pair-wise by four loops at one end to define thereby a binding pocket, wherein at least one amino acid of each of at least three of said four loops has been mutated and wherein said lipocalin is effective to bind CTGF with detectable affinity.

In one particular embodiment, a lipocalin mutein disclosed herein is a mutein of mature human neutrophil gelatinase-associated lipocalin (hNGAL). A mutein of mature hNGAL may be designated herein as an “hNGAL mutein”.

In one aspect, the present disclosure includes any number of lipocalin muteins derived from a reference (wild-type) lipocalin, preferably derived from mature hNGAL, that bind CTGF with detectable affinity. In a related aspect, the disclosure includes various lipocalin muteins that are capable of regulating the downstream signaling pathways of CTGF by binding to CTGF. In this sense, CTGF can be regarded as a non-natural target of the reference (wild-type) lipocalin, preferably hNGAL, where “non-natural target” refers to a substance that does not bind to the reference (wild-type) lipocalins under physiological conditions. By engineering reference (wild-type) lipocalins with one or more mutations at certain sequence positions, the present inventors have demonstrated that high affinity and high specificity for the non-natural target, CTGF, is possible. In some embodiments, at 1, 2, 3, 4, 5, 6, 7, 8, 9, 10, 11, 12 or even more nucleotide triplet(s) encoding certain sequence positions on wild-type lipocalins, a random mutagenesis may be carried out through substitution at these positions by a subset of nucleotide triplets, with the aim of generating a lipocalin mutein which is capable of binding CTGF.

The lipocalin muteins of the disclosure may have mutated, including substituted, deleted and inserted, amino acid residue(s) at one or more sequence positions corresponding to the linear polypeptide sequence of the reference lipocalin, preferably hNGAL. Preferably, the number of amino acid residues of a lipocalin mutein of the disclosure that is mutated in comparison with the amino acid sequence of the reference lipocalin, preferably hNGAL, is 1, 2, 3, 4, 5, 6, 7, 8, 9, 10, 11, 12, 13, 14, 15, 16, 17, 18, 19, 20 or more such as 25, 30, 35, 40, 45 or 50, with 1, 2, 3, 4, 5, 6, 7, 8, 9, 10, or 11 being preferred and 9, 10 or 11 being even more preferred. However, it is preferred that a lipocalin mutein of the disclosure is still capable of binding CTGF.

In some embodiments, the present disclosure encompasses hNGAL muteins as defined above, in which one or more amino acid residues, such as Ile at position 41 of the linear polypeptide sequence of the mature human lipocalin 2 (hNGAL) (SEQ ID NO: 1) has been deleted. Further, a lipocalin mutein of the disclosure may include the wild-type (natural) amino acid sequence of the reference (wild-type) lipocalin, preferably hNGAL, outside the mutated amino acid sequence positions.

In some preferred embodiments, the one or more mutated amino acid residues carried by a lipocalin mutein of the disclosure does, at least essentially, not hamper or not interfere with the binding activity to the designated target and the folding of the mutein. Such mutations, including substitution, deletion and insertion, can be accomplished at the DNA level using established standard methods (Sambrook and Russell, 2001, Molecular cloning: a laboratory manual, Cold Spring Harbor, N.Y., Cold Spring Harbor Laboratory Press). In some embodiments, the mutated amino acid residue(s) at one or more sequence positions corresponding to the linear polypeptide sequence of the reference (wild-type) lipocalin, preferably hNGAL, is introduced through random mutagenesis by substituting the nucleotide triplet(s) encoding the corresponding sequence positions of the reference lipocalin with a subset of nucleotide triplets.

In some embodiments, a lipocalin mutein that binds CTGF with detectable affinity may include at least one amino acid substitution of a native cysteine residue by another amino acid, for example, a serine residue. In some other embodiments, a lipocalin mutein that binds CTGF with detectable affinity may include one or more non-native cysteine residues substituting one or more amino acids of a reference (wild-type) lipocalin, preferably hNGAL. In a further particular embodiment, a lipocalin mutein according to the disclosure includes at least two amino acid substitutions of a native amino acid by a cysteine residue, hereby to form one or more cysteine bridges. In some embodiments, said cysteine bridge may connect at least two loop regions. The definition of these regions is used herein in accordance with Flower (1996) Biochem J, 318 (Pt 1), 1-14, Flower (2000) Biochim Biophys Acta, 1482, 327-36 and Breustedt et al. (2005) J Biol Chem, 280, 484-93.

Generally, a lipocalin mutein of the disclosure may have about at least 70%, including at least about 80%, such as at least about 85% amino acid sequence identity, with the amino acid sequence of the mature hNGAL (SEQ ID NO: 1).

In some embodiments, the present disclosure provides CTGF-binding lipocalin muteins. In this regard, the disclosure provides one or more lipocalin muteins that are capable of binding (human) CTGF with a detectable affinity, preferably with an affinity measured by a K_{D} of about 10⁻⁵ M or lower. The preferred lipocalin muteins are capable of binding CTGF with an affinity measured by a K_{D} of about 500 nM or lower, about 400 nM or lower, about 300 nM or lower, about 200 nM or lower, or about 100 nM or lower. Some preferred lipocalin muteins are even capable of binding CTGF with an affinity measured by a K_{D} of about 90 nM or lower, about 80 nM or lower, about 70 nM or lower, about 60 nM or lower, about 50 nM or lower, about 40 nM or lower, about 30 nM or lower, about 20 nM or lower, about 10 nM or lower. Even more preferred lipocalin muteins are even capable of binding CTGF with an affinity measured by a K_{D} of about 9 nM or lower, about 8 nM or lower, about 7 nM or lower, about 6 nM or lower, about 5 nM or lower, about 4 nM or lower, about 3 nM or lower, about 2 nM or lower, about 1 nM or lower, about 0.9 nM or lower, about 0.8 nM or lower, about 0.7 nM or lower, about 0.6 nM or lower, about 0.5 nM or lower, about 0.4 nM or lower, about 0.3 nM or lower, about 0.2 nM or lower, about 0.1 nM or lower, about 0.09 nM or lower, about 0.08 nM or lower, about 0.07 nM or lower, or even about 0.06 nM or lower. In some embodiments, the lipocalin muteins are capable of binding CTGF with an affinity measured by a K_{D} which is lower than that of the anti-CTGF monoclonal antibody of SEQ ID NOs: 60 and 61. Some CTGF-binding lipocalin muteins of the disclosure may be cross-reactive with cynomolgus CTGF (cyCTGF). Such lipocalin muteins may bind to cynomolgus CTGF with an affinity measured by a K_{D} of about 200 nM or lower, about 100 nM or lower, about 90 nM or lower, about 80 nM or lower, about 70 nM or lower, about 60 nM or lower, about 50 nM or lower, about 40 nM or lower, about 30 nM or lower, about 20 nM or lower, about 10 nM or lower, about 9 nM or lower, about 8 nM or lower, about 7 nM or lower, about 6 nM or lower, about 5 nM or lower, about 4 nM or lower, about 3 nM or lower, about 2 nM or lower, about 1 nM or lower, or about 0.5 nM or lower. Some CTGF-binding lipocalin muteins of the disclosure may be cross-reactive with murine CTGF (mCTGF). Such lipocalin muteins may bind to murine CTGF with an affinity measured by a K_{D} of about 200 nM or lower, about 100 nM or lower, about 90 nM or lower, about 80 nM or lower, about 70 nM or lower, about 60 nM or lower, about 50 nM or lower, about 40 nM or lower, about 30 nM or lower, about 20 nM or lower, about 10 nM or lower, about 9 nM or lower, about 8 nM or lower, about 7 nM or lower, about 6 nM or lower, about 5 nM or lower, about 4 nM or lower, about 3 nM or lower, about 2 nM or lower, about 1 nM or lower, or about 0.5 nM or lower. Some CTGF-binding lipocalin muteins of the disclosure may be cross-reactive with rat CTGF (rCTGF). Such lipocalin muteins may bind to rat CTGF with an affinity measured by a K_{D} of about 200 nM or lower, about 100 nM or lower, about 90 nM or lower, about 80 nM or lower, about 70 nM or lower, about 60 nM or lower, about 50 nM or lower, about 40 nM or lower, about 30 nM or lower, about 20 nM or lower, about 10 nM or lower, about 9 nM or lower, about 8 nM or lower, about 7 nM or lower, about 6 nM or lower, about 5 nM or lower, about 4 nM or lower, about 3 nM or lower, about 2 nM or lower, about 1 nM or lower, or about 0.5 nM or lower. Such affinities can be determined, for example, by SPR analysis, such as essentially described in Example 5.

A lipocalin mutein or a fusion protein of the disclosure may be capable of binding CTGF with an EC50 value of about 200 nM or lower, about 100 nM or lower, about 90 nM or lower, about 80 nM or lower, about 70 nM or lower, about 60 nM or lower, about 50 nM or lower, about 40 nM or lower, about 30 nM or lower, about 20 nM or lower, about 10 nM or lower, about 9 nM or lower, about 8 nM or lower, about 7 nM or lower, about 6 nM or lower, about 5 nM or lower, about 4 nM or lower, about 3 nM or lower, about 2 nM or lower, about 1 nM or lower, or about 0.5 nM or lower. The EC50 value can be determined, for example, by ELISA, such as essentially described in Example 6. Alternatively, the EC50 value can be determined, for example, by Homogeneous Time Resolved Fluorescence assay, such as essentially described in Example 7.

Some CTGF-binding lipocalin muteins of the disclosure are capable of competing or competes with an antibody having the heavy chain CDR sequences GFTFSSYG (CDR1, SEQ ID NO: 53), IGTGGGT (CDR2, SEQ ID NO: 54), and ARGDYYGSGSFFDC (CDR3, SEQ ID NO: 55), and the light chain CDR sequences QGISSW (CDR1, SEQ ID NO: 56), AAS (CDR2), and QQYNSYPPT (CDR3, SEQ ID NO: 57); the V_{H} and V_{L} sequences of SEQ ID NOs: 58 and 59; and/or the heavy chain and light chain sequences of SEQ ID NOs: 60 and 61 for the binding of CTGF. Some other CTGF-binding lipocalin muteins of the disclosure do not compete with an antibody having the heavy chain CDR sequences GFTFSSYG (CDR1, SEQ ID NO: 53), IGTGGGT (CDR2, SEQ ID NO: 54), and ARGDYYGSGSFFDC (CDR3, SEQ ID NO: 55), and the light chain CDR sequences QGISSW (CDR1, SEQ ID NO: 56), AAS (CDR2), and QQYNSYPPT (CDR3, SEQ ID NO: 57); the V_{H} and V_{L} sequences of SEQ ID NOs: 58 and 59; and/or the heavy chain and light chain sequences of SEQ ID NOs: 60 and 61 for the binding of CTGF. Competition for the binding of CTGF can be determined, for example, by SPR analysis, such as essentially described in Example 8.

Some CTGF-binding lipocalin muteins of the disclosure bind to an epitope on CTGF that does not overlap with the target epitope of an antibody having the heavy chain CDR sequences GFTFSSYG (CDR1, SEQ ID NO: 53), IGTGGGT (CDR2, SEQ ID NO: 54), and ARGDYYGSGSFFDC (CDR3, SEQ ID NO: 55), and the light chain CDR sequences QGISSW (CDR1, SEQ ID NO: 56), AAS (CDR2), and QQYNSYPPT (CDR3, SEQ ID NO: 57); the V_{H} and V_{L} sequences of SEQ ID NOs: 58 and 59; and/or the heavy chain and light chain sequences of SEQ ID NOs: 60 and 61 for the binding of CTGF. Some other CTGF-binding lipocalin muteins of the disclosure bind to an epitope on CTGF that overlaps with the target epitope of an antibody having the heavy chain CDR sequences GFTFSSYG (CDR1, SEQ ID NO: 53), IGTGGGT (CDR2, SEQ ID NO: 54), and ARGDYYGSGSFFDC (CDR3, SEQ ID NO: 55), and the light chain CDR sequences QGISSW (CDR1, SEQ ID NO: 56), AAS (CDR2), and QQYNSYPPT (CDR3, SEQ ID NO: 57); the V_{H} and V_{L} sequences of SEQ ID NOs: 58 and 59; and/or the heavy chain and light chain sequences of SEQ ID NOs: 60 and 61 for the binding of CTGF.

Some CTGF-binding lipocalin muteins of the disclosure are capable of binding to a fragment of CTGF that comprises domains 1 and 2 but lacks domains 3 and 4. Some other CTGF-binding lipocalin muteins of the disclosure do not bind to a fragment of CTGF that comprises domains 1 and 2 but lacks domains 3 and 4, but are capable of binding to full-length CTGF. Binding to different domains can be determined, for example, by an ELISA assay, such as an assay as essentially described in Example 9.

A CTGF-binding lipocalin mutein of the disclosure are preferably not cross-reactive with other members of the CCN protein family. Accordingly, a CTGF-binding lipocalin of the disclosure is not cross-reactive with one or more members of the CCN protein family selected from the group consisting of (human) CYR61 (CCN1), (human) NOV (CCN3), (human) WISP-1 (CCN4), (human) WISP-2 (CCN5), and (human) WISP-3 (CCN6). A CTGF-binding lipocalin of the disclosure is preferably not cross-reactive with all the afore-mentioned other members of the CCN protein family. Cross-reactivity with other members of the CCN protein family can be determined, for example, by an ELISA assay, such as an assay as essentially described in Example 10.

A CTGF-binding lipocalin mutein of the disclosure may provide an anti-fibrotic effect in vivo. Such anti-fibrotic effect may be expressed by an Ashcroft score. Some CTGF-binding lipocalin muteins are capable of providing a decrease of the Ashcroft score that is as low or lower as compared to the reference antibody of SEQ ID NOs: 60 and 61. Additionally or alternatively, such anti-fibrotic effect may be expressed by Collagen1a1 (Col1a1) deposition in the lung. Some CTGF-binding lipocalin muteins are capable of providing a degree of Col1a1 deposition that is as low or lower as compared to the reference antibody of SEQ ID NOs: 60 and 61. The reference antibody is preferably systemically administered, while the lipocalin mutein is preferably locally administered to the lung. Such anti-fibrotic effect can, for example, be measured in a bleomycin mouse model of lung fibrosis, such as essentially described in Example 11.

A CTGF-binding lipocalin mutein of the disclosure can be classified by their binding characteristics. An hNGAL mutein that does not compete with the antibody shown in SEQ ID NOs: 60 and 61 for CTGF binding, and that is capable of binding a fragment comprising only domains 1 and 2 of CTGF is considered to belong to the “N group”. Without wishing to be bound by theory it is believed that an hNGAL mutein of the N group binds to domain 1 and/or 2 of CTGF. An hNGAL mutein that competes with the antibody shown in SEQ ID NOs: 60 and 61 for CTGF binding, and that is capable of binding a fragment comprising only domains 1 and 2 of CTGF is considered to belong to the “NP group”. Without wishing to be bound by theory it is believed that an hNGAL mutein of the NP group binds to domain 2 of CTGF. An hNGAL mutein that does not compete with the antibody shown in SEQ ID NOs: 60 and 61 for CTGF binding, and that does not bind a fragment comprising only domains 1 and 2 of CTGF but is capable to bind full-length CTGF is considered to belong to the “C group”. Without wishing to be bound by theory it is believed that an hNGAL mutein of the C group binds to domain 3 and/or 4 of CTGF.

In one aspect, the present disclosure provides CTGF-binding hNGAL muteins.

In some embodiments, such hNGAL mutein may contain a mutated amino acid residue at one or more positions corresponding to positions 28, 36, 40, 41, 44, 47, 49, 52, 65, 68, 70, 72, 73, 74, 75, 77, 79, 80, 81, 87, 94, 95, 96, 97, 98, 99, 100, 102, 103, 104, 106, 110, 123, 125, 127, 128, 129, 130, 132, 134, and 136 of the linear polypeptide sequence of mature hNGAL (SEQ ID NO: 1).

In some embodiments, the hNGAL mutein may comprise at least 1, 2, 3, 4, 5, 6, 7, 8, 9, 10, 11, 12, 13, 14, 15, 16, 17, 18, 19, 20, 21, 22, 23, 24, or even more, mutated amino acid residues at one or more sequence positions corresponding to sequence positions 28, 36, 40, 41, 44, 47, 49, 52, 65, 68, 70, 72, 73, 74, 75, 77, 79, 80, 81, 87, 94, 95, 96, 97, 98, 99, 100, 102, 103, 104, 106, 110, 123, 125, 127, 128, 129, 130, 132, 134, and 136 of the linear polypeptide sequence of mature hNGAL (SEQ ID NO: 1), and wherein said polypeptide binds CTGF, in particular human CTGF. In some embodiments, an hNGAL mutein of the disclosure comprises 10 or more mutated amino acid residues at one or more of the above-mentioned positions of the linear polypeptide sequence of mature hNGAL (SEQ ID NO: 1). In some embodiments, an hNGAL mutein of the disclosure comprises 15 or more mutated amino acid residues at one or more of the above-mentioned positions of the linear polypeptide sequence of mature hNGAL (SEQ ID NO: 1). In some embodiments, an hNGAL mutein of the disclosure comprises 20 or more mutated amino acid residues at one or more of the above-mentioned positions of the linear polypeptide sequence of mature hNGAL (SEQ ID NO: 1).

In some embodiments, such hNGAL mutein may include a mutated amino acid residue at one or more positions corresponding to positions 28, 36, 40, 41, 47, 49, 52, 65, 68, 70, 72, 73, 77, 79, 81, 87, 94, 96, 100, 103, 106, 110, 125, 127, 132, and 134 of the linear polypeptide sequence of mature hNGAL (SEQ ID NO: 1).

In some embodiments, the hNGAL mutein may comprise at least 1, 2, 3, 4, 5, 6, 7, 8, 9, 10, 11, 12, 13, 14, 15, 16, 17, 18, 19, 20, 21, 22, 23, 24, or even more, mutated amino acid residues at one or more sequence positions corresponding to sequence positions 28, 36, 40, 41, 47, 49, 52, 65, 68, 70, 72, 73, 77, 79, 81, 87, 94, 96, 100, 103, 106, 110, 125, 127, 132, and 134 of the linear polypeptide sequence of mature hNGAL (SEQ ID NO: 1), and wherein said polypeptide binds CTGF, in particular human CTGF. In some embodiments, an hNGAL mutein of the disclosure comprises 10 or more mutated amino acid residues at one or more of the above-mentioned positions of the linear polypeptide sequence of mature hNGAL (SEQ ID NO: 1). In some embodiments, an hNGAL mutein of the disclosure comprises 15 or more mutated amino acid residues at one or more of the above-mentioned positions of the linear polypeptide sequence of mature hNGAL (SEQ ID NO: 1). In some embodiments, an hNGAL mutein of the disclosure comprises 20 or more mutated amino acid residues at one or more of the above-mentioned positions of the linear polypeptide sequence of mature hNGAL (SEQ ID NO: 1).

In some embodiments, such hNGAL mutein may include a mutated amino acid residue at one or more positions corresponding to positions 28, 36, 40, 41, 44, 47, 49, 52, 65, 70, 72, 73, 74, 75, 77, 79, 80, 81, 87, 94, 96, 98, 99, 100, 103, 104, 106, 123, 125, 127, 128, 129, 132, 134, and 136 of the linear polypeptide sequence of mature hNGAL (SEQ ID NO: 1).

In some embodiments, the hNGAL mutein may comprise at least 1, 2, 3, 4, 5, 6, 7, 8, 9, 10, 11, 12, 13, 14, 15, 16, 17, 18, 19, 20, 21, 22, 23, 24, or even more, mutated amino acid residues at one or more sequence positions corresponding to sequence positions 28, 36, 40, 41, 44, 47, 49, 52, 65, 70, 72, 73, 74, 75, 77, 79, 80, 81, 87, 94, 96, 98, 99, 100, 103, 104, 106, 123, 125, 127, 128, 129, 132, 134, and 136 of the linear polypeptide sequence of mature hNGAL (SEQ ID NO: 1), and wherein said polypeptide binds CTGF, in particular human CTGF. In some embodiments, an hNGAL mutein of the disclosure comprises 10 or more mutated amino acid residues at one or more of the above-mentioned positions of the linear polypeptide sequence of mature hNGAL (SEQ ID NO: 1). In some embodiments, an hNGAL mutein of the disclosure comprises 15 or more mutated amino acid residues at one or more of the above-mentioned positions of the linear polypeptide sequence of mature hNGAL (SEQ ID NO: 1). In some embodiments, an hNGAL mutein of the disclosure comprises 20 or more mutated amino acid residues at one or more of the above-mentioned positions of the linear polypeptide sequence of mature hNGAL (SEQ ID NO: 1).

In some embodiments, such hNGAL mutein may include a mutated amino acid residue at one or more positions corresponding to positions 28, 40, 41, 44, 47, 49, 52, 65, 70, 72, 73, 74, 75, 77, 79, 87, 94, 96, 98, 99, 100, 103, 104, 106, 125, 127, and 134 of the linear polypeptide sequence of mature hNGAL (SEQ ID NO: 1).

In some embodiments, the hNGAL mutein may comprise at least 1, 2, 3, 4, 5, 6, 7, 8, 9, 10, 11, 12, 13, 14, 15, 16, 17, 18, 19, 20, 21, 22, 23, 24, or even more, mutated amino acid residues at one or more sequence positions corresponding to sequence positions 28, 40, 41, 44, 47, 49, 52, 65, 70, 72, 73, 74, 75, 77, 79, 87, 94, 96, 98, 99, 100, 103, 104, 106, 125, 127, and 134 of the linear polypeptide sequence of mature hNGAL (SEQ ID NO: 1), and wherein said polypeptide binds CTGF, in particular human CTGF. In some embodiments, an hNGAL mutein of the disclosure comprises 10 or more mutated amino acid residues at one or more of the above-mentioned positions of the linear polypeptide sequence of mature hNGAL (SEQ ID NO: 1). In some embodiments, an hNGAL mutein of the disclosure comprises 15 or more mutated amino acid residues at one or more of the above-mentioned positions of the linear polypeptide sequence of mature hNGAL (SEQ ID NO: 1). In some embodiments, an hNGAL mutein of the disclosure comprises 20 or more mutated amino acid residues at one or more of the above-mentioned positions of the linear polypeptide sequence of mature hNGAL (SEQ ID NO: 1).

In some embodiments, such hNGAL mutein may include a mutated amino acid residue at one or more positions corresponding to positions 28, 40, 41, 44, 47, 49, 52, 65, 70, 72, 73, 74, 75, 77, 79, 87, 94, 96, 98, 99, 100, 103, 104, 106, 125, 127, and 134 of the linear polypeptide sequence of mature hNGAL (SEQ ID NO: 1).

In some embodiments, the hNGAL mutein may comprise at least 1, 2, 3, 4, 5, 6, 7, 8, 9, 10, 11, 12, 13, 14, 15, 16, 17, 18, 19, 20, 21, 22, 23, 24, or even more, mutated amino acid residues at one or more sequence positions corresponding to sequence positions 28, 40, 41, 44, 47, 49, 52, 65, 70, 72, 73, 74, 75, 77, 79, 87, 94, 96, 98, 99, 100, 103, 104, 106, 125, 127, and 134 of the linear polypeptide sequence of mature hNGAL (SEQ ID NO: 1), and wherein said polypeptide binds CTGF, in particular human CTGF. In some embodiments, an hNGAL mutein of the disclosure comprises 10 or more mutated amino acid residues at one or more of the above-mentioned positions of the linear polypeptide sequence of mature hNGAL (SEQ ID NO: 1). In some embodiments, an hNGAL mutein of the disclosure comprises 15 or more mutated amino acid residues at one or more of the above-mentioned positions of the linear polypeptide sequence of mature hNGAL (SEQ ID NO: 1). In some embodiments, an hNGAL mutein of the disclosure comprises 20 or more mutated amino acid residues at one or more of the above-mentioned positions of the linear polypeptide sequence of mature hNGAL (SEQ ID NO: 1).

In some embodiments, such hNGAL mutein may include a mutated amino acid residue at one or more positions corresponding to positions 28, 36, 40, 41, 47, 49, 52, 65, 68, 70, 72, 73, 77, 79, 81, 87, 94, 95, 96, 97, 98, 99, 100, 102, 103, 104, 106, 123, 127, 128, 129, 130, 132, 134, and 136 of the linear polypeptide sequence of mature hNGAL (SEQ ID NO: 1).

In some embodiments, the hNGAL mutein may comprise at least 1, 2, 3, 4, 5, 6, 7, 8, 9, 10, 11, 12, 13, 14, 15, 16, 17, 18, 19, 20, 21, 22, 23, 24, or even more, mutated amino acid residues at one or more sequence positions corresponding to sequence positions 28, 36, 40, 41, 47, 49, 52, 65, 68, 70, 72, 73, 77, 79, 81, 87, 94, 95, 96, 97, 98, 99, 100, 102, 103, 104, 106, 123, 127, 128, 129, 130, 132, 134, and 136 of the linear polypeptide sequence of mature hNGAL (SEQ ID NO: 1), and wherein said polypeptide binds CTGF, in particular human CTGF. In some embodiments, an hNGAL mutein of the disclosure comprises 10 or more mutated amino acid residues at one or more of the above-mentioned positions of the linear polypeptide sequence of mature hNGAL (SEQ ID NO: 1). In some embodiments, an hNGAL mutein of the disclosure comprises 15 or more mutated amino acid residues at one or more of the above-mentioned positions of the linear polypeptide sequence of mature hNGAL (SEQ ID NO: 1). In some embodiments, an hNGAL mutein of the disclosure comprises 20 or more mutated amino acid residues at one or more of the above-mentioned positions of the linear polypeptide sequence of mature hNGAL (SEQ ID NO: 1).

In some embodiments, such hNGAL mutein may include a mutated amino acid residue at one or more positions corresponding to positions 28, 36, 40, 41, 49, 52, 65, 68, 70, 72, 73, 77, 79, 81, 87, 96, 99, 100, 102, 103, 104, 106, 123, 127, 128, 129, 130, 132, 134, and 136 of the linear polypeptide sequence of mature hNGAL (SEQ ID NO: 1).

In some embodiments, the hNGAL mutein may comprise at least 1, 2, 3, 4, 5, 6, 7, 8, 9, 10, 11, 12, 13, 14, 15, 16, 17, 18, 19, 20, 21, 22, 23, 24, or even more, mutated amino acid residues at one or more sequence positions corresponding to sequence positions 28, 36, 40, 41, 49, 52, 65, 68, 70, 72, 73, 77, 79, 81, 87, 96, 99, 100, 102, 103, 104, 106, 123, 127, 128, 129, 130, 132, 134, and 136 of the linear polypeptide sequence of mature hNGAL (SEQ ID NO: 1), and wherein said polypeptide binds CTGF, in particular human CTGF. In some embodiments, an hNGAL mutein of the disclosure comprises 10 or more mutated amino acid residues at one or more of the above-mentioned positions of the linear polypeptide sequence of mature hNGAL (SEQ ID NO: 1). In some embodiments, an hNGAL mutein of the disclosure comprises 15 or more mutated amino acid residues at one or more of the above-mentioned positions of the linear polypeptide sequence of mature hNGAL (SEQ ID NO: 1). In some embodiments, an hNGAL mutein of the disclosure comprises 20 or more mutated amino acid residues at one or more of the above-mentioned positions of the linear polypeptide sequence of mature hNGAL (SEQ ID NO: 1).

In some embodiments, such hNGAL mutein may include a mutated amino acid residue at one or more positions corresponding to positions 28, 36, 40, 41, 47, 49, 52, 65, 68, 70, 72, 73, 77, 79, 81, 87, 94, 95, 96, 97, 98, 99, 100, 102, 103, 104, 106, 127, 132, and 134 of the linear polypeptide sequence of mature hNGAL (SEQ ID NO: 1).

In some embodiments, the hNGAL mutein may comprise at least 1, 2, 3, 4, 5, 6, 7, 8, 9, 10, 11, 12, 13, 14, 15, 16, 17, 18, 19, 20, 21, 22, 23, 24, or even more, mutated amino acid residues at one or more sequence positions corresponding to sequence positions 28, 36, 40, 41, 47, 49, 52, 65, 68, 70, 72, 73, 77, 79, 81, 87, 94, 95, 96, 97, 98, 99, 100, 102, 103, 104, 106, 127, 132, and 134 of the linear polypeptide sequence of mature hNGAL (SEQ ID NO: 1), and wherein said polypeptide binds CTGF, in particular human CTGF. In some embodiments, an hNGAL mutein of the disclosure comprises 10 or more mutated amino acid residues at one or more of the above-mentioned positions of the linear polypeptide sequence of mature hNGAL (SEQ ID NO: 1). In some embodiments, an hNGAL mutein of the disclosure comprises 15 or more mutated amino acid residues at one or more of the above-mentioned positions of the linear polypeptide sequence of mature hNGAL (SEQ ID NO: 1). In some embodiments, an hNGAL mutein of the disclosure comprises 20 or more mutated amino acid residues at one or more of the above-mentioned positions of the linear polypeptide sequence of mature hNGAL (SEQ ID NO: 1).

In some embodiments, a lipocalin mutein according to the disclosure may include at least one amino acid substitution of a native cysteine residue by, e.g., a serine residue. In some embodiments, an hNGAL mutein according to the disclosure includes an amino acid substitution of a native cysteine residue at positions 76 and/or 175 by another amino acid such as a serine residue. In this context it is noted that it has been found that removal of the structural disulfide bond (on the level of a respective naïve nucleic acid library) of wild-type hNGAL that is formed by the cysteine residues 76 and 175 (cf. Breustedt et al., J Biol Chem, 2005, 280, 484-93) may provide hNGAL muteins that are not only stably folded but are also able to bind a given non-natural target with high affinity. In some embodiments, the elimination of the structural disulfide bond may provide the further advantage of allowing for the generation or deliberate introduction of non-natural disulfide bonds into muteins of the disclosure, thereby, increasing the stability of the muteins. However, hNGAL muteins that bind CTGF and that have the disulfide bridge formed between Cys 76 and Cys 175 are also part of the present disclosure. In some embodiments, an hNGAL mutein according to the disclosure may include a mutation at position Cys 87 of mature hNGAL. For example, the cysteine residue can be replaced by another amino acid residue, such as serine.

In some embodiments, the hNGAL mutein of the disclosure may comprise a mutation at position Gln 28 of the linear polypeptide sequence of mature hNGAL (SEQ ID NO: 1). Such a mutation may be a Gln 28→His mutation, which may introduce a BstXI restriction site and which may facilitate cloning.

In some embodiments, the hNGAL mutein of the disclosure may comprise a mutation at position Asn 65 of the linear polypeptide sequence of mature hNGAL (SEQ ID NO: 1). Such a mutation may be an Asn 65→Asp, Gln, or Glu mutation, preferably an Asn 65→Asp mutation.

In some embodiments, a CTGF-binding hNGAL mutein according to the disclosure includes, at one or more positions corresponding to positions 28, 36, 40, 41, 44, 47, 49, 52, 65, 70, 72, 73, 74, 75, 77, 79, 80, 81, 87, 94, 95, 96, 97, 98, 99, 100, 102, 103, 104, 106, 110, 123, 125, 127, 128, 129, 130, 132, 134, and 136 of the linear polypeptide sequence of mature hNGAL (SEQ ID NO: 1), one or more of the following mutated amino acid residues: Gln 28→His; Leu 36→Arg, Lys, Ile, Val, Met, or Trp; Ala 40→Asn, Tyr, Lys, Phe, Ile, or Val; Ile 41→Arg, deletion of Ile 41, Gln, Gly, or Lys; Glu 44→Thr, Ile, Asp, Val, or Pro; Asp 47→Glu, Ser, Arg, Gln, or Tyr; Gln 49→Pro, Ser, Ala, Phe, Leu, or Ala; Tyr 52→Trp, Phe, Gly, or Ser; Asn 65→Asp; Ser 68→His, Gln, or Glu; Leu 70→His, Arg, Gln, or Val; Arg 72→Met, Leu, Ser, Glu, or Asp; Lys 73→Thr, Gln, Ala, Asn, or Asp; Lys 74→Glu or Arg; Lys 75→Arg or Ser; Asp 77→Arg, Lys, His, Ser, Val, Ile, or Leu; Trp 79→Ile, Leu, Thr, or Val; Ile 80→Ser; Arg 81→Asp, Lys, or Glu; Cys 87→Ser; Leu 94→Ile, Ala, Thr, Ser, Arg, His, or Glu; Gly 95→Ser; Asn 96→Ala, Ser, Tyr, Gln, Asp, or Pro; Ile 97→Tyr; Lys 98→Gly or Ser; Ser 99→Asn, Val, or Arg; Tyr 100→Gly, Arg, Ala, His, Phe, Pro, or Ser; Gly 102→Thr or Arg; Leu 103→Met, Gln, Ser, Phe, Leu, Glu, or Tyr; Thr 104→Tyr, Glu, Val, or Trp; Tyr 106→Pro, Ser, Thr, Gln, His, or Asp; Val 110→Ile; Phe 123→Trp, His, Ala, Leu, or Val; Lys 125→Trp, Ser, His, or Ala; Ser 127→Asn, Thr, Ile, Ala, Gln, Arg, Tyr, Trp, Phe, His, or Gly; Gln 128→Gly, Leu, or Pro; Asn 129→Thr, Ala, or Ser; Arg 130→Glu or Leu; Tyr 132→Trp, Thr, Ser, Phe, Ile, His, or Val; Lys 134→Thr, Ala, Val, Asn, Phe, Trp, His, or Gln; and Thr 136→Ala or Val. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1).

In some embodiments, a CTGF-binding hNGAL mutein according to the disclosure includes, at one or more positions corresponding to positions 28, 36, 40, 41, 44, 47, 49, 52, 65, 68, 70, 72, 73, 74, 75, 77, 79, 80, 81, 87, 94, 95, 96, 97, 98, 99, 100, 102, 103, 104, 106, 110, 123, 125, 127, 128, 129, 130, 132, 134, and 136 of the linear polypeptide sequence of mature hNGAL (SEQ ID NO: 1), one or more of the following mutated amino acid residues: Gln 28→His; Leu 36→Arg, Lys, Ile, Val, Met, or Trp; Ala 40→Asn, Tyr, Lys, Phe, Ile, or Val; Ile 41→Arg, deletion of Ile 41, Gln, Gly, or Lys; Glu 44→Thr, Ile, Asp, Val, or Pro; Asp 47→Glu, Ser, Arg, Gln, or Tyr; Gln 49→Pro, Ser, Ala, Phe, Leu, or Ala; Tyr 52→Trp, Phe, Gly, or Ser; Asn 65→Asp; Ser 68→His, Gln, or Glu; Leu 70→His, Arg, Gln, or Val; Arg 72→Met, Leu, Ser, Glu, or Asp; Lys 73→Thr, Gln, Ala, Asn, or Asp; Lys 74→Glu or Arg; Lys 75→Arg or Ser; Asp 77→Arg, Lys, His, Ser, Val, Ile, or Leu; Trp 79→Ile, Leu, Thr, or Val; Ile 80→Ser; Arg 81→Asp, Lys, or Glu; Cys 87→Ser; Leu 94→Ile, Ala, Thr, Ser, Arg, His, or Glu; Gly 95→Ser; Asn 96→Ala, Ser, Tyr, Gln, Asp, or Pro; Ile 97→Tyr; Lys 98→Gly or Ser; Ser 99→Asn, Val, or Arg; Tyr 100→Gly, Arg, Ala, His, Phe, Pro, or Ser; Gly 102→Thr or Arg; Leu 103→Met, Gln, Ser, Phe, Glu, or Tyr; Thr 104→Tyr, Glu, Val, or Trp; Tyr 106→Pro, Ser, Thr, Gln, His, or Asp; Val 110→Ile; Phe 123→Trp, His, Ala, Leu, or Val; Lys 125→Trp, Ser, His, or Ala; Ser 127→Asn, Thr, Ile, Ala, Gln, Arg, Tyr, Trp, Phe, His, or Gly; Gln 128→Gly, Leu, or Pro; Asn 129→Thr, Ala, or Ser; Arg 130→Glu or Leu; Tyr 132→Trp, Thr, Ser, Phe, Ile, His, or Val; Lys 134→Thr, Ala, Val, Asn, Phe, Trp, His, or Gln; and Thr 136→Ala or Val. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1).

In some embodiments, a CTGF-binding hNGAL mutein according to the disclosure includes, at one or more positions corresponding to positions 36, 40, 41, 44, 47, 49, 52, 70, 72, 73, 74, 75, 77, 79, 80, 81, 94, 95, 96, 97, 98, 99, 100, 102, 103, 104, 106, 110, 123, 125, 127, 128, 129, 130, 132, 134, and 136 of the linear polypeptide sequence of mature hNGAL (SEQ ID NO: 1), one or more of the following mutated amino acid residues: Leu 36→Arg, Lys, Ile, Val, Met, or Trp; Ala 40→Asn, Tyr, Lys, Phe, Ile, or Val; Ile 41→Arg, deletion of Ile 41, Gln, Gly, or Lys; Glu 44→Thr, Ile, Asp, Val, or Pro; Asp 47→Glu, Ser, Arg, Gln, or Tyr; Gln 49→Pro, Ser, Ala, Phe, Leu, or Ala; Tyr 52→Trp, Phe, Gly, or Ser; Ser 68→His, Gln, or Glu; Leu 70→His, Arg, Gln, or Val; Arg 72→Met, Leu, Ser, Glu, or Asp; Lys 73→Thr, Gln, Ala, Asn, or Asp; Lys 74→Glu or Arg; Lys 75→Arg or Ser; Asp 77→Arg, Lys, His, Ser, Val, Ile, or Leu; Trp 79→Ile, Leu, Thr, or Val; Ile 80→Ser; Arg 81→Asp, Lys, or Glu; Leu 94→Ile, Ala, Thr, Ser, Arg, His, or Glu; Gly 95→Ser; Asn 96→Ala, Ser, Tyr, Gln, Asp, or Pro; Ile 97→Tyr; Lys 98→Gly or Ser; Ser 99→Asn, Val, or Arg; Tyr 100→Gly, Arg, Ala, His, Phe, Pro, or Ser; Gly 102→Thr or Arg; Leu 103→Met, Gln, Ser, Phe, Leu, Glu, or Tyr; Thr 104→Tyr, Glu, Val, or Trp; Tyr 106→Pro, Ser, Thr, Gln, His, or Asp; Val 110→Ile; Phe 123→Trp, His, Ala, Leu, or Val; Lys 125→Trp, Ser, His, or Ala; Ser 127→Asn, Thr, Ile, Ala, Gln, Arg, Tyr, Trp, Phe, His, or Gly; Gln 128→Gly, Leu, or Pro; Asn 129→Thr, Ala, or Ser; Arg 130→Glu or Leu; Tyr 132→Trp, Thr, Ser, Phe, Ile, His, or Val; Lys 134→Thr, Ala, Val, Asn, Phe, Trp, His, or Gln; and Thr 136→Ala or Val. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1).

In some embodiments, a CTGF-binding hNGAL mutein according to the disclosure includes, at one or more positions corresponding to positions 36, 40, 41, 44, 47, 49, 52, 68, 70, 72, 73, 74, 75, 77, 79, 80, 81, 94, 95, 96, 97, 98, 99, 100, 102, 103, 104, 106, 110, 123, 125, 127, 128, 129, 130, 132, 134, and 136 of the linear polypeptide sequence of mature hNGAL (SEQ ID NO: 1), one or more of the following mutated amino acid residues: Leu 36→Arg, Lys, Ile, Val, Met, or Trp; Ala 40→Asn, Tyr, Lys, Phe, Ile, or Val; Ile 41→Arg, deletion of Ile 41, Gln, Gly, or Lys; Glu 44→Thr, Ile, Asp, Val, or Pro; Asp 47→Glu, Ser, Arg, Gln, or Tyr; Gln 49→Pro, Ser, Ala, Phe, Leu, or Ala; Tyr 52→Trp, Phe, Gly, or Ser; Ser 68→His, Gln, or Glu; Leu 70→His, Arg, Gln, or Val; Arg 72→Met, Leu, Ser, Glu, or Asp; Lys 73→Thr, Gln, Ala, Asn, or Asp; Lys 74→Glu or Arg; Lys 75→Arg or Ser; Asp 77→Arg, Lys, His, Ser, Val, Ile, or Leu; Trp 79→Ile, Leu, Thr, or Val; Ile 80→Ser; Arg 81→Asp, Lys, or Glu; Leu 94→Ile, Ala, Thr, Ser, Arg, His, or Glu; Gly 95→Ser; Asn 96→Ala, Ser, Tyr, Gln, Asp, or Pro; Ile 97→Tyr; Lys 98→Gly or Ser; Ser 99→Asn, Val, or Arg; Tyr 100→Gly, Arg, Ala, His, Phe, Pro, or Ser; Gly 102→Thr or Arg; Leu 103→Met, Gln, Ser, Phe, Glu, or Tyr; Thr 104→Tyr, Glu, Val, or Trp; Tyr 106→Pro, Ser, Thr, Gln, His, or Asp; Val 110→Ile; Phe 123→Trp, His, Ala, Leu, or Val; Lys 125→Trp, Ser, His, or Ala; Ser 127→Asn, Thr, Ile, Ala, Gln, Arg, Tyr, Trp, Phe, His, or Gly; Gln 128→Gly, Leu, or Pro; Asn 129→Thr, Ala, or Ser; Arg 130→Glu or Leu; Tyr 132→Trp, Thr, Ser, Phe, Ile, His, or Val; Lys 134→Thr, Ala, Val, Asn, Phe, Trp, His, or Gln; and Thr 136→Ala or Val. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1).

In some embodiments, a CTGF-binding hNGAL mutein according to the disclosure includes, at one or more positions corresponding to positions 28, 36, 40, 41, 47, 49, 52, 65, 68, 70, 72, 73, 77, 79, 81, 87, 94, 96, 100, 103, 106, 110, 125, 127, 132, and 134 of the linear polypeptide sequence of mature hNGAL (SEQ ID NO: 1), one or more of the following mutated amino acid residues: Gln 28→His; Leu 36→Arg or Lys; Ala 40→Asn; Ile 41→Arg, deletion of Ile 41, or Gln; Asp 47→Glu or Ser; Gln 49→Pro; Tyr 52→Trp; Asn 65→Asp; Ser 68→His; Leu 70→His; Arg 72→Met, Leu, or Ser; Lys 73→Thr; Asp 77→Arg or Lys; Trp 79→Ile or Leu; Arg 81→Asp; Cys 87→Ser; Leu 94→Ile or Ala; Asn 96→Ala; Tyr 100→Gly; Leu 103→Met; Tyr 106→Pro; Val 110→Ile; Lys 125→Trp; Ser 127→Asn or Thr; Tyr 132→Trp; and Lys 134→Thr. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein preferably does not compete with the antibody shown in SEQ ID NOs: 60 and 61 for CTGF binding, as, e.g., determined in an SPR assay, such as essentially described in Example 8. Such an hNGAL mutein preferably does not bind to a fragment comprising only domains 1 and 2 of CTGF, while being capable of binding to full-length CTGF, as, e.g., determined in an ELISA assay, such as essentially described in Example 9.

In some embodiments, a CTGF-binding hNGAL mutein according to the disclosure includes, at one or more positions corresponding to positions 36, 40, 41, 47, 49, 52, 68, 70, 72, 73, 77, 79, 81, 94, 96, 100, 103, 106, 110, 125, 127, 132, and 134 of the linear polypeptide sequence of mature hNGAL (SEQ ID NO: 1), one or more of the following mutated amino acid residues: Leu 36→Arg or Lys; Ala 40→Asn; Ile 41→Arg, deletion of Ile 41, or Gln; Asp 47→Glu or Ser; Gln 49→Pro; Tyr 52→Trp; Ser 68→His; Leu 70→His; Arg 72→Met, Leu, or Ser; Lys 73→Thr; Asp 77→Arg or Lys; Trp 79→Ile or Leu; Arg 81→Asp; Leu 94→Ile or Ala; Asn 96→Ala; Tyr 100→Gly; Leu 103→Met; Tyr 106→Pro; Val 110→Ile; Lys 125→Trp; Ser 127→Asn or Thr; Tyr 132→Trp; and Lys 134→Thr. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein preferably does not compete with the antibody shown in SEQ ID NOs: 60 and 61 for CTGF binding, e.g., as determined in an SPR assay, such as essentially described in Example 8. Such an hNGAL mutein preferably does not bind to a fragment comprising only domains 1 and 2 of CTGF, while being capable of binding to full-length CTGF, e.g., as determined in an ELISA assay, such as essentially described in Example 9.

In some embodiments, a CTGF-binding hNGAL mutein according to the disclosure includes, at one or more positions corresponding to positions 28, 36, 40, 41, 44, 47, 49, 52, 65, 70, 72, 73, 74, 75, 77, 79, 80, 81, 87, 94, 96, 98, 99, 100, 103, 104, 106, 123, 125, 127, 128, 129, 132, 134, and 136 of the linear polypeptide sequence of mature hNGAL (SEQ ID NO: 1), one or more of the following mutated amino acid residues: Gln 28→His; Leu 36→Ile or Val; Ala 40→Tyr or Lys; Ile 41→Gly; Glu 44→Thr, Ile, Asp, Val, or Pro; Asp 47→Arg, Gln, or Tyr; Gln 49→Ser or Ala; Tyr 52→Phe; Asn 65→Asp; Leu 70→Arg; Arg 72→Glu; Lys 73→Gln, Ala, or Asn; Lys 74→Glu or Arg; Lys 75→Arg or Ser; Asp 77→His, Lys, Ser, Val, or Ile; Trp 79→Thr; Ile 80→Ser; Arg 81→Lys; Cys 87→Ser; Leu 94→Ala, Thr, Ser, Arg, or His; Asn 96→Ser, Tyr, or Gln; Lys 98→Gly; Ser 99→Asn; Tyr 100→Arg, Ala or His; Leu 103→Gln or Ser; Thr 104→Tyr; Tyr 106→Ser or Thr; Phe 123→Trp, His, or Ala; Lys 125→Ser, His, or Ala; Ser 127→Ile, Thr, Ala, Gln, or Arg; Gln 128→Gly or Leu; Asn 129→Thr or Ala; Tyr 132→Thr, Ser, Phe, Ile, or His; Lys 134→Ala, Val, Asn, or Phe; and Thr 136→Ala. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein preferably competes with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, a CTGF-binding hNGAL mutein according to the disclosure includes, at one or more positions corresponding to positions 36, 40, 41, 44, 47, 49, 52, 70, 72, 73, 74, 75, 77, 79, 80, 81, 94, 96, 98, 99, 100, 103, 104, 106, 123, 125, 127, 128, 129, 132, 134, and 136 of the linear polypeptide sequence of mature hNGAL (SEQ ID NO: 1), one or more of the following mutated amino acid residues: Leu 36→Ile or Val; Ala 40→Tyr or Lys; Ile 41→Gly; Glu 44→Thr, Ile, Asp, Val, or Pro; Asp 47→Arg, Gln, or Tyr; Gln 49→Ser or Ala; Tyr 52→Phe; Leu 70→Arg; Arg 72→Glu; Lys 73→Gln, Ala, or Asn; Lys 74→Glu or Arg; Lys 75→Arg or Ser; Asp 77→His, Lys, Ser, Val, or Ile; Trp 79→Thr; Ile 80→Ser; Arg 81→Lys; Leu 94→Ala, Thr, Ser, Arg, or His; Asn 96→Ser, Tyr, or Gln; Lys 98→Gly; Ser 99→Asn; Tyr 100→Arg, Ala or His; Leu 103→Gln or Ser; Thr 104→Tyr; Tyr 106→Ser or Thr; Phe 123→Trp, His, or Ala; Lys 125→Ser, His, or Ala; Ser 127→Ile, Thr, Ala, Gln, or Arg; Gln 128→Gly or Leu; Asn 129→Thr or Ala; Tyr 132→Thr, Ser, Phe, Ile, or His; Lys 134→Ala, Val, Asn, or Phe; and Thr 136→Ala. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein preferably competes with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, a CTGF-binding hNGAL mutein according to the disclosure includes, at one or more positions corresponding to positions 28, 40, 41, 44, 47, 49, 52, 65, 70, 72, 73, 74, 75, 77, 79, 87, 94, 96, 98, 99, 100, 103, 104, 106, 125, 127, and 134 of the linear polypeptide sequence of mature hNGAL (SEQ ID NO: 1), one or more of the following mutated amino acid residues: Gln 28→His; Ala 40→Tyr; Ile 41→Gly; Glu 44→Thr; Asp 47→Arg; Gln 49→Ser; Tyr 52→Phe; Asn 65→Asp; Leu 70→Arg; Arg 72→Glu; Lys 73→Gln; Lys 74→Glu; Lys 75→Arg; Asp 77→His; Trp 79→Thr; Cys 87→Ser; Leu 94→Thr or Ser; Asn 96→Ser; Lys 98→Gly; Ser 99→Asn; Tyr 100→Arg; Leu 103→Gln or Ser; Thr 104→Tyr; Tyr 106→Ser or Thr; Lys 125→Ser; Ser 127→Ile; and Lys 134→Ala. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein is preferably competes with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, a CTGF-binding hNGAL mutein according to the disclosure includes, at one or more positions corresponding to positions 40, 41, 44, 47, 49, 52, 70, 72, 73, 74, 75, 77, 79, 94, 96, 98, 99, 100, 103, 104, 106, 125, 127, and 134 of the linear polypeptide sequence of mature hNGAL (SEQ ID NO: 1), one or more of the following mutated amino acid residues: Ala 40→Tyr; Ile 41→Gly; Glu 44→Thr; Asp 47→Arg; Gln 49→Ser; Tyr 52→Phe; Leu 70→Arg; Arg 72→Glu; Lys 73→Gln; Lys 74→Glu; Lys 75→Arg; Asp 77→His; Trp 79→Thr; Leu 94→Thr or Ser; Asn 96→Ser; Lys 98→Gly; Ser 99→Asn; Tyr 100→Arg; Leu 103→Gln or Ser; Thr 104→Tyr; Tyr 106→Ser or Thr; Lys 125→Ser; Ser 127→Ile; and Lys 134→Ala. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein preferably competes with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, a CTGF-binding hNGAL mutein according to the disclosure includes, at one or more positions corresponding to positions 28, 36, 40, 44, 47, 49, 52, 65, 70, 72, 73, 74, 75, 77, 79, 80, 81, 87, 94, 96, 100, 103, 106, 123, 125, 127, 128, 129, 132, 134, and 136 of the linear polypeptide sequence of mature hNGAL (SEQ ID NO: 1), one or more of the following mutated amino acid residues: Gln 28→His; Leu 36→Ile or Val; Ala 40→Lys; Glu 44→Thr, Ile, Asp, Val, or Pro; Asp 47→Gln or Tyr; Gln 49→Ala; Tyr 52→Phe; Asn 65→Asp; Leu 70→Arg; Arg 72→Glu; Lys 73→Ala or Asn; Lys 74→Arg; Lys 75→Ser; Asp 77→Lys, Ser, Val, or Ile; Trp 79→Thr; Ile 80→Ser; Arg 81→Lys; Cys 87→Ser; Leu 94→Ala, Thr, Ser, Arg, or His; Asn 96→Tyr and Gln; Tyr 100→Ala or His; Leu 103→Gln; Tyr 106→Thr; Phe 123→Trp, His, or Ala; Lys 125→Ser, His, or Ala; Ser 127→Thr, Ala, Gln, or Arg; Gln 128→Gly or Leu; Asn 129→Thr or Ala; Tyr 132→Thr, Ser, Phe, Ile, or His; Lys 134→Val, Asn, or Phe; and Thr 136→Ala. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein preferably competes with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, a CTGF-binding hNGAL mutein according to the disclosure includes, at one or more positions corresponding to positions 36, 40, 44, 47, 49, 52, 70, 72, 73, 74, 75, 77, 79, 80, 81, 94, 96, 100, 103, 106, 123, 125, 127, 128, 129, 132, 134, and 136 of the linear polypeptide sequence of mature hNGAL (SEQ ID NO: 1), one or more of the following mutated amino acid residues: Leu 36→Ile or Val; Ala 40→Lys; Glu 44→Thr, Ile, Asp, Val, or Pro; Asp 47→Gln or Tyr; Gln 49→Ala; Tyr 52→Phe; Leu 70→Arg; Arg 72→Glu; Lys 73→Ala or Asn; Lys 74→Arg; Lys 75→Ser; Asp 77→Lys, Ser, Val, or Ile; Trp 79→Thr; Ile 80→Ser; Arg 81→Lys; Leu 94→Ala, Thr, Ser, Arg, or His; Asn 96→Tyr and Gln; Tyr 100→Ala or His; Leu 103→Gln; Tyr 106→Thr; Phe 123→Trp, His, or Ala; Lys 125→Ser, His, or Ala; Ser 127→Thr, Ala, Gln, or Arg; Gln 128→Gly or Leu; Asn 129→Thr or Ala; Tyr 132→Thr, Ser, Phe, Ile, or His; Lys 134→Val, Asn, or Phe; and Thr 136→Ala. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein preferably competes with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, a CTGF-binding hNGAL mutein according to the disclosure includes, at one or more positions corresponding to positions 28, 36, 40, 41, 47, 49, 52, 65, 68, 70, 72, 73, 77, 79, 81, 87, 94, 95, 96, 97, 98, 99, 100, 102, 103, 104, 106, 123, 127, 128, 129, 130, 132, 134, and 136 of the linear polypeptide sequence of mature hNGAL (SEQ ID NO: 1), one or more of the following mutated amino acid residues: Gln 28→His; Leu 36→Met or Trp; Ala 40→Phe, Tyr, Ile, or Val; Ile 41→Arg or Lys; Asp 47→Gln; Gln 49→Ser, Phe, Leu, or Ala; Tyr 52→Gly or Ser; Asn 65→Asp; Ser 68→Gln or Glu; Leu 70→Gln or Val; Arg 72→Asp or Glu; Lys 73→Asp or Gln; Asp 77→Leu or His; Trp 79→Val or Ile; Arg 81→Glu or Lys; Cys 87→Ser; Leu 94→Ala or Glu; Gly 95→Ser; Asn 96→Ala, Asp, or Pro; Ile 97→Tyr; Lys 98→Ser; Ser 99→Val or Arg; Tyr 100→Phe, Arg, Pro, or Ser; Gly 102→Thr or Arg; Leu 103→Phe, Glu or Tyr; Thr 104→Glu, Val, or Trp; Tyr 106→Gln, Ser, Thr, His, or Asp; Phe 123→Leu or Val; Ser 127→Tyr, Trp, Phe, His, or Gly; Gln 128→Gly or Pro; Asn 129→Ser; Arg 130→Glu or Leu; Tyr 132→Val or Phe; Lys 134→Trp, His, or Gln; and Thr 136→Val. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein preferably competes with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, a CTGF-binding hNGAL mutein according to the disclosure includes, at one or more positions corresponding to positions 36, 40, 41, 47, 49, 52, 68, 70, 72, 73, 77, 79, 81, 94, 95, 96, 97, 98, 99, 100, 102, 103, 104, 106, 123, 127, 128, 129, 130, 132, 134, and 136 of the linear polypeptide sequence of mature hNGAL (SEQ ID NO: 1), one or more of the following mutated amino acid residues: Leu 36→Met or Trp; Ala 40→Phe, Tyr, Ile, or Val; Ile 41→Arg or Lys; Asp 47→Gln; Gln 49→Ser, Phe, Leu, or Ala; Tyr 52→Gly or Ser; Ser 68→Gln or Glu; Leu 70→Gln or Val; Arg 72→Asp or Glu; Lys 73→Asp or Gln; Asp 77→Leu or His; Trp 79→Val or Ile; Arg 81→Glu or Lys; Leu 94→Ala or Glu; Gly 95→Ser; Asn 96→Ala, Asp, or Pro; Ile 97→Tyr; Lys 98→Ser; Ser 99→Val or Arg; Tyr 100→Phe, Arg, Pro, or Ser; Gly 102→Thr or Arg; Leu 103→Phe, Glu or Tyr; Thr 104→Glu, Val, or Trp; Tyr 106→Gln, Ser, Thr, His, or Asp; Phe 123→Leu or Val; Ser 127→Tyr, Trp, Phe, His, or Gly; Gln 128→Gly or Pro; Asn 129→Ser; Arg 130→Glu or Leu; Tyr 132→Val or Phe; Lys 134→Trp, His, or Gln; and Thr 136→Val. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein preferably competes with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, a CTGF-binding hNGAL mutein according to the disclosure includes, at one or more positions corresponding to positions 28, 36, 40, 41, 49, 52, 65, 68, 70, 72, 73, 77, 79, 81, 87, 96, 99, 100, 102, 103, 104, 106, 123, 127, 128, 129, 130, 132, 134, and 136 of the linear polypeptide sequence of mature hNGAL (SEQ ID NO: 1), one or more of the following mutated amino acid residues: Gln 28→His; Leu 36→Met; Ala 40→Phe or Tyr; Ile 41→Arg; Gln 49→Ser; Tyr 52→Gly; Asn 65→Asp; Ser 68→Gln; Leu 70→Gln; Arg 72→Asp; Lys 73→Asp; Asp 77→Leu; Trp 79→Val; Arg 81→Glu; Cys 87→Ser; Asn 96→Ala; Ser 99→Val; Tyr 100→Phe; Gly 102→Thr; Leu 103→Phe; Thr 104→Glu; Tyr 106→Gln; Phe 123→Leu or Val; Ser 127→Tyr or Trp; Gln 128→Gly or Pro; Asn 129→Ser; Arg 130→Glu or Leu; Tyr 132→Val; Lys 134→Trp, His, or Gln; and Thr 136→Val. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein preferably does not compete with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, a CTGF-binding hNGAL mutein according to the disclosure includes, at one or more positions corresponding to positions 36, 40, 41, 49, 52, 68, 70, 72, 73, 77, 79, 81, 96, 99, 100, 102, 103, 104, 106, 123, 127, 128, 129, 130, 132, 134, and 136 of the linear polypeptide sequence of mature hNGAL (SEQ ID NO: 1), one or more of the following mutated amino acid residues: Leu 36→Met; Ala 40→Phe or Tyr; Ile 41→Arg; Gln 49→Ser; Tyr 52→Gly; Ser 68→Gln; Leu 70→Gln; Arg 72→Asp; Lys 73→Asp; Asp 77→Leu; Trp 79→Val; Arg 81→Glu; Asn 96→Ala; Ser 99→Val; Tyr 100→Phe; Gly 102→Thr; Leu 103→Phe; Thr 104→Glu; Tyr 106→Gln; Phe 123→Leu or Val; Ser 127→Tyr or Trp; Gln 128→Gly or Pro; Asn 129→Ser; Arg 130→Glu or Leu; Tyr 132→Val; Lys 134→Trp, His, or Gln; and Thr 136→Val. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein preferably does not compete with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, a CTGF-binding hNGAL mutein according to the disclosure includes, at one or more positions corresponding to positions 28, 36, 40, 41, 47, 49, 52, 65, 68, 70, 72, 73, 77, 79, 81, 87, 94, 95, 96, 97, 98, 99, 100, 102, 103, 104, 106, 127, 132, and 134 of the linear polypeptide sequence of mature hNGAL (SEQ ID NO: 1), one or more of the following mutated amino acid residues: Gln 28→His; Leu 36→Trp; Ala 40→Ile or Val; Ile 41→Lys; Asp 47→Gln; Gln 49→Phe, Leu, or Ala; Tyr 52→Ser; Asn 65→Asp; Ser 68→Glu; Leu 70→Val; Arg 72→Glu; Lys 73→Gln; Asp 77→His; Trp 79→Ile; Arg 81→Lys; Cys 87→Ser; Leu 94→Ala or Glu; Gly 95→Ser; Asn 96→Asp or Pro; Ile 97→Tyr; Lys 98→Ser; Ser 99→Arg; Tyr 100→Arg, Pro, or Ser; Gly 102→Arg; Leu 103→Glu or Tyr; Thr 104→Val or Trp; Tyr 106→Ser, Thr, His, or Asp; Ser 127→Phe, His, or Gly; Tyr 132→Phe; and Lys 134→Trp. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein preferably does not compete with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, a CTGF-binding hNGAL mutein according to the disclosure includes, at one or more positions corresponding to positions 36, 40, 41, 47, 49, 52, 68, 70, 72, 73, 77, 79, 81, 94, 95, 96, 97, 98, 99, 100, 102, 103, 104, 106, 127, 132, and 134 of the linear polypeptide sequence of mature hNGAL (SEQ ID NO: 1), one or more of the following mutated amino acid residues: Leu 36→Trp; Ala 40→Ile or Val; Ile 41→Lys; Asp 47→Gln; Gln 49→Phe, Leu, or Ala; Tyr 52→Ser; Ser 68→Glu; Leu 70→Val; Arg 72→Glu; Lys 73→Gln; Asp 77→His; Trp 79→Ile; Arg 81→Lys; Leu 94→Ala or Glu; Gly 95→Ser; Asn 96→Asp or Pro; Ile 97→Tyr; Lys 98→Ser; Ser 99→Arg; Tyr 100→Arg, Pro, or Ser; Gly 102→Arg; Leu 103→Glu or Tyr; Thr 104→Val or Trp; Tyr 106→Ser, Thr, His, or Asp; Ser 127→Phe, His, or Gly; Tyr 132→Phe; and Lys 134→Trp. In some embodiments, an hNGAL mutein according to the disclosure includes two or more, such as 3, 4, 5, 6, 7, 8, 9, 10, 11, 12, 13, 14, 15, 16, 17, 18, 19, 20, 21, 22, 23, 24, or more mutated amino acid residues at these sequence positions of mature hNGAL (SEQ ID NO: 1). In some embodiments, an hNGAL mutein of the disclosure comprises 10 or more of the above-mentioned mutated amino acid residues at these sequence positions of mature hNGAL (SEQ ID NO: 1). In some embodiments, an hNGAL mutein of the disclosure comprises 15 or more of the above-mentioned mutated amino acid residues at these sequence positions of mature hNGAL (SEQ ID NO: 1). In some embodiments, an hNGAL mutein of the disclosure comprises 20 or more of the above-mentioned mutated amino acid residues at these sequence positions of mature hNGAL (SEQ ID NO: 1). Such an hNGAL mutein preferably does not compete with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, the CTGF-binding hNGAL muteins include one of the following sets of mutated amino acid residues in comparison with the linear polypeptide sequence of mature hNGAL (SEQ ID NO: 1):
(a) Gln 28→His, Leu 36→Arg, Ala 40→Asn, Ile 41→Arg, Gln 49→Pro, Tyr 52→Trp, Ser 68→His, Leu 70→His, Arg 72→Met, Lys 73→Thr, Asp 77→Arg, Trp 79→Ile, Arg 81→Asp, Cys 87→Ser, Asn 96→Ala, Tyr 100→Gly, Leu 103→Met, Tyr 106→Pro, Val 110→Ile, Lys 125→Trp, Ser 127→Asn, Tyr 132→Trp, and Lys 134→Thr;
(b) Gln 28→His, Leu 36→Arg, Ala 40→Asn, Ile 41→Arg, Gln 49→Pro, Tyr 52→Trp, Asn 65→Asp, Ser 68→His, Leu 70→His, Arg 72→Met, Lys 73→Thr, Asp 77→Arg, Trp 79→Ile, Arg 81→Asp, Cys 87→Ser, Asn 96→Ala, Tyr 100→Gly, Leu 103→Met, Tyr 106→Pro, Val 110→Ile, Lys 125→Trp, Ser 127→Asn, Tyr 132→Trp, and Lys 134→Thr;
(c) Leu 36→Lys, Ile 41→deletion of Ile 41, Asp 47→Glu, Gln 49→Pro, Tyr 52→Trp, Asn 65→Asp, Ser 68→His, Leu 70→His, Arg 72→Leu, Lys 73→Thr, Asp 77→Lys, Trp 79→Leu, Arg 81→Asp, Cys 87→Ser, Leu 94→Ile, Asn 96→Ala, Tyr 100→Gly, Tyr 106→Pro, Lys 125→Trp, Tyr 132→Trp, and Lys 134→Thr; or
(d) Leu 36→Arg, Ala 40→Asn, Ile 41→Gln, Asp 47→Ser, Gln 49→Pro, Tyr 52→Trp, Asn 65→Asp, Ser 68→His, Leu 70→His, Arg 72→Ser, Lys 73→Thr, Asp 77→Arg, Trp 79→Ile, Arg 81→Asp, Cys 87→Ser, Leu 94→Ala, Asn 96→Ala, Tyr 100→Gly, Tyr 106→Pro, Lys 125→Trp, Ser 127→Thr, Tyr 132→Trp, and Lys 134→Thr.
   In some embodiments, the CTGF-binding hNGAL muteins include all but three, all but two, or all but one mutated amino acid residues of one of the aforementioned sets of mutated amino acid residues in comparison with the linear polypeptide sequence of mature hNGAL (SEQ ID NO: 1). Such an hNGAL mutein preferably does not compete with the antibody shown in SEQ ID NOs: 60 and 61 for CTGF binding, e.g., as determined in an SPR assay, such as essentially described in Example 8. Such an hNGAL mutein preferably does not bind to a fragment comprising only domains 1 and 2 of CTGF, while being capable of binding to full-length CTGF, e.g., as determined in an ELISA assay, such as essentially described in Example 9.

In some embodiments, the CTGF-binding hNGAL muteins include one of the following sets of mutated amino acid residues in comparison with the linear polypeptide sequence of mature hNGAL (SEQ ID NO: 1):
(e) Gln 28→His, Ala 40→Tyr, Ile 41→Gly, Glu 44→Thr, Asp 47→Arg, Gln 49→Ser, Tyr 52→Phe, Asn 65→Asp, Leu 70→Arg, Arg 72→Glu, Lys 73→Gln, Lys 74→Glu, Lys 75→Arg, Asp 77→His, Trp 79→Thr, Cys 87→Ser, Leu 94→Thr, Asn 96→Ser, Tyr 100→Arg, Leu 103→Gln, Tyr 106→Ser, Lys 125→Ser, Ser 127→Ile, and Lys 134→Ala; or
(f) Ala 40→Tyr, Ile 41→Gly, Glu 44→Thr, Asp 47→Arg, Gln 49→Ser, Tyr 52→Phe, Asn 65→Asp, Leu 70→Arg, Arg 72→Glu, Lys 73→Gln, Lys 74→Glu, Lys 75→Arg, Asp 77→His, Trp 79→Thr, Cys 87→Ser, Leu 94→Ser, Lys 98→Gly, Ser 99→Asn, Leu 103→Ser, Thr 104→Tyr, Tyr 106→Thr, Lys 125→Ser, Ser 127→Ile, and Lys 134→Ala.
   In some embodiments, the CTGF-binding hNGAL muteins include all but three, all but two, or all but one mutated amino acid residues of one of the aforementioned sets of mutated amino acid residues in comparison with the linear polypeptide sequence of mature hNGAL (SEQ ID NO: 1). Such an hNGAL mutein preferably competes with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, the CTGF-binding hNGAL muteins include one of the following sets of mutated amino acid residues in comparison with the linear polypeptide sequence of mature hNGAL (SEQ ID NO: 1):
(g) Gln 28→His, Ala 40→Lys, Glu 44→Ile, Asp 47→Gln, Gln 49→Ala, Tyr 52→Phe, Asn 65→Asp, Leu 70→Arg, Arg 72→Glu, Lys 73→Ala, Lys 74→Arg, Lys 75→Ser, Asp 77→Ser, Trp 79→Thr, Arg 81→Lys, Cys 87→Ser, Leu 94→Ala, Tyr 100→Ala, Leu 103→Gln, Tyr 106→Thr, Lys 125→His, Tyr 132→Thr, and Lys 134→Val;
(h) Asp 47→Gln, Gln 49→Ala, Tyr 52→Phe, Asn 65→Asp, Leu 70→Arg, Arg 72→Glu, Lys 73→Ala, Lys 75→Ser, Asp 77→Ser, Trp 79→Thr, Arg 81→Lys, Cys 87→Ser, Leu 94→Ala, Tyr 100→Ala, Leu 103→Gln, Tyr 106→Thr, Phe 123→Trp, Lys 125→Ser, Ser 127→Ala, Gln 128→Gly, Asn 129→Thr, Tyr 132→Ser, and Lys 134→Asn, Thr 136→Ala;
(i) Asp 47→Gln, Gln 49→Ala, Tyr 52→Phe, Asn 65→Asp, Leu 70→Arg, Arg 72→Glu, Lys 73→Ala, Lys 75→Ser, Asp 77→Ser, Trp 79→Thr, Ile 80→Ser, Arg 81→Lys, Cys 87→Ser, Leu 94→Ala, Tyr 100→Ala, Leu 103→Gln, Tyr 106→Thr, Lys 125→His, Tyr 132→Thr, and Lys 134→Val;
(j) Asp 47→Tyr, Gln 49→Ala, Tyr 52→Phe, Asn 65→Asp, Leu 70→Arg, Arg 72→Glu, Lys 73→Asn, Lys 75→Ser, Asp 77→Val, Trp 79→Thr, Arg 81→Lys, Cys 87→Ser, Leu 94→Thr, Tyr 100→Ala, Leu 103→Gln, Tyr 106→Thr, Lys 125→His, Tyr 132→Phe, and Lys 134→Val;
(k) Asp 47→Tyr, Gln 49→Ala, Tyr 52→Phe, Asn 65→Asp, Leu 70→Arg, Arg 72→Glu, Lys 73→Asn, Lys 75→Ser, Asp 77→Val, Trp 79→Thr, Ile 80→Ser, Arg 81→Lys, Cys 87→Ser, Leu 94→Thr, Tyr 100→Ala, Leu 103→Gln, Tyr 106→Thr, Lys 125→His, Tyr 132→Phe, and Lys 134→Val;
(l) Asp 47→Tyr, Gln 49→Ala, Tyr 52→Phe, Asn 65→Asp, Leu 70→Arg, Arg 72→Glu, Lys 73→Ala, Lys 75→Ser, Asp 77→Ile, Trp 79→Thr, Arg 81→Lys, Cys 87→Ser, Leu 94→Arg, Tyr 100→Ala, Leu 103→Gln, Tyr 106→Thr, Lys 125→His, Tyr 132→Ile, and Lys 134→Val;
(m) Asp 47→Gln, Gln 49→Ala, Tyr 52→Phe, Asn 65→Asp, Leu 70→Arg, Arg 72→Glu, Lys 73→Ala, Lys 75→Ser, Asp 77→Lys, Trp 79→Thr, Arg 81→Lys, Cys 87→Ser, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val;
(n) Glu 44→Thr, Asp 47→Gln, Gln 49→Ala, Tyr 52→Phe, Asn 65→Asp, Leu 70→Arg, Arg 72→Glu, Lys 73→Ala, Lys 75→Ser, Asp 77→Val, Trp 79→Thr, Arg 81→Lys, Cys 87→Ser, Leu 94→Ala, Tyr 100→Ala, Leu 103→Gln, Tyr 106→Thr, Lys 125→His, Tyr 132→Phe, and Lys 134→Val;
(o) Glu 44→Thr, Asp 47→Gln, Gln 49→Ala, Tyr 52→Phe, Asn 65→Asp, Leu 70→Arg, Arg 72→Glu, Lys 73→Ala, Lys 75→Ser, Asp 77→Val, Trp 79→Thr, Ile 80→Ser, Arg 81→Lys, Cys 87→Ser, Leu 94→Ala, Tyr 100→Ala, Leu 103→Gln, Tyr 106→Thr, Lys 125→His, Tyr 132→Phe, and Lys 134→Val;
(p) Leu 36→Ile, Glu 44→Asp, Asp 47→Tyr, Gln 49→Ala, Tyr 52→Phe, Asn 65→Asp, Leu 70→Arg, Arg 72→Glu, Lys 73→Asn, Lys 75→Ser, Asp 77→Ile, Trp 79→Thr, Arg 81→Lys, Cys 87→Ser, Leu 94→His, Tyr 100→His, Leu 103→Gln, Tyr 106→Thr, Lys 125→His, Tyr 132→Ile, and Lys 134→Val;
(q) Asp 47→Gln, Gln 49→Ala, Tyr 52→Phe, Asn 65→Asp, Leu 70→Arg, Arg 72→Glu, Lys 73→Ala, Lys 75→Ser, Asp 77→Ser, Trp 79→Thr, Arg 81→Lys, Cys 87→Ser, Leu 94→Ala, Tyr 100→Ala, Leu 103→Gln, Tyr 106→Thr, Phe 123→His, Lys 125→Ala, Tyr 132→Ser, and Lys 134→Val;
(r) Asp 47→Gln, Gln 49→Ala, Tyr 52→Phe, Asn 65→Asp, Leu 70→Arg, Arg 72→Glu, Lys 73→Ala, Lys 75→Ser, Asp 77→Ser, Trp 79→Thr, Arg 81→Lys, Cys 87→Ser, Leu 94→Ala, Tyr 100→Ala, Leu 103→Gln, Tyr 106→Thr, Phe 123→Ala, Lys 125→Ala, Ser 127→Gln, Gln 128→Leu, Tyr 132→His, and Lys 134→Phe;
(s) Asp 47→Gln, Gln 49→Ala, Tyr 52→Phe, Asn 65→Asp, Leu 70→Arg, Arg 72→Glu, Lys 73→Ala, Lys 75→Ser, Asp 77→Ser, Trp 79→Thr, Arg 81→Lys, Cys 87→Ser, Leu 94→Ala, Tyr 100→Ala, Leu 103→Gln, Tyr 106→Thr, Lys 125→Ala, Ser 127→Arg, Gln 128→Gly, Asn 129→Ala, Tyr 132→Ser, and Lys 134→Asn, Thr 136→Ala;
(t) Leu 36→Ile, Glu 44→Val, Asp 47→Gln, Gln 49→Ala, Tyr 52→Phe, Asn 65→Asp, Leu 70→Arg, Arg 72→Glu, Lys 73→Ala, Lys 75→Ser, Asp 77→Val, Trp 79→Thr, Arg 81→Lys, Cys 87→Ser, Leu 94→Ala, Tyr 100→Ala, Leu 103→Gln, Tyr 106→Thr, Lys 125→His, Tyr 132→Ile, and Lys 134→Val;
(u) Leu 36→Val, Glu 44→Pro, Asp 47→Gln, Gln 49→Ala, Tyr 52→Phe, Asn 65→Asp, Leu 70→Arg, Arg 72→Glu, Lys 73→Ala, Lys 75→Ser, Asp 77→Lys, Trp 79→Thr, Arg 81→Lys, Cys 87→Ser, Leu 94→Ser, Asn 96→Gln, Tyr 100→Ala, Leu 103→Gln, Tyr 106→Thr, Lys 125→His, Tyr 132→Ile, and Lys 134→Val; or
(v) Leu 36→Val, Glu 44→Thr, Asp 47→Gln, Gln 49→Ala, Tyr 52→Phe, Asn 65→Asp, Leu 70→Arg, Arg 72→Glu, Lys 73→Ala, Lys 75→Ser, Asp 77→Lys, Trp 79→Thr, Arg 81→Lys, Cys 87→Ser, Leu 94→Ala, Tyr 100→Ala, Leu 103→Gln, Tyr 106→Thr, Lys 125→His, Tyr 132→Ile, and Lys 134→Val.
   In some embodiments, the CTGF-binding hNGAL muteins include all but three, all but two, or all but one mutated amino acid residues of one of the aforementioned sets of mutated amino acid residues in comparison with the linear polypeptide sequence of mature hNGAL (SEQ ID NO: 1). Such an hNGAL mutein preferably competes with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, the CTGF-binding hNGAL muteins include one of the following sets of mutated amino acid residues in comparison with the linear polypeptide sequence of mature hNGAL (SEQ ID NO: 1):
(w) Gln 28→His, Leu 36→Met, Ala 40→Phe, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Cys 87→Ser, Asn 96→Ala, Tyr 100→Phe, Leu 103→Phe, Tyr 106→Gln, Ser 127→Tyr, Tyr 132→Val, and Lys 134→Trp;
(x) Ala 40→Tyr, Ile 41→Arg, Gln 49→Ser, Tyr 52→Gly, Asn 65→Asp, Ser 68→Gln, Leu 70→Gln, Arg 72→Asp, Lys 73→Asp, Asp 77→Leu, Trp 79→Val, Arg 81→Glu, Cys 87→Ser, Ser 99→Val, Gly 102→Thr, Thr 104→Glu, Tyr 106→Gln, Ser 127→Tyr, Tyr 132→Val, and Lys 134→Trp;
(y) Ala 40→Tyr, Ile 41→Arg, Gln 49→Ser, Tyr 52→Gly, Asn 65→Asp, Ser 68→Gln, Leu 70→Gln, Arg 72→Asp, Lys 73→Asp, Asp 77→Leu, Trp 79→Val, Arg 81→Glu, Cys 87→Ser, Asn 96→Ala, Tyr 106→Gln, Phe 123→Leu, Ser 127→Tyr, Gln 128→Gly, Asn 129→Ser, Arg 130→Glu, and Lys 134→His; or
(z) Ala 40→Tyr, Ile 41→Arg, Gln 49→Ser, Tyr 52→Gly, Asn 65→Asp, Ser 68→Gln, Leu 70→Gln, Arg 72→Asp, Lys 73→Asp, Asp 77→Leu, Trp 79→Val, Arg 81→Glu, Cys 87→Ser, Asn 96→Ala, Tyr 106→Gln, Phe 123→Val, Ser 127→Trp, Gln 128→Pro, Arg 130→Leu, and Lys 134→Gln, Thr 136→Val.
   In some embodiments, the CTGF-binding hNGAL muteins include all but three, all but two, or all but one mutated amino acid residues of one of the aforementioned sets of mutated amino acid residues in comparison with the linear polypeptide sequence of mature hNGAL (SEQ ID NO: 1). Such an hNGAL mutein preferably does not compete with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, the CTGF-binding hNGAL muteins include one of the following sets of mutated amino acid residues in comparison with the linear polypeptide sequence of mature hNGAL (SEQ ID NO: 1):
(aa) Gln 28→His, Leu 36→Trp, Ala 40→Ile, Ile 41→Lys, Gln 49→Phe, Tyr 52→Ser, Ser 68→Glu, Leu 70→Val, Arg 72→Glu, Lys 73→Gln, Asp 77→His, Trp 79→Ile, Arg 81→Lys, Cys 87→Ser, Asn 96→Asp, Tyr 100→Pro, Leu 103→Glu, Tyr 106→Ser, Ser 127→Phe, Tyr 132→Phe, and Lys 134→Trp;
(bb) Gln 28→His, Leu 36→Trp, Ala 40→Ile, Ile 41→Lys, Gln 49→Phe, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Asn 96→Asp, Tyr 100→Pro, Leu 103→Glu, Tyr 106→Ser, Ser 127→Phe, Tyr 132→Phe, and Lys 134→Trp;
(cc) Leu 36→Trp, Ala 40→Val, Ile 41→Lys, Asp 47→Gln, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Leu 94→Ala, Asn 96→Asp, Tyr 100→Pro, Leu 103→Tyr, Tyr 106→Ser, Tyr 132→Phe, and Lys 134→Trp;
(dd) Leu 36→Trp, Ala 40→Val, Ile 41→Lys, Asp 47→Gln, Gln 49→Phe, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Gly 95→Ser, Asn 96→Pro, Lys 98→Ser, Tyr 100→Ser, Thr 104→Val, Tyr 106→His, Ser 127→His, Tyr 132→Phe, and Lys 134→Trp;
(ee) Leu 36→Trp, Ile 41→Lys, Asp 47→Gln, Gln 49→Phe, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Leu 94→Glu, Ile 97→Tyr, Ser 99→Arg, Tyr 100→Arg, Gly 102→Arg, Thr 104→Trp, Tyr 106→Asp, Ser 127→His, Tyr 132→Phe, and Lys 134→Trp;
(ff) Leu 36→Trp, Ala 40→Val, Asp 47→Gln, Gln 49→Phe, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Asn 96→Asp, Tyr 100→Pro, Leu 103→Glu, Tyr 106→Thr, Tyr 132→Phe, and Lys 134→Trp;
(gg) Leu 36→Trp, Ala 40→Val, Ile 41→Lys, Asp 47→Gln, Gln 49→Leu, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Leu 94→Ala, Asn 96→Asp, Tyr 100→Pro, Leu 103→Tyr, Tyr 106→Ser, Tyr 132→Phe, and Lys 134→Trp; or
(hh) Leu 36→Trp, Ala 40→Val, Ile 41→Lys, Asp 47→Gln, Gln 49→Ala, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Leu 94→Ala, Asn 96→Asp, Tyr 100→Pro, Leu 103→Tyr, Tyr 106→Ser, Ser 127→Gly, Tyr 132→Phe, and Lys 134→Trp.
   In some embodiments, the CTGF-binding hNGAL muteins include all but three, all but two, or all but one mutated amino acid residues of one of the aforementioned sets of mutated amino acid residues in comparison with the linear polypeptide sequence of mature hNGAL (SEQ ID NO: 1). Such an hNGAL mutein preferably does not compete with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, the CTGF-binding hNGAL muteins include one of the following sets of mutated amino acid residues in comparison with the linear polypeptide sequence of mature hNGAL (SEQ ID NO: 1):
(a) Leu 36→Arg, Ala 40→Asn, Ile 41→Arg, Gln 49→Pro, Tyr 52→Trp, Ser 68→His, Leu 70→His, Arg 72→Met, Lys 73→Thr, Asp 77→Arg, Trp 79→Ile, Arg 81→Asp, Asn 96→Ala, Tyr 100→Gly, Leu 103→Met, Tyr 106→Pro, Val 110→Ile, Lys 125→Trp, Ser 127→Asn, Tyr 132→Trp, and Lys 134→Thr;
(b) Leu 36→Arg, Ala 40→Asn, Ile 41→Arg, Gln 49→Pro, Tyr 52→Trp, Ser 68→His, Leu 70→His, Arg 72→Met, Lys 73→Thr, Asp 77→Arg, Trp 79→Ile, Arg 81→Asp, Asn 96→Ala, Tyr 100→Gly, Leu 103→Met, Tyr 106→Pro, Val 110→Ile, Lys 125→Trp, Ser 127→Asn, Tyr 132→Trp, and Lys 134→Thr;
(c) Leu 36→Lys, Ile 41→deletion of Ile 41, Asp 47→Glu, Gln 49→Pro, Tyr 52→Trp, Ser 68→His, Leu 70→His, Arg 72→Leu, Lys 73→Thr, Asp 77→Lys, Trp 79→Leu, Arg 81→Asp, Leu 94→Ile, Asn 96→Ala, Tyr 100→Gly, Tyr 106→Pro, Lys 125→Trp, Tyr 132→Trp, and Lys 134→Thr; or
(d) Leu 36→Arg, Ala 40→Asn, Ile 41→Gln, Asp 47→Ser, Gln 49→Pro, Tyr 52→Trp, Ser 68→His, Leu 70→His, Arg 72→Ser, Lys 73→Thr, Asp 77→Arg, Trp 79→Ile, Arg 81→Asp, Leu 94→Ala, Asn 96→Ala, Tyr 100→Gly, Tyr 106→Pro, Lys 125→Trp, Ser 127→Thr, Tyr 132→Trp, and Lys 134→Thr.
   In some embodiments, the CTGF-binding hNGAL muteins include all but three, all but two, or all but one mutated amino acid residues of one of the aforementioned sets of mutated amino acid residues in comparison with the linear polypeptide sequence of mature hNGAL (SEQ ID NO: 1). Such an hNGAL mutein preferably does not compete with the antibody shown in SEQ ID NOs: 60 and 61 for CTGF binding, e.g., as determined in an SPR assay, such as essentially described in Example 8. Such an hNGAL mutein preferably does not bind to a fragment comprising only domains 1 and 2 of CTGF, while being capable of binding to full-length CTGF, e.g., as determined in an ELISA assay, such as essentially described in Example 9.

In some embodiments, the CTGF-binding hNGAL muteins include one of the following sets of mutated amino acid residues in comparison with the linear polypeptide sequence of mature hNGAL (SEQ ID NO: 1):
(e) Ala 40→Tyr, Ile 41→Gly, Glu 44→Thr, Asp 47→Arg, Gln 49→Ser, Tyr 52→Phe, Leu 70→Arg, Arg 72→Glu, Lys 73→Gln, Lys 74→Glu, Lys 75→Arg, Asp 77→His, Trp 79→Thr, Leu 94→Thr, Asn 96→Ser, Tyr 100→Arg, Leu 103→Gln, Tyr 106→Ser, Lys 125→Ser, Ser 127→Ile, and Lys 134→Ala; or
(f) Ala 40→Tyr, Ile 41→Gly, Glu 44→Thr, Asp 47→Arg, Gln 49→Ser, Tyr 52→Phe, Leu 70→Arg, Arg 72→Glu, Lys 73→Gln, Lys 74→Glu, Lys 75→Arg, Asp 77→His, Trp 79→Thr, Leu 94→Ser, Lys 98→Gly, Ser 99→Asn, Leu 103→Ser, Thr 104→Tyr, Tyr 106→Thr, Lys 125→Ser, Ser 127→Ile, and Lys 134→Ala.

In some embodiments, the CTGF-binding hNGAL muteins include all but three, all but two, or all but one mutated amino acid residues of one of the aforementioned sets of mutated amino acid residues in comparison with the linear polypeptide sequence of mature hNGAL (SEQ ID NO: 1). Such an hNGAL mutein preferably competes with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, the CTGF-binding hNGAL muteins include one of the following sets of mutated amino acid residues in comparison with the linear polypeptide sequence of mature hNGAL (SEQ ID NO: 1):
(g) Ala 40→Lys, Glu 44→Ile, Asp 47→Gln, Gln 49→Ala, Tyr 52→Phe, Leu 70→Arg, Arg 72→Glu, Lys 73→Ala, Lys 74→Arg, Lys 75→Ser, Asp 77→Ser, Trp 79→Thr, Arg 81→Lys, Leu 94→Ala, Tyr 100→Ala, Leu 103→Gln, Tyr 106→Thr, Lys 125→His, Tyr 132→Thr, and Lys 134→Val;
(h) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Arg 81→Lys, Leu 94→Ala, Tyr 100→Ala, Leu 103→Gln, Tyr 106→Thr, Phe 123→Trp, Lys 125→Ser, Ser 127→Ala, Gln 128→Gly, Asn 129→Thr, Tyr 132→Ser, and Lys 134→Asn, Thr 136→Ala;
(i) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Ile 80→Ser, Arg 81→Lys, Leu 94→Ala, Tyr 100→Ala, Leu 103→Gln, Tyr 106→Thr, Lys 125→His, Tyr 132→Thr, and Lys 134→Val;
(j) Asp 47→Tyr, Gln 49→Ala, Tyr 52→Phe, Leu 70→Arg, Arg 72→Glu, Lys 73→Asn, Lys 75→Ser, Asp 77→Val, Trp 79→Thr, Arg 81→Lys, Leu 94→Thr, Tyr 100→Ala, Leu 103→Gln, Tyr 106→Thr, Lys 125→His, Tyr 132→Phe, and Lys 134→Val;
(k) Asp 47→Tyr, Gln 49→Ala, Tyr 52→Phe, Leu 70→Arg, Arg 72→Glu, Lys 73→Asn, Lys 75→Ser, Asp 77→Val, Trp 79→Thr, Ile 80→Ser, Arg 81→Lys, Leu 94→Thr, Tyr 100→Ala, Leu 103→Gln, Tyr 106→Thr, Lys 125→His, Tyr 132→Phe, and Lys 134→Val;
(l) Asp 47→Tyr, Gln 49→Ala, Tyr 52→Phe, Leu 70→Arg, Arg 72→Glu, Lys 73→Ala, Lys 75→Ser, Asp 77→Ile, Trp 79→Thr, Arg 81→Lys, Leu 94→Arg, Tyr 100→Ala, Leu 103→Gln, Tyr 106→Thr, Lys 125→His, Tyr 132→Ile, and Lys 134→Val;
(m) Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val;
(n) Glu 44→Thr, Asp 47→Gln, Gln 49→Ala, Tyr 52→Phe, Leu 70→Arg, Arg 72→Glu, Lys 73→Ala, Lys 75→Ser, Asp 77→Val, Trp 79→Thr, Arg 81→Lys, Leu 94→Ala, Tyr 100→Ala, Leu 103→Gln, Tyr 106→Thr, Lys 125→His, Tyr 132→Phe, and Lys 134→Val;
(o) Glu 44→Thr, Asp 47→Gln, Gln 49→Ala, Tyr 52→Phe, Leu 70→Arg, Arg 72→Glu, Lys 73→Ala, Lys 75→Ser, Asp 77→Val, Trp 79→Thr, Ile 80→Ser, Arg 81→Lys, Leu 94→Ala, Tyr 100→Ala, Leu 103→Gln, Tyr 106→Thr, Lys 125→His, Tyr 132→Phe, and Lys 134→Val;
(p) Leu 36→Ile, Glu 44→Asp, Asp 47→Tyr, Gln 49→Ala, Tyr 52→Phe, Leu 70→Arg, Arg 72→Glu, Lys 73→Asn, Lys 75→Ser, Asp 77→Ile, Trp 79→Thr, Arg 81→Lys, Leu 94→His, Tyr 100→His, Leu 103→Gln, Tyr 106→Thr, Lys 125→His, Tyr 132→Ile, and Lys 134→Val;
(q) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Arg 81→Lys, Leu 94→Ala, Tyr 100→Ala, Leu 103→Gln, Tyr 106→Thr, Phe 123→His, Lys 125→Ala, Tyr 132→Ser, and Lys 134→Val;
(r) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Arg 81→Lys, Leu 94→Ala, Tyr 100→Ala, Leu 103→Gln, Tyr 106→Thr, Phe 123→Ala, Lys 125→Ala, Ser 127→Gln, Gln 128→Leu, Tyr 132→His, and Lys 134→Phe;
(s) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Arg 81→Lys, Leu 94→Ala, Tyr 100→Ala, Leu 103→Gln, Tyr 106→Thr, Lys 125→Ala, Ser 127→Arg, Gln 128→Gly, Asn 129→Ala, Tyr 132→Ser, and Lys 134→Asn, Thr 136→Ala;
(t) Leu 36→Ile, Glu 44→Val, Asp 47→Gln, Gln 49→Ala, Tyr 52→Phe, Leu 70→Arg, Arg 72→Glu, Lys 73→Ala, Lys 75→Ser, Asp 77→Val, Trp 79→Thr, Arg 81→Lys, Leu 94→Ala, Tyr 100→Ala, Leu 103→Gln, Tyr 106→Thr, Lys 125→His, Tyr 132→Ile, and Lys 134→Val;
(u) Leu 36→Val, Glu 44→Pro, Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ser, Asn 96→Gln, Tyr 100→Ala, Leu 103→Gln, Tyr 106→Thr, Lys 125→His, Tyr 132→Ile, and Lys 134→Val; or
(v) Leu 36→Val, Glu 44→Thr, Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Tyr 100→Ala, Leu 103→Gln, Tyr 106→Thr, Lys 125→His, Tyr 132→Ile, and Lys 134→Val.
   In some embodiments, the CTGF-binding hNGAL muteins include all but three, all but two, or all but one mutated amino acid residues of one of the aforementioned sets of mutated amino acid residues in comparison with the linear polypeptide sequence of mature hNGAL (SEQ ID NO: 1). Such an hNGAL mutein preferably competes with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, the CTGF-binding hNGAL muteins include one of the following sets of mutated amino acid residues in comparison with the linear polypeptide sequence of mature hNGAL (SEQ ID NO: 1):
(w) Leu 36→Met, Ala 40→Phe, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 100→Phe, Leu 103→Phe, Tyr 106→Gln, Ser 127→Tyr, Tyr 132→Val, and Lys 134→Trp;
(x) Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Ser 99→Val, Gly 102→Thr, Thr 104→Glu, Tyr 106→Gln, Ser 127→Tyr, Tyr 132→Val, and Lys 134→Trp;
(y) Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 106→Gln, Phe 123→Leu, Ser 127→Tyr, Gln 128→Gly, Asn 129→Ser, Arg 130→Glu, and Lys 134→His; or
(z) Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 106→Gln, Phe 123→Val, Ser 127→Trp, Gln 128→Pro, Arg 130→Leu, and Lys 134→Gln, Thr 136→Val.
   In some embodiments, the CTGF-binding hNGAL muteins include all but three, all but two, or all but one mutated amino acid residues of one of the aforementioned sets of mutated amino acid residues in comparison with the linear polypeptide sequence of mature hNGAL (SEQ ID NO: 1). Such an hNGAL mutein preferably does not compete with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In some embodiments, the CTGF-binding hNGAL muteins include one of the following sets of mutated amino acid residues in comparison with the linear polypeptide sequence of mature hNGAL (SEQ ID NO: 1):
(aa) Leu 36→Trp, Ala 40→Ile, Ile 41→Lys, Gln 49→Phe, Tyr 52→Ser, Ser 68→Glu, Leu 70→Val, Arg 72→Glu, Lys 73→Gln, Asp 77→His, Trp 79→Ile, Arg 81→Lys, Asn 96→Asp, Tyr 100→Pro, Leu 103→Glu, Tyr 106→Ser, Ser 127→Phe, Tyr 132→Phe, and Lys 134→Trp;
(bb) Leu 36→Trp, Ala 40→Ile, Ile 41→Lys, Gln 49→Phe, Tyr 52→Ser, Ser 68→Glu, Leu 70→Val, Arg 72→Glu, Lys 73→Gln, Asp 77→His, Trp 79→Ile, Arg 81→Lys, Asn 96→Asp, Tyr 100→Pro, Leu 103→Glu, Tyr 106→Ser, Ser 127→Phe, Tyr 132→Phe, and Lys 134→Trp;
(cc) Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp;
(dd) Leu 36→Trp, Ala 40→Val, Ile 41→Lys, Asp 47→Gln, Gln 49→Phe, Tyr 52→Ser, Ser 68→Glu, Leu 70→Val, Arg 72→Glu, Lys 73→Gln, Asp 77→His, Trp 79→Ile, Arg 81→Lys, Gly 95→Ser, Asn 96→Pro, Lys 98→Ser, Tyr 100→Ser, Thr 104→Val, Tyr 106→His, Ser 127→His, Tyr 132→Phe, and Lys 134→Trp;
(ee) Leu 36→Trp, Ile 41→Lys, Asp 47→Gln, Gln 49→Phe, Tyr 52→Ser, Ser 68→Glu, Leu 70→Val, Arg 72→Glu, Lys 73→Gln, Asp 77→His, Trp 79→Ile, Arg 81→Lys, Leu 94→Glu, Ile 97→Tyr, Ser 99→Arg, Tyr 100→Arg, Gly 102→Arg, Thr 104→Trp, Tyr 106→Asp, Ser 127→His, Tyr 132→Phe, and Lys 134→Trp;
(ff) Leu 36→Trp, Ala 40→Val, Asp 47→Gln, Gln 49→Phe, Tyr 52→Ser, Ser 68→Glu, Leu 70→Val, Arg 72→Glu, Lys 73→Gln, Asp 77→His, Trp 79→Ile, Arg 81→Lys, Asn 96→Asp, Tyr 100→Pro, Leu 103→Glu, Tyr 106→Thr, Tyr 132→Phe, and Lys 134→Trp;
(gg) Leu 36→Trp, Ala 40→Val, Ile 41→Lys, Asp 47→Gln, Gln 49→Leu, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp; or
(hh) Leu 36→Trp, Ala 40→Val, Ile 41→Lys, Asp 47→Gln, Gln 49→Ala, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Ser 127→Gly, Tyr 132→Phe, and Lys 134→Trp.
   In some embodiments, the CTGF-binding hNGAL muteins include all but three, all but two, or all but one mutated amino acid residues of one of the aforementioned sets of mutated amino acid residues in comparison with the linear polypeptide sequence of mature hNGAL (SEQ ID NO: 1). Such an hNGAL mutein preferably does not compete with the antibody shown in SEQ ID NOs: 60 and 61 for CTGF binding, e.g., as determined in an SPR assay, such as essentially described in Example 8. Such an hNGAL mutein is preferably capable of binding a fragment comprising only domains 1 and 2 of CTGF, e.g., as determined in an ELISA assay, such as essentially described in Example 9.

In the residual region, i.e. the region differing from sequence positions 28, 36, 40, 41, 44, 47, 49, 52, 65, 68, 70, 72, 73, 74, 75, 77, 79, 80, 81, 87, 94, 95, 96, 97, 98, 99, 100, 102, 103, 104, 106, 110, 123, 125, 127, 128, 129, 130, 132, 134, and 136, an hNGAL mutein of the disclosure may include the wild-type (natural) amino acid sequence of the linear polypeptide sequence of mature hNGAL outside the mutated amino acid sequence positions.

In still further embodiments, an hNGAL mutein according to the current disclosure has at least 70% sequence identity or at least 70% sequence homology to the sequence of mature hNGAL (SEQ ID NO: 1).

In further particular embodiments, an hNGAL mutein of the disclosure comprises an amino acid sequence as set forth in any one of SEQ ID NOs: 3-36 or a fragment or variant thereof.

In further particular embodiments, an hNGAL mutein of the disclosure has at least 75%, at least 80%, at least 85%, at least 90%, at least 95%, at least 98%, at least 99% or higher sequence identity to an amino acid sequence selected from the group consisting of SEQ ID NOs: 3-36.

The disclosure also includes structural homologues of an hNGAL mutein having an amino acid sequence selected from the group consisting of SEQ ID NOs: 3-36, which structural homologues have an amino acid sequence homology or sequence identity of more than about 60%, preferably more than 65%, more than 70%, more than 75%, more than 80%, more than 85%, more than 90%, more than 92% and most preferably more than 95% in relation to said hNGAL mutein.

In some particular embodiments, the present disclosure provides a lipocalin mutein that binds CTGF with an affinity measured by a K_{D} of about 500 nM or lower, wherein the lipocalin mutein has at least 75%, at least 80%, at least 85%, preferably at least 90%, preferably at least 95%, preferably at least 98%, preferably at least 99% identity to an amino acid sequence selected from the group consisting of SEQ ID NOs: 3-36.

### C. Modifications of the Lipocalin Muteins of the Disclosure.

In some embodiments, a lipocalin mutein of the present disclosure can comprise a heterologous amino acid sequence at its N- or C-terminus, preferably C-terminus, such as a Strep-tag II (SEQ ID NO: 41) or a cleavage site sequence for certain restriction enzymes, without affecting the biological activity (e.g., binding to its target, e.g., CTGF) of the lipocalin mutein.

In some other embodiments, further modifications of a lipocalin mutein may be introduced in order to modulate certain characteristics of the mutein, such as to improve folding stability, serum stability, protein resistance or water solubility or to reduce aggregation tendency, or to introduce new characteristics to the mutein.

For example, it is possible to mutate one or more amino acid sequence positions of a lipocalin mutein to introduce new reactive groups, for example, for the conjugation to other compounds, such as polyethylene glycol (PEG), hydroxyethyl starch (HES), biotin, peptides or proteins, or for the formation of non-naturally occurring disulfide linkages. The conjugated compound, for example, PEG or HES, can in some cases increase the serum half-life of the corresponding lipocalin mutein.

In one embodiment, the reactive group of a lipocalin mutein may occur naturally in its amino acid sequence, such as naturally occurring cysteine residues in said amino acid sequence. In some other embodiments, such reactive group may be introduced via mutagenesis. In case a reactive group is introduced via mutagenesis, one possibility is the mutation of an amino acid at the appropriate position by a cysteine residue. Exemplary possibilities of such a mutation to introduce a cysteine residue into the amino acid sequence of a hNGAL mutein include the introduction of a cysteine residue at least at one of the sequence positions that correspond to sequence positions 14, 21, 60, 84, 88, 116, 141, 145, 143, 146 or 158 of the wild-type sequence of hNGAL (SEQ ID NO: 1). The generated thiol moiety may be used to PEGylate or HESylate the mutein, for example, in order to increase the serum half-life of a respective lipocalin mutein

In another embodiment, in order to provide suitable amino acid side chains as new reactive groups for conjugating one of the above compounds to a lipocalin mutein, artificial amino acids may be introduced to the amino acid sequence of a lipocalin mutein. Generally, such artificial amino acids are designed to be more reactive and thus to facilitate the conjugation to the desired compound. Such artificial amino acids (e.g., para-acetyl-phenylalanine) may be introduced by mutagenesis, for example, using an artificial tRNA.

For several applications of the lipocalin muteins disclosed herein it may be advantageous to use them in the form of fusion proteins. In some embodiments, a lipocalin mutein of the disclosure is fused at its N-terminus or its C-terminus to a protein, a protein domain or a peptide, for instance, an antibody, an antibody fragment or variant, a signal sequence and/or an affinity tag. In some other embodiments, a lipocalin mutein of the disclosure is conjugated at its N-terminus or its C-terminus to a partner, which is a protein, a protein domain or a peptide; for instance, an antibody, an antibody fragment or variant, a signal sequence and/or an affinity tag.

Affinity tags such as the Strep-tag or Strep-tag II (Schmidt et al., J Mol Biol, 1996, 255(5):753-66), the c-myc-tag, the FLAG-tag, the His-tag or the HA-tag or proteins such as glutathione-S-transferase, which allow easy detection and/or purification of recombinant proteins, are examples of suitable fusion partners. Proteins with chromogenic or fluorescent properties such as the green fluorescent protein (GFP) or the yellow fluorescent protein (YFP) are suitable fusion partners for lipocalin muteins of the disclosure as well.

In general, it is possible to label the lipocalin muteins of the disclosure with any appropriate chemical substance or enzyme, which directly or indirectly generates a detectable compound or signal in a chemical, physical, optical, or enzymatic reaction. For example, a fluorescent or radioactive label can be conjugated to a lipocalin mutein to generate fluorescence or x-rays as detectable signal. Alkaline phosphatase, horseradish peroxidase and β-galactosidase are examples of enzyme labels (and at the same time optical labels) which catalyze the formation of chromogenic reaction products. In general, all labels commonly used for antibodies (except those exclusively used with the sugar moiety in the Fc part of immunoglobulins) can also be used for conjugation to the lipocalin muteins of the disclosure.

The lipocalin muteins of the disclosure may also be conjugated with any suitable therapeutically active agent, e.g., for the targeted delivery of such agents to a given cell, tissue or organ, or for the selective targeting of cells (e.g., tumor cells) without affecting the surrounding normal cells. Examples of such therapeutically active agents include radionuclides, toxins, small organic molecules, and therapeutic peptides (such as peptides acting as agonists/antagonists of a cell surface receptor or peptides competing for a protein binding site on a given cellular target). The lipocalin muteins of the disclosure may, however, also be conjugated with therapeutically active nucleic acids such as antisense nucleic acid molecules, small interfering RNAs, micro RNAs or ribozymes. Such conjugates can be produced by methods well known in the art.

In some embodiments, a lipocalin mutein of the disclosure may be fused or conjugated to a moiety that extends the serum half-life of the mutein (in this regard see also International Patent Publication No. WO 2006/056464, where such strategies are described with reference to muteins of human neutrophil gelatinase-associated lipocalin (hNGAL) with binding affinity for CTLA-4). The moiety that extends the serum half-life may be a PEG molecule, a HES molecule, a fatty acid molecule, such as palmitic acid (Vajo and Duckworth, Pharmacol Rev, 2000, 52(1):1-9), an Fc part of an immunoglobulin, a C_{H}3 domain of an immunoglobulin, a C_{H}4 domain of an immunoglobulin, an albumin binding peptide, an albumin binding protein, or a transferrin, to name only a few.

If PEG is used as a conjugation partner, the PEG molecule can be substituted, unsubstituted, linear, or branched. It can also be an activated polyethylene derivative. Examples of suitable compounds are PEG molecules as described in International Patent Publication No. WO 1999/64016, in U.S. Pat. No. 6,177,074, or in U.S. Pat. No. 6,403,564 in relation to interferon, or as described for other proteins such as PEG-modified asparaginase, PEG-adenosine deaminase (PEG-ADA) or PEG-superoxide dismutase (Fuertges and Abuchowski, Journal of Controlled Release, 1990, 11(1-3), 139-148). The molecular weight of such a polymer, such as polyethylene glycol, may range from about 300 to about 70,000 daltons, including, for example, polyethylene glycol with a molecular weight of about 10,000, of about 20,000, of about 30,000 or of about 40,000 daltons. Moreover, as, e.g., described in U.S. Pat. No. 6,500,930 or 6,620,413, carbohydrate oligomers and polymers such as HES can be conjugated to a mutein of the disclosure for the purpose of serum half-life extension.

If an Fc part of an immunoglobulin is used for the purpose to prolong the serum half-life of the lipocalin muteins of the disclosure, the SynFusion™ technology, commercially available from Syntonix Pharmaceuticals, Inc. (MA, USA), may be used. The use of this Fc-fusion technology allows the creation of longer-acting biopharmaceuticals and may, for example, consist of two copies of the mutein linked to the Fc region of an antibody to improve pharmacokinetics, solubility, and production efficiency.

Examples of albumin binding peptides that can be used to extend the serum half-life of a lipocalin mutein are, for instance, those having a Cys-Xaa₁-Xaa₂-Xaa₃-Xaa₄-Cys consensus sequence, wherein Xaa₁ is Asp, Asn, Ser, Thr, or Trp; Xaa₂ is Asn, Gln, His, Ile, Leu, or Lys; Xaa₃ is Ala, Asp, Phe, Trp, or Tyr; and Xaa₄ is Asp, Gly, Leu, Phe, Ser, or Thr as described in U.S. Patent Publication No. 2003/0069395 or Dennis et al. (2002) J. Biol. Chem., 277(38):35035-43. The albumin binding protein fused or conjugated to a lipocalin mutein to extend serum half-life may be a bacterial albumin binding protein, an antibody, an antibody fragment including domain antibodies (see U.S. Pat. No. 6,696,245, for example), or a lipocalin mutein with binding activity for albumin. Examples of bacterial albumin binding proteins include streptococcal protein G (König and Skerra, J Immunol Methods, 1998, 218(1-2):73-83).

If the albumin-binding protein is an antibody fragment it may be a domain antibody. Domain Antibodies (dAbs) are engineered to allow precise control over biophysical properties and in vivo half-life to create the optimal safety and efficacy product profile. Domain Antibodies are for example commercially available from Domantis Ltd. (Cambridge, UK, and MA, USA).

Particularly, albumin itself (Osborn et al., J Pharmacol Exp Ther, 2002, 303(2):540-8), or a biologically active fragment of albumin can be used as a partner of a lipocalin mutein of the disclosure to extend serum half-life. The term “albumin” includes all mammal albumins such as human serum albumin or bovine serum albumin or rat albumin. The albumin or fragment thereof can be recombinantly produced as described in U.S. Pat. No. 5,728,553 or European Patent Publication Nos. EP 0 330 451 and EP 0 361 991. Accordingly, recombinant human albumin (e.g., Recombumin® from Novozymes Delta Ltd., Nottingham, UK) can be conjugated or fused to a lipocalin mutein of the disclosure.

If a transferrin is used as a partner to extend the serum half-life of the lipocalin muteins of the disclosure, the muteins can be genetically fused to the N- or C-terminus, or both, of non-glycosylated transferrin. Non-glycosylated transferrin has a half-life of 14-17 days, and a transferrin fusion protein will similarly have an extended half-life. The transferrin carrier also provides high bioavailability, biodistribution and circulating stability. This technology is commercially available from BioRexis (BioRexis Pharmaceutical Corporation, PA, USA). Recombinant human transferrin (DeltaFerrin™) for use as a protein stabilizer/half-life extension partner is also commercially available from Novozymes Delta Ltd. (Nottingham, UK).

Yet another alternative to prolong the half-life of the lipocalin muteins of the disclosure is to fuse to the N- or C-terminus of a mutein a long, unstructured, flexible glycine-rich sequence (for example poly-glycine with about 20 to 80 consecutive glycine residues). This approach disclosed in International Patent Publication No. WO 2007/038619, for example, has also been term “rPEG” (recombinant PEG).

In some further embodiments, a lipocalin mutein disclosed herein may be fused or conjugated, at its N-terminus and/or its C-terminus, to a moiety that may confer new characteristics to the lipocalin muteins of the disclosure such as enzymatic activity or binding affinity for other targets. Examples of suitable fusion partners are alkaline phosphatase, horseradish peroxidase, glutathione S-transferase, the albumin-binding domain of protein G, protein A, antibodies or antibody fragments, oligomerization domains, other lipocalin muteins, or toxins.

In particular, it may be possible to fuse a lipocalin mutein disclosed herein with a separate enzyme active site such that both “subunits” of the resulting fusion protein act together on a given therapeutic target. The binding domain of the lipocalin mutein attaches to the disease-causing target, allowing the enzyme domain to abolish the biological function of the target.

It is also possible to fuse a lipocalin mutein disclosed herein with a second “subunit,” which is an antibody, an antibody active fragment, or another lipocalin mutein, such that the resulting fusion protein acts on both the target of the lipocalin mutein and one other given therapeutic target.

A lipocalin mutein that binds a given non-natural target may be fused to another lipocalin mutein that binds to the same non-natural target. Such fusion may lead to a stronger binding and thus to an increased potency as a result of avidity. The increase in size of this fusion may lead to a longer exposure in plasma and/or retention in tissue (e.g., lung tissue) as compared to the single lipocalin muteins. Both lipocalin muteins may bind to different epitopes on the same target in order to reach a broader coverage of the non-natural target, e.g., CTGF, and thereby the blockade of more potential interaction partners of the non-natural target, e.g., CTGF interaction partners. Preferably, such epitopes are non-overlapping. If the two lipocalin muteins bind to different epitopes, it is also preferred that the two lipocalin muteins do not compete with each other for target binding. Binding competition can, e.g., be determined as essentially described in Example 8. Thus, the present disclosure also relates to a fusion protein comprising two lipocalin muteins binding to different (e.g., non-overlapping) epitopes of the same non-natural target (e.g., a target protein, such as CTGF).

A lipocalin mutein of the disclosure can be fused to another lipocalin mutein of the disclosure. The present disclosure thus also relates to a fusion protein comprising two lipocalin muteins of the disclosure. Both lipocalin muteins may comprise the same amino acid sequence. However, it is preferred that the two lipocalin muteins comprise different amino acid sequences. Both lipocalin muteins may bind to the same epitope on CTGF. However, it is preferred that both lipocalins bind to different epitopes. Thus, both lipocalins may belong to different groups selected from the group consisting of N group, NP group, and C group. As an illustrative example, a lipocalin mutein of the disclosure that belongs to the N group may be fused to a lipocalin mutein of the disclosure that belongs to the NP group, a lipocalin mutein of the disclosure that belongs to the C group may be fused to a lipocalin mutein of the disclosure that belongs to the N group, and/or a lipocalin mutein of the disclosure that belongs to the NP group may be fused to a lipocalin mutein of the disclosure that belongs to the N group, with the latter being preferred. A fusion protein may comprise a linker as disclosed herein. The linker may connect the two lipocalin muteins with each other.

In some embodiments, two lipocalin muteins of the disclosure that are comprised in a fusion protein may comprise the following sets of mutated amino acid residues in comparison with the linear polypeptide sequence of mature hNGAL (SEQ ID NO: 1), respectively:
(a) Leu 36→Arg, Ala 40→Asn, Ile 41→Gln, Asp 47→Ser, Gln 49→Pro, Tyr 52→Trp, Ser 68→His, Leu 70→His, Arg 72→Ser, Lys 73→Thr, Asp 77→Arg, Trp 79→Ile, Arg 81→Asp, Leu 94→Ala, Asn 96→Ala, Tyr 100→Gly, Tyr 106→Pro, Lys 125→Trp, Ser 127→Thr, Tyr 132→Trp, and Lys 134→Thr; and
   Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp;
(b) Leu 36→Arg, Ala 40→Asn, Ile 41→Gln, Asp 47→Ser, Gln 49→Pro, Tyr 52→Trp, Ser 68→His, Leu 70→His, Arg 72→Ser, Lys 73→Thr, Asp 77→Arg, Trp 79→Ile, Arg 81→Asp, Leu 94→Ala, Asn 96→Ala, Tyr 100→Gly, Tyr 106→Pro, Lys 125→Trp, Ser 127→Thr, Tyr 132→Trp, and Lys 134→Thr; and
   Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val;
(c) Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 106→Gln, Phe 123→Val, Ser 127→Trp, Gln 128→Pro, Arg 130→Leu, and Lys 134→Gln, Thr 136→Val; and
   Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val;
(d) Leu 36→Arg, Ala 40→Asn, Ile 41→Arg, Gln 49→Pro, Tyr 52→Trp, Ser 68→His, Leu 70→His, Arg 72→Met, Lys 73→Thr, Asp 77→Arg, Trp 79→Ile, Arg 81→Asp, Asn 96→Ala, Tyr 100→Gly, Leu 103→Met, Tyr 106→Pro, Val 110→Ile, Lys 125→Trp, Ser 127→Asn, Tyr 132→Trp, and Lys 134→Thr; and
   Ala 40→Lys, Glu 44→Ile, Asp 47→Gln, Gln 49→Ala, Tyr 52→Phe, Leu 70→Arg, Arg 72→Glu, Lys 73→Ala, Lys 74→Arg, Lys 75→Ser, Asp 77→Ser, Trp 79→Thr, Arg 81→Lys, Leu 94→Ala, Tyr 100→Ala, Leu 103→Gln, Tyr 106→Thr, Lys 125→His, Tyr 132→Thr, and Lys 134→Val;
(e) Ala 40→Lys, Glu 44→Ile, Asp 47→Gln, Gln 49→Ala, Tyr 52→Phe, Leu 70→Arg, Arg 72→Glu, Lys 73→Ala, Lys 74→Arg, Lys 75→Ser, Asp 77→Ser, Trp 79→Thr, Arg 81→Lys, Leu 94→Ala, Tyr 100→Ala, Leu 103→Gln, Tyr 106→Thr, Lys 125→His, Tyr 132→Thr, and Lys 134→Val; and
   Leu 36→Arg, Ala 40→Asn, Ile 41→Arg, Gln 49→Pro, Tyr 52→Trp, Ser 68→His, Leu 70→His, Arg 72→Met, Lys 73→Thr, Asp 77→Arg, Trp 79→Ile, Arg 81→Asp, Asn 96→Ala, Tyr 100→Gly, Leu 103→Met, Tyr 106→Pro, Val 110→Ile, Lys 125→Trp, Ser 127→Asn, Tyr 132→Trp, and Lys 134→Thr;
(f) Ala 40→Lys, Glu 44→Ile, Asp 47→Gln, Gln 49→Ala, Tyr 52→Phe, Leu 70→Arg, Arg 72→Glu, Lys 73→Ala, Lys 74→Arg, Lys 75→Ser, Asp 77→Ser, Trp 79→Thr, Arg 81→Lys, Leu 94→Ala, Tyr 100→Ala, Leu 103→Gln, Tyr 106→Thr, Lys 125→His, Tyr 132→Thr, and Lys 134→Val; and
   Leu 36→Trp, Ala 40→Ile, Ile 41→Lys, Gln 49→Phe, Tyr 52→Ser, Ser 68→Glu, Leu 70→Val, Arg 72→Glu, Lys 73→Gln, Asp 77→His, Trp 79→Ile, Arg 81→Lys, Asn 96→Asp, Tyr 100→Pro, Leu 103→Glu, Tyr 106→Ser, Ser 127→Phe, Tyr 132→Phe, and Lys 134→Trp;
(g) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Ile 80→Ser, Arg 81→Lys, Leu 94→Ala, Tyr 100→Ala, Leu 103→Gln, Tyr 106→Thr, Lys 125→His, Tyr 132→Thr, and Lys 134→Val; and
   Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp;
(h) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Ile 80→Ser, Arg 81→Lys, Leu 94→Ala, Tyr 100→Ala, Leu 103→Gln, Tyr 106→Thr, Lys 125→His, Tyr 132→Thr, and Lys 134→Val; and
   Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 106→Gln, Phe 123→Val, Ser 127→Trp, Gln 128→Pro, Arg 130→Leu, and Lys 134→Gln, Thr 136→Val;
(i) Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp; and
   Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val;
(j) Asp 47→Tyr, Gln 49→Ala, Tyr 52→Phe, Leu 70→Arg, Arg 72→Glu, Lys 73→Asn, Lys 75→Ser, Asp 77→Val, Trp 79→Thr, Arg 81→Lys, Leu 94→Thr, Tyr 100→Ala, Leu 103→Gln, Tyr 106→Thr, Lys 125→His, Tyr 132→Phe, and Lys 134→Val; and
   Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp;
(k) Asp 47→Tyr, Gln 49→Ala, Tyr 52→Phe, Leu 70→Arg, Arg 72→Glu, Lys 73→Asn, Lys 75→Ser, Asp 77→Val, Trp 79→Thr, Arg 81→Lys, Leu 94→Thr, Tyr 100→Ala, Leu 103→Gln, Tyr 106→Thr, Lys 125→His, Tyr 132→Phe, and Lys 134→Val; and
   Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 106→Gln, Phe 123→Val, Ser 127→Trp, Gln 128→Pro, Arg 130→Leu, and Lys 134→Gln, Thr 136→Val;
(l) Asp 47→Tyr, Gln 49→Ala, Tyr 52→Phe, Leu 70→Arg, Arg 72→Glu, Lys 73→Asn, Lys 75→Ser, Asp 77→Val, Trp 79→Thr, Ile 80→Ser, Arg 81→Lys, Leu 94→Thr, Tyr 100→Ala, Leu 103→Gln, Tyr 106→Thr, Lys 125→His, Tyr 132→Phe, and Lys 134→Val; and
   Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp;
(m) Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val; and
   Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp;
(n) Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val; and
   Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 106→Gln, Phe 123→Val, Ser 127→Trp, Gln 128→Pro, Arg 130→Leu, and Lys 134→Gln, Thr 136→Val; or
(o) Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val; and
   Leu 36→Trp, Ala 40→Val, Ile 41→Lys, Asp 47→Gln, Gln 49→Leu, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp.

In some embodiments, two lipocalin muteins of the disclosure that are comprised in a fusion protein may comprise the following sets of mutated amino acid residues in comparison with the linear polypeptide sequence of mature hNGAL (SEQ ID NO: 1), respectively:
(a) Leu 36→Arg, Ala 40→Asn, Ile 41→Gln, Asp 47→Ser, Gln 49→Pro, Tyr 52→Trp, Asn 65→Asp, Ser 68→His, Leu 70→His, Arg 72→Ser, Lys 73→Thr, Asp 77→Arg, Trp 79→Ile, Arg 81→Asp, Cys 87→Ser, Leu 94→Ala, Asn 96→Ala, Tyr 100→Gly, Tyr 106→Pro, Lys 125→Trp, Ser 127→Thr, Tyr 132→Trp, and Lys 134→Thr; and
   Leu 36→Trp, Ala 40→Val, Ile 41→Lys, Asp 47→Gln, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Leu 94→Ala, Asn 96→Asp, Tyr 100→Pro, Leu 103→Tyr, Tyr 106→Ser, Tyr 132→Phe, and Lys 134→Trp;
(b) Leu 36→Arg, Ala 40→Asn, Ile 41→Gln, Asp 47→Ser, Gln 49→Pro, Tyr 52→Trp, Asn 65→Asp, Ser 68→His, Leu 70→His, Arg 72→Ser, Lys 73→Thr, Asp 77→Arg, Trp 79→Ile, Arg 81→Asp, Cys 87→Ser, Leu 94→Ala, Asn 96→Ala, Tyr 100→Gly, Tyr 106→Pro, Lys 125→Trp, Ser 127→Thr, Tyr 132→Trp, and Lys 134→Thr; and
   Asp 47→Gln, Gln 49→Ala, Tyr 52→Phe, Asn 65→Asp, Leu 70→Arg, Arg 72→Glu, Lys 73→Ala, Lys 75→Ser, Asp 77→Lys, Trp 79→Thr, Arg 81→Lys, Cys 87→Ser, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val;
(c) Ala 40→Tyr, Ile 41→Arg, Gln 49→Ser, Tyr 52→Gly, Asn 65→Asp, Ser 68→Gln, Leu 70→Gln, Arg 72→Asp, Lys 73→Asp, Asp 77→Leu, Trp 79→Val, Arg 81→Glu, Cys 87→Ser, Asn 96→Ala, Tyr 106→Gln, Phe 123→Val, Ser 127→Trp, Gln 128→Pro, Arg 130→Leu, and Lys 134→Gln, Thr 136→Val; and
   Asp 47→Gln, Gln 49→Ala, Tyr 52→Phe, Asn 65→Asp, Leu 70→Arg, Arg 72→Glu, Lys 73→Ala, Lys 75→Ser, Asp 77→Lys, Trp 79→Thr, Arg 81→Lys, Cys 87→Ser, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val;
(d) Gln 28→His, Leu 36→Arg, Ala 40→Asn, Ile 41→Arg, Gln 49→Pro, Tyr 52→Trp, Asn 65→Asp, Ser 68→His, Leu 70→His, Arg 72→Met, Lys 73→Thr, Asp 77→Arg, Trp 79→Ile, Arg 81→Asp, Cys 87→Ser, Asn 96→Ala, Tyr 100→Gly, Leu 103→Met, Tyr 106→Pro, Val 110→Ile, Lys 125→Trp, Ser 127→Asn, Tyr 132→Trp, and Lys 134→Thr; and
   Gln 28→His, Ala 40→Lys, Glu 44→Ile, Asp 47→Gln, Gln 49→Ala, Tyr 52→Phe, Asn 65→Asp, Leu 70→Arg, Arg 72→Glu, Lys 73→Ala, Lys 74→Arg, Lys 75→Ser, Asp 77→Ser, Trp 79→Thr, Arg 81→Lys, Cys 87→Ser, Leu 94→Ala, Tyr 100→Ala, Leu 103→Gln, Tyr 106→Thr, Lys 125→His, Tyr 132→Thr, and Lys 134→Val;
(e) Gln 28→His, Ala 40→Lys, Glu 44→Ile, Asp 47→Gln, Gln 49→Ala, Tyr 52→Phe, Asn 65→Asp, Leu 70→Arg, Arg 72→Glu, Lys 73→Ala, Lys 74→Arg, Lys 75→Ser, Asp 77→Ser, Trp 79→Thr, Arg 81→Lys, Cys 87→Ser, Leu 94→Ala, Tyr 100→Ala, Leu 103→Gln, Tyr 106→Thr, Lys 125→His, Tyr 132→Thr, and Lys 134→Val; and
   Gln 28→His, Leu 36→Arg, Ala 40→Asn, Ile 41→Arg, Gln 49→Pro, Tyr 52→Trp, Asn 65→Asp, Ser 68→His, Leu 70→His, Arg 72→Met, Lys 73→Thr, Asp 77→Arg, Trp 79→Ile, Arg 81→Asp, Cys 87→Ser, Asn 96→Ala, Tyr 100→Gly, Leu 103→Met, Tyr 106→Pro, Val 110→Ile, Lys 125→Trp, Ser 127→Asn, Tyr 132→Trp, and Lys 134→Thr;
(f) Gln 28→His, Ala 40→Lys, Glu 44→Ile, Asp 47→Gln, Gln 49→Ala, Tyr 52→Phe, Asn 65→Asp, Leu 70→Arg, Arg 72→Glu, Lys 73→Ala, Lys 74→Arg, Lys 75→Ser, Asp 77→Ser, Trp 79→Thr, Arg 81→Lys, Cys 87→Ser, Leu 94→Ala, Tyr 100→Ala, Leu 103→Gln, Tyr 106→Thr, Lys 125→His, Tyr 132→Thr, and Lys 134→Val; and
   Gln 28→His, Leu 36→Trp, Ala 40→Ile, Ile 41→Lys, Gln 49→Phe, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Asn 96→Asp, Tyr 100→Pro, Leu 103→Glu, Tyr 106→Ser, Ser 127→Phe, Tyr 132→Phe, and Lys 134→Trp;
(g) Asp 47→Gln, Gln 49→Ala, Tyr 52→Phe, Asn 65→Asp, Leu 70→Arg, Arg 72→Glu, Lys 73→Ala, Lys 75→Ser, Asp 77→Ser, Trp 79→Thr, Ile 80→Ser, Arg 81→Lys, Cys 87→Ser, Leu 94→Ala, Tyr 100→Ala, Leu 103→Gln, Tyr 106→Thr, Lys 125→His, Tyr 132→Thr, and Lys 134→Val; and
   Leu 36→Trp, Ala 40→Val, Ile 41→Lys, Asp 47→Gln, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Leu 94→Ala, Asn 96→Asp, Tyr 100→Pro, Leu 103→Tyr, Tyr 106→Ser, Tyr 132→Phe, and Lys 134→Trp;
(h) Asp 47→Gln, Gln 49→Ala, Tyr 52→Phe, Asn 65→Asp, Leu 70→Arg, Arg 72→Glu, Lys 73→Ala, Lys 75→Ser, Asp 77→Ser, Trp 79→Thr, Ile 80→Ser, Arg 81→Lys, Cys 87→Ser, Leu 94→Ala, Tyr 100→Ala, Leu 103→Gln, Tyr 106→Thr, Lys 125→His, Tyr 132→Thr, and Lys 134→Val; and
   Ala 40→Tyr, Ile 41→Arg, Gln 49→Ser, Tyr 52→Gly, Asn 65→Asp, Ser 68→Gln, Leu 70→Gln, Arg 72→Asp, Lys 73→Asp, Asp 77→Leu, Trp 79→Val, Arg 81→Glu, Cys 87→Ser, Asn 96→Ala, Tyr 106→Gln, Phe 123→Val, Ser 127→Trp, Gln 128→Pro, Arg 130→Leu, and Lys 134→Gln, Thr 136→Val;
(i) Leu 36→Trp, Ala 40→Val, Ile 41→Lys, Asp 47→Gln, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Leu 94→Ala, Asn 96→Asp, Tyr 100→Pro, Leu 103→Tyr, Tyr 106→Ser, Tyr 132→Phe, and Lys 134→Trp; and
   Asp 47→Gln, Gln 49→Ala, Tyr 52→Phe, Asn 65→Asp, Leu 70→Arg, Arg 72→Glu, Lys 73→Ala, Lys 75→Ser, Asp 77→Lys, Trp 79→Thr, Arg 81→Lys, Cys 87→Ser, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val;
(j) Asp 47→Tyr, Gln 49→Ala, Tyr 52→Phe, Asn 65→Asp, Leu 70→Arg, Arg 72→Glu, Lys 73→Asn, Lys 75→Ser, Asp 77→Val, Trp 79→Thr, Arg 81→Lys, Cys 87→Ser, Leu 94→Thr, Tyr 100→Ala, Leu 103→Gln, Tyr 106→Thr, Lys 125→His, Tyr 132→Phe, and Lys 134→Val; and
   Leu 36→Trp, Ala 40→Val, Ile 41→Lys, Asp 47→Gln, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Leu 94→Ala, Asn 96→Asp, Tyr 100→Pro, Leu 103→Tyr, Tyr 106→Ser, Tyr 132→Phe, and Lys 134→Trp;
(k) Asp 47→Tyr, Gln 49→Ala, Tyr 52→Phe, Asn 65→Asp, Leu 70→Arg, Arg 72→Glu, Lys 73→Asn, Lys 75→Ser, Asp 77→Val, Trp 79→Thr, Arg 81→Lys, Cys 87→Ser, Leu 94→Thr, Tyr 100→Ala, Leu 103→Gln, Tyr 106→Thr, Lys 125→His, Tyr 132→Phe, and Lys 134→Val; and
   Ala 40→Tyr, Ile 41→Arg, Gln 49→Ser, Tyr 52→Gly, Asn 65→Asp, Ser 68→Gln, Leu 70→Gln, Arg 72→Asp, Lys 73→Asp, Asp 77→Leu, Trp 79→Val, Arg 81→Glu, Cys 87→Ser, Asn 96→Ala, Tyr 106→Gln, Phe 123→Val, Ser 127→Trp, Gln 128→Pro, Arg 130→Leu, and Lys 134→Gln, Thr 136→Val;
(l) Asp 47→Tyr, Gln 49→Ala, Tyr 52→Phe, Asn 65→Asp, Leu 70→Arg, Arg 72→Glu, Lys 73→Asn, Lys 75→Ser, Asp 77→Val, Trp 79→Thr, Ile 80→Ser, Arg 81→Lys, Cys 87→Ser, Leu 94→Thr, Tyr 100→Ala, Leu 103→Gln, Tyr 106→Thr, Lys 125→His, Tyr 132→Phe, and Lys 134→Val; and
   Leu 36→Trp, Ala 40→Val, Ile 41→Lys, Asp 47→Gln, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Leu 94→Ala, Asn 96→Asp, Tyr 100→Pro, Leu 103→Tyr, Tyr 106→Ser, Tyr 132→Phe, and Lys 134→Trp;
(m) Asp 47→Gln, Gln 49→Ala, Tyr 52→Phe, Asn 65→Asp, Leu 70→Arg, Arg 72→Glu, Lys 73→Ala, Lys 75→Ser, Asp 77→Lys, Trp 79→Thr, Arg 81→Lys, Cys 87→Ser, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val; and
   Leu 36→Trp, Ala 40→Val, Ile 41→Lys, Asp 47→Gln, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Leu 94→Ala, Asn 96→Asp, Tyr 100→Pro, Leu 103→Tyr, Tyr 106→Ser, Tyr 132→Phe, and Lys 134→Trp;
(n) Asp 47→Gln, Gln 49→Ala, Tyr 52→Phe, Asn 65→Asp, Leu 70→Arg, Arg 72→Glu, Lys 73→Ala, Lys 75→Ser, Asp 77→Lys, Trp 79→Thr, Arg 81→Lys, Cys 87→Ser, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val; and
   Ala 40→Tyr, Ile 41→Arg, Gln 49→Ser, Tyr 52→Gly, Asn 65→Asp, Ser 68→Gln, Leu 70→Gln, Arg 72→Asp, Lys 73→Asp, Asp 77→Leu, Trp 79→Val, Arg 81→Glu, Cys 87→Ser, Asn 96→Ala, Tyr 106→Gln, Phe 123→Val, Ser 127→Trp, Gln 128→Pro, Arg 130→Leu, and Lys 134→Gln, Thr 136→Val; or
(o) Asp 47→Gln, Gln 49→Ala, Tyr 52→Phe, Asn 65→Asp, Leu 70→Arg, Arg 72→Glu, Lys 73→Ala, Lys 75→Ser, Asp 77→Lys, Trp 79→Thr, Arg 81→Lys, Cys 87→Ser, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val; and
   Leu 36→Trp, Ala 40→Val, Ile 41→Lys, Asp 47→Gln, Gln 49→Leu, Tyr 52→Ser, Asn 65→Asp, Ser 68→Glu, Leu 70→Val, Arg 72→Glu, Lys 73→Gln, Asp 77→His, Trp 79→Ile, Arg 81→Lys, Cys 87→Ser, Leu 94→Ala, Asn 96→Asp, Tyr 100→Pro, Leu 103→Tyr, Tyr 106→Ser, Tyr 132→Phe, and Lys 134→Trp.

In some embodiments, two lipocalin muteins of the disclosure that are comprised in a fusion protein may comprise the following amino acid sequences, respectively:
(a) an amino acid sequence having at least 80% identity to the amino acid sequence shown in SEQ ID NO: 6 and an amino acid sequence having at least 80% identity to the amino acid sequence shown in SEQ ID NO: 31;
(b) an amino acid sequence having at least 80% identity to the amino acid sequence shown in SEQ ID NO: 6 and an amino acid sequence having at least 80% identity to the amino acid sequence shown in SEQ ID NO: 15;
(c) an amino acid sequence having at least 80% identity to the amino acid sequence shown in SEQ ID NO: 28 and an amino acid sequence having at least 80% identity to the amino acid sequence shown in SEQ ID NO: 15;
(d) an amino acid sequence having at least 80% identity to the amino acid sequence shown in SEQ ID NO: 4 and an amino acid sequence having at least 80% identity to the amino acid sequence shown in SEQ ID NO: 9;
(e) an amino acid sequence having at least 80% identity to the amino acid sequence shown in SEQ ID NO: 9 and an amino acid sequence having at least 80% identity to the amino acid sequence shown in SEQ ID NO: 4;
(f) an amino acid sequence having at least 80% identity to the amino acid sequence shown in SEQ ID NO: 9 and an amino acid sequence having at least 80% identity to the amino acid sequence shown in SEQ ID NO: 30;
(g) an amino acid sequence having at least 80% identity to the amino acid sequence shown in SEQ ID NO: 11 and an amino acid sequence having at least 80% identity to the amino acid sequence shown in SEQ ID NO: 31;
(h) an amino acid sequence having at least 80% identity to the amino acid sequence shown in SEQ ID NO: 11 and an amino acid sequence having at least 80% identity to the amino acid sequence shown in SEQ ID NO: 28;
(i) an amino acid sequence having at least 80% identity to the amino acid sequence shown in SEQ ID NO: 31 and an amino acid sequence having at least 80% identity to the amino acid sequence shown in SEQ ID NO: 15;
(j) an amino acid sequence having at least 80% identity to the amino acid sequence shown in SEQ ID NO: 12 and an amino acid sequence having at least 80% identity to the amino acid sequence shown in SEQ ID NO: 31;
(k) an amino acid sequence having at least 80% identity to the amino acid sequence shown in SEQ ID NO: 12 and an amino acid sequence having at least 80% identity to the amino acid sequence shown in SEQ ID NO: 28;
(l) an amino acid sequence having at least 80% identity to the amino acid sequence shown in SEQ ID NO: 13 and an amino acid sequence having at least 80% identity to the amino acid sequence shown in SEQ ID NO: 31;
(m) an amino acid sequence having at least 80% identity to the amino acid sequence shown in SEQ ID NO: 15 and an amino acid sequence having at least 80% identity to the amino acid sequence shown in SEQ ID NO: 31;
(n) an amino acid sequence having at least 80% identity to the amino acid sequence shown in SEQ ID NO: 15 and an amino acid sequence having at least 80% identity to the amino acid sequence shown in SEQ ID NO: 28; or
(o) an amino acid sequence having at least 80% identity to the amino acid sequence shown in SEQ ID NO: 15 and an amino acid sequence having at least 80% identity to the amino acid sequence shown in SEQ ID NO: 35.

In some embodiments, two lipocalin muteins of the disclosure that are comprised in a fusion protein may comprise the following amino acid sequences, respectively:
(a) an amino acid sequence having at least 85% identity to the amino acid sequence shown in SEQ ID NO: 6 and an amino acid sequence having at least 85% identity to the amino acid sequence shown in SEQ ID NO: 31;
(b) an amino acid sequence having at least 85% identity to the amino acid sequence shown in SEQ ID NO: 6 and an amino acid sequence having at least 85% identity to the amino acid sequence shown in SEQ ID NO: 15;
(c) an amino acid sequence having at least 85% identity to the amino acid sequence shown in SEQ ID NO: 28 and an amino acid sequence having at least 85% identity to the amino acid sequence shown in SEQ ID NO: 15;
(d) an amino acid sequence having at least 85% identity to the amino acid sequence shown in SEQ ID NO: 4 and an amino acid sequence having at least 85% identity to the amino acid sequence shown in SEQ ID NO: 9;
(e) an amino acid sequence having at least 85% identity to the amino acid sequence shown in SEQ ID NO: 9 and an amino acid sequence having at least 85% identity to the amino acid sequence shown in SEQ ID NO: 4;
(f) an amino acid sequence having at least 85% identity to the amino acid sequence shown in SEQ ID NO: 9 and an amino acid sequence having at least 85% identity to the amino acid sequence shown in SEQ ID NO: 30;
(g) an amino acid sequence having at least 85% identity to the amino acid sequence shown in SEQ ID NO: 11 and an amino acid sequence having at least 85% identity to the amino acid sequence shown in SEQ ID NO: 31;
(h) an amino acid sequence having at least 85% identity to the amino acid sequence shown in SEQ ID NO: 11 and an amino acid sequence having at least 85% identity to the amino acid sequence shown in SEQ ID NO: 28;
(i) an amino acid sequence having at least 85% identity to the amino acid sequence shown in SEQ ID NO: 31 and an amino acid sequence having at least 85% identity to the amino acid sequence shown in SEQ ID NO: 15;
(j) an amino acid sequence having at least 85% identity to the amino acid sequence shown in SEQ ID NO: 12 and an amino acid sequence having at least 85% identity to the amino acid sequence shown in SEQ ID NO: 31;
(k) an amino acid sequence having at least 85% identity to the amino acid sequence shown in SEQ ID NO: 12 and an amino acid sequence having at least 85% identity to the amino acid sequence shown in SEQ ID NO: 28;
(l) an amino acid sequence having at least 85% identity to the amino acid sequence shown in SEQ ID NO: 13 and an amino acid sequence having at least 85% identity to the amino acid sequence shown in SEQ ID NO: 31;
(m) an amino acid sequence having at least 85% identity to the amino acid sequence shown in SEQ ID NO: 15 and an amino acid sequence having at least 85% identity to the amino acid sequence shown in SEQ ID NO: 31;
(n) an amino acid sequence having at least 85% identity to the amino acid sequence shown in SEQ ID NO: 15 and an amino acid sequence having at least 85% identity to the amino acid sequence shown in SEQ ID NO: 28; or
(o) an amino acid sequence having at least 85% identity to the amino acid sequence shown in SEQ ID NO: 15 and an amino acid sequence having at least 85% identity to the amino acid sequence shown in SEQ ID NO: 35.

In some embodiments, two lipocalin muteins of the disclosure that are comprised in a fusion protein may comprise the following amino acid sequences, respectively:
(a) an amino acid sequence having at least 90% identity to the amino acid sequence shown in SEQ ID NO: 6 and an amino acid sequence having at least 90% identity to the amino acid sequence shown in SEQ ID NO: 31;
(b) an amino acid sequence having at least 90% identity to the amino acid sequence shown in SEQ ID NO: 6 and an amino acid sequence having at least 90% identity to the amino acid sequence shown in SEQ ID NO: 15;
(c) an amino acid sequence having at least 90% identity to the amino acid sequence shown in SEQ ID NO: 28 and an amino acid sequence having at least 90% identity to the amino acid sequence shown in SEQ ID NO: 15;
(d) an amino acid sequence having at least 90% identity to the amino acid sequence shown in SEQ ID NO: 4 and an amino acid sequence having at least 90% identity to the amino acid sequence shown in SEQ ID NO: 9;
(e) an amino acid sequence having at least 90% identity to the amino acid sequence shown in SEQ ID NO: 9 and an amino acid sequence having at least 90% identity to the amino acid sequence shown in SEQ ID NO: 4;
(f) an amino acid sequence having at least 90% identity to the amino acid sequence shown in SEQ ID NO: 9 and an amino acid sequence having at least 90% identity to the amino acid sequence shown in SEQ ID NO: 30;
(g) an amino acid sequence having at least 90% identity to the amino acid sequence shown in SEQ ID NO: 11 and an amino acid sequence having at least 90% identity to the amino acid sequence shown in SEQ ID NO: 31;
(h) an amino acid sequence having at least 90% identity to the amino acid sequence shown in SEQ ID NO: 11 and an amino acid sequence having at least 90% identity to the amino acid sequence shown in SEQ ID NO: 28;
(i) an amino acid sequence having at least 90% identity to the amino acid sequence shown in SEQ ID NO: 31 and an amino acid sequence having at least 90% identity to the amino acid sequence shown in SEQ ID NO: 15;
(j) an amino acid sequence having at least 90% identity to the amino acid sequence shown in SEQ ID NO: 12 and an amino acid sequence having at least 90% identity to the amino acid sequence shown in SEQ ID NO: 31;
(k) an amino acid sequence having at least 90% identity to the amino acid sequence shown in SEQ ID NO: 12 and an amino acid sequence having at least 90% identity to the amino acid sequence shown in SEQ ID NO: 28;
(l) an amino acid sequence having at least 90% identity to the amino acid sequence shown in SEQ ID NO: 13 and an amino acid sequence having at least 90% identity to the amino acid sequence shown in SEQ ID NO: 31;
(m) an amino acid sequence having at least 90% identity to the amino acid sequence shown in SEQ ID NO: 15 and an amino acid sequence having at least 90% identity to the amino acid sequence shown in SEQ ID NO: 31;
(n) an amino acid sequence having at least 90% identity to the amino acid sequence shown in SEQ ID NO: 15 and an amino acid sequence having at least 90% identity to the amino acid sequence shown in SEQ ID NO: 28; or
(o) an amino acid sequence having at least 90% identity to the amino acid sequence shown in SEQ ID NO: 15 and an amino acid sequence having at least 90% identity to the amino acid sequence shown in SEQ ID NO: 35.

In some embodiments, two lipocalin muteins of the disclosure that are comprised in a fusion protein may comprise the following amino acid sequences, respectively:
(a) an amino acid sequence having at least 95% identity to the amino acid sequence shown in SEQ ID NO: 6 and an amino acid sequence having at least 95% identity to the amino acid sequence shown in SEQ ID NO: 31;
(b) an amino acid sequence having at least 95% identity to the amino acid sequence shown in SEQ ID NO: 6 and an amino acid sequence having at least 95% identity to the amino acid sequence shown in SEQ ID NO: 15;
(c) an amino acid sequence having at least 95% identity to the amino acid sequence shown in SEQ ID NO: 28 and an amino acid sequence having at least 95% identity to the amino acid sequence shown in SEQ ID NO: 15;
(d) an amino acid sequence having at least 95% identity to the amino acid sequence shown in SEQ ID NO: 4 and an amino acid sequence having at least 95% identity to the amino acid sequence shown in SEQ ID NO: 9;
(e) an amino acid sequence having at least 95% identity to the amino acid sequence shown in SEQ ID NO: 9 and an amino acid sequence having at least 95% identity to the amino acid sequence shown in SEQ ID NO: 4;
(f) an amino acid sequence having at least 95% identity to the amino acid sequence shown in SEQ ID NO: 9 and an amino acid sequence having at least 95% identity to the amino acid sequence shown in SEQ ID NO: 30;
(g) an amino acid sequence having at least 95% identity to the amino acid sequence shown in SEQ ID NO: 11 and an amino acid sequence having at least 95% identity to the amino acid sequence shown in SEQ ID NO: 31;
(h) an amino acid sequence having at least 95% identity to the amino acid sequence shown in SEQ ID NO: 11 and an amino acid sequence having at least 95% identity to the amino acid sequence shown in SEQ ID NO: 28;
(i) an amino acid sequence having at least 95% identity to the amino acid sequence shown in SEQ ID NO: 31 and an amino acid sequence having at least 95% identity to the amino acid sequence shown in SEQ ID NO: 15;
(j) an amino acid sequence having at least 95% identity to the amino acid sequence shown in SEQ ID NO: 12 and an amino acid sequence having at least 95% identity to the amino acid sequence shown in SEQ ID NO: 31;
(k) an amino acid sequence having at least 95% identity to the amino acid sequence shown in SEQ ID NO: 12 and an amino acid sequence having at least 95% identity to the amino acid sequence shown in SEQ ID NO: 28;
(l) an amino acid sequence having at least 95% identity to the amino acid sequence shown in SEQ ID NO: 13 and an amino acid sequence having at least 95% identity to the amino acid sequence shown in SEQ ID NO: 31;
(m) an amino acid sequence having at least 95% identity to the amino acid sequence shown in SEQ ID NO: 15 and an amino acid sequence having at least 95% identity to the amino acid sequence shown in SEQ ID NO: 31;
(n) an amino acid sequence having at least 95% identity to the amino acid sequence shown in SEQ ID NO: 15 and an amino acid sequence having at least 95% identity to the amino acid sequence shown in SEQ ID NO: 28; or
(o) an amino acid sequence having at least 95% identity to the amino acid sequence shown in SEQ ID NO: 15 and an amino acid sequence having at least 95% identity to the amino acid sequence shown in SEQ ID NO: 35.

In some embodiments, two lipocalin muteins of the disclosure that are comprised in a fusion protein may comprise the following amino acid sequences, respectively:
(a) an amino acid sequence having at least 98% identity to the amino acid sequence shown in SEQ ID NO: 6 and an amino acid sequence having at least 98% identity to the amino acid sequence shown in SEQ ID NO: 31;
(b) an amino acid sequence having at least 98% identity to the amino acid sequence shown in SEQ ID NO: 6 and an amino acid sequence having at least 98% identity to the amino acid sequence shown in SEQ ID NO: 15;
(c) an amino acid sequence having at least 98% identity to the amino acid sequence shown in SEQ ID NO: 28 and an amino acid sequence having at least 98% identity to the amino acid sequence shown in SEQ ID NO: 15;
(d) an amino acid sequence having at least 98% identity to the amino acid sequence shown in SEQ ID NO: 4 and an amino acid sequence having at least 98% identity to the amino acid sequence shown in SEQ ID NO: 9;
(e) an amino acid sequence having at least 98% identity to the amino acid sequence shown in SEQ ID NO: 9 and an amino acid sequence having at least 98% identity to the amino acid sequence shown in SEQ ID NO: 4;
(f) an amino acid sequence having at least 98% identity to the amino acid sequence shown in SEQ ID NO: 9 and an amino acid sequence having at least 98% identity to the amino acid sequence shown in SEQ ID NO: 30;
(g) an amino acid sequence having at least 98% identity to the amino acid sequence shown in SEQ ID NO: 11 and an amino acid sequence having at least 98% identity to the amino acid sequence shown in SEQ ID NO: 31;
(h) an amino acid sequence having at least 98% identity to the amino acid sequence shown in SEQ ID NO: 11 and an amino acid sequence having at least 98% identity to the amino acid sequence shown in SEQ ID NO: 28;
(i) an amino acid sequence having at least 98% identity to the amino acid sequence shown in SEQ ID NO: 31 and an amino acid sequence having at least 98% identity to the amino acid sequence shown in SEQ ID NO: 15;
(j) an amino acid sequence having at least 98% identity to the amino acid sequence shown in SEQ ID NO: 12 and an amino acid sequence having at least 98% identity to the amino acid sequence shown in SEQ ID NO: 31;
(k) an amino acid sequence having at least 98% identity to the amino acid sequence shown in SEQ ID NO: 12 and an amino acid sequence having at least 98% identity to the amino acid sequence shown in SEQ ID NO: 28;
(l) an amino acid sequence having at least 98% identity to the amino acid sequence shown in SEQ ID NO: 13 and an amino acid sequence having at least 98% identity to the amino acid sequence shown in SEQ ID NO: 31;
(m) an amino acid sequence having at least 98% identity to the amino acid sequence shown in SEQ ID NO: 15 and an amino acid sequence having at least 98% identity to the amino acid sequence shown in SEQ ID NO: 31;
(n) an amino acid sequence having at least 98% identity to the amino acid sequence shown in SEQ ID NO: 15 and an amino acid sequence having at least 98% identity to the amino acid sequence shown in SEQ ID NO: 28; or
(o) an amino acid sequence having at least 98% identity to the amino acid sequence shown in SEQ ID NO: 15 and an amino acid sequence having at least 98% identity to the amino acid sequence shown in SEQ ID NO: 35.

In some embodiments, two lipocalin muteins of the disclosure that are comprised in a fusion protein may comprise the following amino acid sequences, respectively:
(a) an amino acid sequence having at least 99% identity to the amino acid sequence shown in SEQ ID NO: 6 and an amino acid sequence having at least 99% identity to the amino acid sequence shown in SEQ ID NO: 31;
(b) an amino acid sequence having at least 99% identity to the amino acid sequence shown in SEQ ID NO: 6 and an amino acid sequence having at least 99% identity to the amino acid sequence shown in SEQ ID NO: 15;
(c) an amino acid sequence having at least 99% identity to the amino acid sequence shown in SEQ ID NO: 28 and an amino acid sequence having at least 99% identity to the amino acid sequence shown in SEQ ID NO: 15;
(d) an amino acid sequence having at least 99% identity to the amino acid sequence shown in SEQ ID NO: 4 and an amino acid sequence having at least 99% identity to the amino acid sequence shown in SEQ ID NO: 9;
(e) an amino acid sequence having at least 99% identity to the amino acid sequence shown in SEQ ID NO: 9 and an amino acid sequence having at least 99% identity to the amino acid sequence shown in SEQ ID NO: 4;
(f) an amino acid sequence having at least 99% identity to the amino acid sequence shown in SEQ ID NO: 9 and an amino acid sequence having at least 99% identity to the amino acid sequence shown in SEQ ID NO: 30;
(g) an amino acid sequence having at least 99% identity to the amino acid sequence shown in SEQ ID NO: 11 and an amino acid sequence having at least 99% identity to the amino acid sequence shown in SEQ ID NO: 31;
(h) an amino acid sequence having at least 99% identity to the amino acid sequence shown in SEQ ID NO: 11 and an amino acid sequence having at least 99% identity to the amino acid sequence shown in SEQ ID NO: 28;
(i) an amino acid sequence having at least 99% identity to the amino acid sequence shown in SEQ ID NO: 31 and an amino acid sequence having at least 99% identity to the amino acid sequence shown in SEQ ID NO: 15;
(j) an amino acid sequence having at least 99% identity to the amino acid sequence shown in SEQ ID NO: 12 and an amino acid sequence having at least 99% identity to the amino acid sequence shown in SEQ ID NO: 31;
(k) an amino acid sequence having at least 99% identity to the amino acid sequence shown in SEQ ID NO: 12 and an amino acid sequence having at least 99% identity to the amino acid sequence shown in SEQ ID NO: 28;
(l) an amino acid sequence having at least 99% identity to the amino acid sequence shown in SEQ ID NO: 13 and an amino acid sequence having at least 99% identity to the amino acid sequence shown in SEQ ID NO: 31;
(m) an amino acid sequence having at least 99% identity to the amino acid sequence shown in SEQ ID NO: 15 and an amino acid sequence having at least 99% identity to the amino acid sequence shown in SEQ ID NO: 31;
(n) an amino acid sequence having at least 99% identity to the amino acid sequence shown in SEQ ID NO: 15 and an amino acid sequence having at least 99% identity to the amino acid sequence shown in SEQ ID NO: 28; or
(o) an amino acid sequence having at least 99% identity to the amino acid sequence shown in SEQ ID NO: 15 and an amino acid sequence having at least 99% identity to the amino acid sequence shown in SEQ ID NO: 35.

In some embodiments, two lipocalin muteins of the disclosure that are comprised in a fusion protein may comprise the following amino acid sequences, respectively:
(a) the amino acid sequence shown in SEQ ID NO: 6 and SEQ ID NO: 31;
(b) the amino acid sequence shown in SEQ ID NO: 6 and SEQ ID NO: 15;
(c) the amino acid sequence shown in SEQ ID NO: 28 and SEQ ID NO: 15;
(d) the amino acid sequence shown in SEQ ID NO: 4 and SEQ ID NO: 9;
(e) the amino acid sequence shown in SEQ ID NO: 9 and SEQ ID NO: 4;
(f) the amino acid sequence shown in SEQ ID NO: 9 and SEQ ID NO: 30;
(g) the amino acid sequence shown in SEQ ID NO: 11 and SEQ ID NO: 31;
(h) the amino acid sequence shown in SEQ ID NO: 11 and SEQ ID NO: 28;
(i) the amino acid sequence shown in SEQ ID NO: 31 and SEQ ID NO: 15;
(j) the amino acid sequence shown in SEQ ID NO: 12 and SEQ ID NO: 31;
(k) the amino acid sequence shown in SEQ ID NO: 12 and SEQ ID NO: 28;
(l) the amino acid sequence shown in SEQ ID NO: 13 and SEQ ID NO: 31;
(m) the amino acid sequence shown in SEQ ID NO: 15 and SEQ ID NO: 31;
(n) the amino acid sequence shown in SEQ ID NO: 15 and SEQ ID NO: 28; or
(o) the amino acid sequence shown in SEQ ID NO: 15 and SEQ ID NO: 35.

In some embodiments, the present disclosure provides a fusion protein that has at least 75%, at least 80%, at least 85%, preferably at least 90%, preferably at least 95%, preferably at least 98%, preferably at least 99% identity to an amino acid sequence selected from the group consisting of SEQ ID NO: 62-76. In some particular embodiments, a fusion protein of the disclosure comprises an amino acid sequence as set forth in any one of SEQ ID NOs: 62-76 or a fragment or variant thereof. Such a fusion protein preferably binds CTGF with an EC50 value of about 250 nM or lower.

### D. Production the Lipocalin Muteins of the Disclosure.

In one aspect, the disclosure provides methods of making CTGF-binding proteins described herein. The present disclosure also relates to nucleic acid molecules (DNA and RNA) that include nucleotide sequences encoding the lipocalin muteins of the disclosure. In yet another embodiment, the disclosure encompasses a host cell containing said nucleic acid molecule. Since the degeneracy of the genetic code permits substitutions of certain codons by other codons specifying the same amino acid, the disclosure is not limited to a specific nucleic acid molecule encoding a lipocalin mutein as described herein but encompasses all nucleic acid molecules that include nucleotide sequences encoding a functional mutein. In this regard, the present disclosure provides nucleotide sequences encoding some lipocalin muteins of the disclosure as shown in SEQ ID NOs: 3-36.

In one embodiment of the disclosure, the method includes subjecting the nucleic acid molecule encoding mature hNGAL to mutagenesis at nucleotide triplets coding for at least one, or even more, of the sequence positions 28, 36, 40, 41, 44, 47, 49, 52, 65, 70, 72, 73, 74, 75, 77, 79, 80, 81, 87, 94, 95, 96, 97, 98, 99, 100, 102, 103, 104, 106, 110, 123, 125, 127, 128, 129, 130, 132, 134, and 136.

In one embodiment of the disclosure, the method includes subjecting the nucleic acid molecule encoding mature hNGAL to mutagenesis at nucleotide triplets coding for at least one, or even more, of the sequence positions 36, 40, 41, 44, 47, 49, 52, 70, 72, 73, 74, 75, 77, 79, 80, 81, 94, 95, 96, 97, 98, 99, 100, 102, 103, 104, 106, 110, 123, 125, 127, 128, 129, 130, 132, 134, and 136.

In another embodiment of the method according to the disclosure, a nucleic acid molecule encoding mature hNGAL is firstly subjected to mutagenesis at one or more nucleotide triplets coding for the amino acid sequence positions 36, 40, 41, 49, 52, 68, 70, 72, 73, 77, 79, 81, 96, 100, 103, 106, 125, 127, 132, and 134. The nucleic acid molecule may further be subjected to mutagenesis at one or more nucleotide triplets coding for the amino acid sequence positions 44, 47, 74, 75, 80, 94, 95, 97, 98, 99, 102, 104, 110, 123, 128, 129, 130, and 136 of the linear polypeptide sequence of mature hNGAL.

The disclosure also includes nucleic acid molecules encoding the lipocalin muteins or fusion proteins of the disclosure, which include additional mutations outside the indicated sequence positions of experimental mutagenesis. Such mutations are often tolerated or can even prove to be advantageous, for example, if they contribute to an improved folding efficiency, serum stability, thermal stability, formulation stability or ligand binding affinity of the muteins.

A nucleic acid molecule, such as DNA, is referred to as “capable of expressing a nucleic acid molecule” or capable “to allow expression of a nucleotide sequence” if it includes sequence elements which contain information regarding transcriptional and/or translational regulation, and such sequences are “operably linked” to the nucleotide sequence encoding the polypeptide. An operable linkage is a linkage in which the regulatory sequence elements and the sequence to be expressed are connected in a way that enables gene expression. The precise nature of the regulatory regions necessary for gene expression may vary among species, but in general these regions include a promoter, which, in prokaryotes, contains both the promoter per se, i.e., DNA elements directing the initiation of transcription, as well as DNA elements which, when transcribed into RNA, will signal the initiation of translation. Such promoter regions normally include 5′ non-coding sequences involved in the initiation of transcription and translation, such as the −35/−10 boxes and the Shine-Dalgarno element in prokaryotes or the TATA box, CAAT sequences, and 5-capping elements in eukaryotes. These regions can also include enhancer or repressor elements as well as translated signal and leader sequences for targeting the native polypeptide to a specific compartment of a host cell.

In addition, the 3′ non-coding sequences may contain regulatory elements involved in transcriptional termination, polyadenylation or the like. If, however, these termination sequences are not satisfactorily functional in a particular host cell, then they may be substituted with signals functional in that cell.

Therefore, a nucleic acid molecule of the disclosure may be “operably linked” to a regulatory sequence (or regulatory sequences), such as a promoter sequence, to allow expression of this nucleic acid molecule. In some embodiments a nucleic acid molecule of the disclosure includes a promoter sequence and a transcriptional termination sequence. Suitable prokaryotic promoters are, for example, the tet promoter, the lacUV5 promoter or the T7 promoter. Examples of promoters useful for expression in eukaryotic cells are the SV40 promoter or the CMV promoter.

The nucleic acid molecules of the disclosure can be part of a vector or any other kind of cloning vehicle, such as a plasmid, a phagemid, a phage, a baculovirus, a cosmid or an artificial chromosome.

In one embodiment, the nucleic acid molecule is included in a phagemid. A phagemid vector denotes a vector encoding the intergenic region of a temperate phage, such as M13 or f1, or a functional part thereof fused to the cDNA of interest. After superinfection of the bacterial host cells with such a phagemid vector and an appropriate helper phage (e.g., M13K07, VCS-M13 or R408) intact phage particles are produced, thereby enabling physical coupling of the encoded heterologous cDNA to its corresponding polypeptide displayed on the phage surface (Lowman, Annu Rev Biophys Biomol Struct, 1997, 26, 401-24, Rodi and Makowski, Curr Opin Biotechnol, 1999, 10, 87-93).

Such cloning vehicles can include, aside from the regulatory sequences described above and a nucleic acid sequence encoding a lipocalin mutein or a fusion protein as described herein, replication and control sequences derived from a species compatible with the host cell that is used for expression as well as selection markers conferring a selectable phenotype on transformed or transfected cells. Large numbers of suitable cloning vectors are known in the art and are commercially available.

The DNA molecule encoding a lipocalin mutein or a fusion protein as described herein, and in particular a cloning vector containing the coding sequence of such a mutein or fusion protein can be transformed into a host cell capable of expressing the gene. Transformation can be performed using standard techniques. Thus, the disclosure is also directed to a host cell containing a nucleic acid molecule as disclosed herein.

The transformed host cells are cultured under conditions suitable for expression of the nucleotide sequence encoding a fusion protein of the disclosure. Suitable host cells can be prokaryotic, such as Escherichia coli (E. coli) or Bacillus subtilis, or eukaryotic, such as Saccharomyces cerevisiae, Pichia pastoris, SF9 or High5 insect cells, immortalized mammalian cell lines (e.g., HeLa cells or CHO cells) or primary mammalian cells.

The disclosure also relates to a method for the production of a lipocalin mutein, fragment of the mutein, or a fusion protein as described herein, wherein the mutein, a fragment of the mutein or a fusion protein of the mutein and another polypeptide (e.g., another lipocalin mutein or antibody or antibody fragment) is produced starting from the nucleic acid coding for the mutein, fragment, or fusion protein by means of genetic engineering methods. The method can be carried out in vivo, the lipocalin mutein, fragment, or fusion protein can, for example, be produced in a bacterial or eukaryotic host organism and then isolated from this host organism or its culture. It is also possible to produce a protein in vitro, for example by use of an in vitro translation system.

When producing the lipocalin mutein, fragment, or fusion protein in vivo a nucleic acid encoding such mutein, fragment, or fusion protein is introduced into a suitable bacterial or eukaryotic host organism using recombinant DNA technology (as already outlined above). For this purpose, the host cell is first transformed with a cloning vector that includes a nucleic acid molecule encoding a lipocalin mutein, fragment, or fusion protein as described herein using established standard methods. The host cell is then cultured under conditions, which allow expression of the heterologous DNA and thus the synthesis of the corresponding polypeptide. Subsequently, the polypeptide is recovered either from the cell or the cultivation medium.

In addition, in some embodiments, the naturally occurring disulfide bond between Cys 76 and Cys 175 may be removed in hNGAL muteins of the disclosure. Accordingly, such muteins can be produced in a cell compartment having a reducing redox milieu, for example, in the cytoplasm of Gram-negative bacteria.

In case a lipocalin mutein of the disclosure includes intramolecular disulfide bonds, it may be preferred to direct the nascent polypeptide to a cell compartment having an oxidizing redox milieu using an appropriate signal sequence. Such an oxidizing environment may be provided by the periplasm of Gram-negative bacteria such as E. coli, in the extracellular milieu of Gram-positive bacteria or the lumen of the endoplasmic reticulum of eukaryotic cells and usually favors the formation of structural disulfide bonds.

It is, however, also possible to produce a mutein, fragment, or fusion protein of the disclosure in the cytosol of a host cell, preferably E. coli. In this case, the polypeptide can either be directly obtained in a soluble and folded state or recovered in the form of inclusion bodies, followed by renaturation in vitro. A further option is the use of specific host strains having an oxidizing intracellular milieu, which may thus allow the formation of disulfide bonds in the cytosol (Venturi et al., J Mol Biol, 2002, 315, 1-8).

However, a lipocalin mutein, fragment, or fusion protein as described herein may not necessarily be generated or produced only by use of genetic engineering. Rather, such a mutein, fragment, or fusion protein can also be obtained by chemical synthesis such as Merrifield solid phase polypeptide synthesis or by in vitro transcription and translation. It is, for example, possible that promising mutations are identified using molecular modeling, and polypeptides containing such mutations are synthesized in vitro and then investigated for binding activity to CTGF and other desirable properties (such as stability). Methods for the solid phase and solution phase synthesis of polypeptides/proteins are well known in the art (see, e.g., Bruckdorfer et al., Curr Pharm Biotechnol, 2004, 5, 29-43). In another embodiment, the lipocalin mutein, fragment, or fusion protein of the disclosure may be produced by in vitro transcription/translation employing well-established methods known to those skilled in the art.

In addition, the skilled worker will appreciate methods useful to prepare lipocalin mutein, fragment, or fusion protein contemplated by the present disclosure but whose protein or nucleic acid sequences are not explicitly disclosed herein. As an overview, such modifications of the amino acid sequence include, e.g., directed mutagenesis of single amino acid positions to simplify sub-cloning of a mutated lipocalin gene or its parts by incorporating cleavage sites for certain restriction enzymes.

### E. Exemplary Uses and Applications of the Lipocalin Muteins of the Disclosure.

In general, the lipocalin muteins or fusion proteins disclosed herein and their derivatives can be used in many fields similar to antibodies or fragments thereof. For example, the lipocalin muteins or fusion proteins can be used for labeling with an enzyme, an antibody, a radioactive substance or any other group having defined biochemical activity or binding characteristics. By doing so, their respective targets or conjugates or fusion proteins thereof can be detected or brought in contact with them.

Particularly, the disclosure relates to numerous possible applications for the CTGF-binding lipocalin muteins or fusion proteins.

The present disclosure involves the use of one or more CTGF-binding lipocalin muteins or fusion proteins as described herein for complex formation with CTGF.

In one further aspect, the disclosure relates to the use of one or more CTGF-binding lipocalin muteins or fusion proteins disclosed herein for detecting CTGF in a sample as well as a respective method of diagnosis.

Therefore, in another aspect of the disclosure, the disclosed lipocalin muteins or fusion proteins are used for the detection of CTGF. Such use may include the steps of contacting one or more of said muteins or fusion proteins, under suitable conditions, with a sample suspected of containing CTGF, thereby allowing the formation of a complex between the muteins or fusion proteins and CTGF, and detecting the complex by a suitable signal. The detectable signal can be caused by a label, as explained above, or by a change of physical properties due to the binding, i.e., the complex formation, itself. One example is surface plasmon resonance, the value of which is changed during binding of binding partners from which one is immobilized on a surface such as a gold foil.

The CTGF-binding lipocalin muteins or fusion proteins disclosed herein may also be used for the separation of CTGF. Such use may include the steps of contacting one or more of said muteins or fusion proteins, under suitable conditions, with a sample supposed to contain CTGF, thereby allowing formation of a complex between the muteins or fusion proteins and CTGF, and separating the complex from the sample.

In the use of the disclosed muteins or fusion proteins for the detection of CTGF as well as the separation of CTGF, the muteins, fusion proteins and/or CTGF or a domain or fragment thereof may be immobilized on a suitable solid phase.

In still another aspect, the present disclosure features a diagnostic or analytical kit comprising a CTGF-binding lipocalin mutein or fusion protein according to the disclosure.

In addition to their use in diagnostics, in yet another aspect, the disclosure contemplates a pharmaceutical composition comprising a mutein or fusion protein of the disclosure and a pharmaceutically acceptable excipient.

Furthermore, the present disclosure provides human lipocalin muteins and fusion proteins that bind CTGF for use in therapy. As such the lipocalin muteins and fusion proteins of the present disclosure that bind CTGF are envisaged to be used in a method of treatment or prevention of a human disease. Accordingly, also provided are methods of treatment or prevention of human diseases in a subject in need thereof, comprising administering to said subject a therapeutically effective amount of a lipocalin mutein or fusion protein of the present disclosure that binds CTGF. Further, also provided are the use of a lipocalin mutein or fusion protein of the disclosure that binds CTGF for the manufacture of a medicament.

A lipocalin mutein or fusion protein of the disclosure may be used for the treatment of a fibrotic disease, a cancer, an autoimmune disease, or an infectious disease.

A “fibrotic disease” or “fibrosis”, as used interchangeably herein, includes but is not limited to lung fibrosis, such as idiopathic pulmonary fibrosis (IPF) or interstitial lung disease (ILD) (e.g., progressive fibrosing ILD (PF-ILD), cardiac fibrosis, liver fibrosis and kidney fibrosis. In some embodiments, the fibrosis is radiation-induced fibrosis (RIF), such as radiation-induced lung fibrosis. In some embodiments, a lipocalin mutein or fusion protein of the disclosure is used to treat IPF or PF-ILD.

A cancer that may be treated with a lipocalin mutein or fusion protein of the disclosure includes but is not limited to breast cancer, chondrosarcomas, enchondroma, glioma, pancreatic cancer, thyroid cancer, intrahepatic cholangiocarcinoma, neuroendocrine tumors, and squamous cell carcinoma of the tongue.

An autoimmune disease that may be treated with a lipocalin mutein or fusion protein of the disclosure includes but is not limited to systemic sclerosis.

An infectious disease that may be treated with a lipocalin mutein or fusion protein of the disclosure includes but is not limited to an infectious disease of the respiratory tract, such as pneumonia. Treatment of the infectious disease may include the treatment or prevention of lung damages in connection with, e.g., concomitant with or following, the infectious disease. The infectious disease may be a coronavirus infection, such as SARS, MERS, or COVID-19. Treatment of COVID-19 may include the treatment of acute COVID-19, ongoing symptomatic COVID-19, post-COVID-19 syndrome (also referred to as “long COVID” or “post-acute sequelae of COVID-19 (PASC)”), and the treatment of organ damages concomitant with or following COVID-19 infection, such as lung damage or heart damage. In some embodiments, a lipocalin mutein or fusion protein of the disclosure is used to treat post-COVID-19 syndrome, in particular post-COVID-19 syndrome pulmonary fibrosis (also referred to as “PASC-PF”).

A lipocalin mutein or fusion protein of the disclosure may be used for the treatment of a lung disease, such as lung fibrosis, a muscle disease, such as muscular dystrophy, such as Duchenne muscular dystrophy, a heart disease, a liver disease, a kidney disease, or an eye disease, such as (diabetic) retinopathy or glaucoma.

The present disclosure also provides a lipocalin mutein or fusion protein of the disclosure that binds CTGF for inhibiting fibrogenesis or for inhibiting (pathological) deposition of extracellular matrix.

The present disclosure encompasses the use of a CTGF-binding lipocalin mutein or fusion protein of the disclosure or a composition comprising such lipocalin mutein or fusion protein for regulating downstream signaling pathways of CTGF. Such use may comprise binding of CTGF.

The present disclosure thus features a method of providing an anti-fibrotic effect in vivo, comprising applying one or more CTGF-binding lipocalin muteins or fusion proteins of the disclosure or one or more compositions comprising such lipocalin muteins or fusion proteins.

Furthermore, the present disclosure involves a method of regulating downstream signaling pathways of CTGF, comprising applying one or more CTGF-binding lipocalin muteins or fusion proteins of the disclosure or one or more compositions comprising such lipocalin muteins or fusion proteins.

The present disclosure also contemplates a method of reducing collagen deposition in the lung, such as Collagen 1a1 (COL1a1) deposition, comprising applying one or more CTGF-binding lipocalin muteins or fusion proteins of the disclosure or one or more compositions comprising such lipocalin muteins or fusion proteins.

### F. Administration of Lipocalin Muteins of the Disclosure

A lipocalin mutein or fusion protein disclosed herein can be administered to a subject by any suitable mode of administration. Suitable modes of administration may include but are not limited to enteral and parenteral routes. Suitable administration routes may include but are not limited to oral administration, intranasal administration, administration to a mucosal surface, inhalation, intradermal administration, intraperitoneal administration, subcutaneous administration, intravenous administration, or intramuscular administration.

A lipocalin mutein or fusion protein of the disclosure may be administered by inhalation. Means and devices for inhaled administration of a substance are known to the skilled person and are for example disclosed in WO 94/017784A and Elphick et al. (2015) Expert Opin. Drug Deliv., 12(8):1375-87. Such means and devices include nebulizers, metered dose inhalers, powder inhalers, and nasal sprays. Other means and devices suitable for directing inhaled administration of a lipocalin mutein or fusion protein are also known in the art. Nebulizers are useful in producing aerosols from solutions, while metered dose inhalers, dry powder inhalers, etc. are effective in generating small particle aerosols.

A nebulizer is a drug delivery device used to administer medication in the form of a mist inhaled into the lungs. Different types of nebulizers are known to the skilled person and include jet nebulizers, ultrasonic wave nebulizers, vibrating mesh technology, and soft mist inhalers. Some nebulizers provide a continuous flow of nebulized solution, i.e., they will provide continuous nebulization over a long period of time, regardless of whether the subject inhales from it or not, while others are breath-actuated, i.e., the subject only gets some dose when they inhale from it. In some embodiments, a lipocalin mutein or fusion protein of the disclosure is administered via a vibrating mesh nebulizer.

A metered-dose inhaler (MDI) is a device that delivers a specific amount of medication to the lungs, in the form of a short burst of liquid aerosolized medicine. Such a metered-dose inhaler commonly consists of three major components; a canister which comprises the formulation to be administered, a metering valve, which allows a metered quantity of the formulation to be dispensed with each actuation, and an actuator (or mouthpiece) which allows the patient to operate the device and directs the liquid aerosol into the patient's lungs.

A dry-powder inhaler (DPI) is a device that delivers medication to the lungs in the form of a dry powder. Dry powder inhalers are an alternative to the aerosol-based inhalers, such as metered-dose inhalers. The medication is commonly held either in a capsule for manual loading or a proprietary blister pack located inside the inhaler.

Nasal sprays can be used for nasal administration, by which a drug is insufflated through the nose. Nasal sprays may provide extremely quick absorption of the medication.

Additional objects, advantages, and features of this disclosure will become apparent to those skilled in the art upon examination of the following Examples and the attached Figures thereof, which are not intended to be limiting. Thus, it should be understood that although the present disclosure is specifically disclosed by exemplary embodiments and optional features, modifications and variations of the disclosures may be resorted to by those skilled in the art, and that such modifications and variations are considered to be within the scope of this disclosure.

### V. EXAMPLES

### Example 1: Selection and Optimization of Muteins Specific to CTGF

The CTGF-specific lipocalin muteins disclosed in this application were selected from naïve mutant libraries based on hNGAL. The libraries were panned against recombinant human CTGF/CNN2 protein and recombinant rat CTGF protein (R&D Systems and in-house biotinylated). Protein-based pannings were performed using standard procedures. The clones obtained after selection were subjected to a screening process as described in Example 2.

For optimization of CTGF-specific muteins, DNA-encoded libraries of lipocalin muteins were generated based on muteins SEQ ID NOs: 3, 7, 9, 25, and 29 using either a randomization of selected positions or error prone polymerase chain reaction (PCR) based methods. The generated lipocalin muteins were cloned with high efficiency into phagemid vector essentially as described (Kim et al., 2009, J. Am. Chem. Soc., 131, 10, 3565-3576). Phage display was employed to select for optimized muteins with improved heat stability and binding affinity. The phagemid selection was conducted against recombinant human CTGF/CNN2 protein and recombinant rat CTGF protein (R&D Systems and in-house biotinylated), under increased stringency compared to the initial mutein selections and involved preincubation steps at elevated temperature and limiting target concentration amongst other things.

### Example 2: Identification of Muteins Specifically Binding to CTGF Using High-Throughput Enzyme-Linked Immunosorbent Assay (ELISA) Screening

Individual colonies of lipocalin muteins genetically fused to a C-terminal Strep-tag II (SEQ ID NO: 41) were used to inoculate 2× Yeast Extract Trypton (2×YT)/Amp medium and grown overnight (14-18 h) to stationary phase. Subsequently, 50 μL 2×YT/Amp were inoculated from the stationary phase cultures and incubated for 3.5-5.5 h at 37° C. and then shifted to 22° C. until an OD₅₉₅ of 0.6-0.8 was reached. Production of muteins was induced by addition of 10 μL 2×YT/Amp supplemented with 1.2 μg/mL anhydrotetracycline. Cultures were incubated at 22° C. until the next day. After addition of 40 μL of 5% (w/v) BSA in PBS/T and incubation for 1 h at 25° C., cultures were ready for use in screening assays.

Binding of the isolated muteins to CTGF was tested by direct coating of recombinant human CTGF protein (SEQ ID NO: 79), recombinant rat CTGF protein (R&D Systems, SEQ ID NO: 82) and recombinant human CTGF-Fc protein (Creative Biomart, SEQ ID NO: 83) at 2 μg/mL in PBS overnight at 4° C. on microtiter plates. After blocking the plate with PBST containing 5% BSA, 20 μL of BSA-blocked cultures were added to the microtiter plates and incubated for 1 h at room temperature. Bound muteins were detected with anti-StrepTag antibody conjugated with horseradish peroxidase (HRP) (IBA Lifesciences) after 1 h incubation. For quantification, 20 μL of QuantaBlu fluorogenic peroxidase substrate was added and the resulting fluorescence was determined at an excitation wavelength of 330 nm and an emission wavelength of 420 nm.

To select for muteins with increased affinity and stability the screening was performed with i) reduced antigen concentration (using recombinant human CTGF/CNN2 protein and recombinant rat CTGF protein (R&D Systems and in-house biotinylated), ii) using reverse screening formats where the muteins were captured via the Strep-tag on microtiter plates coated with anti-Strep-Tag antibody and 2.5 nM of biotinylated recombinant human CTGF/CNN2 protein was added and detected via Extravidin-HRP (Sigma) and partially iii) incubation of the screening supernatant at 65-75° C. before addition to the target plate.

Clones were then sequenced based on the screening results, and muteins were selected for further characterization.

### Example 3: Expression of Muteins

Selected muteins with C-terminal sequence SAWSHPQFEK (SEQ ID NO: 40) of SA linker and the Strep-tag II peptide (WSHPQFEK, SEQ ID NO: 41) were expressed in E. coli in 2×YT/Amp medium and purified using Strep-Tactin affinity chromatography and preparative size exclusion chromatography (SEC). After SEC purification, the fractions containing monomeric protein are pooled and analyzed again using analytical SEC. The yield of exemplary lipocalin muteins after Strep-Tactin affinity chromatography and preparative SEC is shown in Table 1 as well as the monomer content after Strep-Tactin purification.

Some of the lipocalin muteins and fusion proteins were expressed in CHO with a C-terminal His-tag using state of the art techniques (see Table 2).

TABLE 1
Expression of muteins in E. coli.

| | | Monomer content after |
|---|---|---|
| SEQ ID | Yield after SEC | affinity purification |
| NO: | purification [mg/L] | (assessed by analytical SEC) [%] |
| 3 | 0.6 | 98 |
| 7 | 3.75 | 96 |
| 9 | 1.7 | 97 |
| 23 | 6.5 | 97 |
| 24 | 7.8 | 97 |
| 25 | 3.3 | 94 |
| 29 | 4.0 | 94 |
| 34 | 3.5 | 96 |
| 36 | 4.1 | 93 |

TABLE 2
Expression of muteins and fusion proteins in CHO cells.

| | | Monomer content after |
|---|---|---|
| SEQ ID | Yield after IMAC | affinity purification |
| NO: | purification [mg/L] | (assessed by analytical SEC) [%] |
| 5 | 4.3 | 94.4 |
| 6 | 2.9 | 97.1 |
| 8 | 2.7 | 95.5 |
| 10 | 12.1 | 88.9 |
| 11 | 11.8 | 98.0 |
| 12 | 8.5 | 73.3 |
| 13 | 7.8 | 71.7 |
| 14 | 3.3 | 88.8 |
| 15 | 4.7 | 92.8 |
| 16 | 3.6 | 92.8 |
| 17 | 5.1 | 90.9 |
| 18 | 8.7 | 99.0 |
| 19 | 7.3 | 87.1 |
| 20 | 4.3 | 85.9 |
| 21 | 6.4 | 86.9 |
| 22 | 1.9 | 90.0 |
| 23 | 2.4 | 93.3 |
| 24 | 3.5 | 94.1 |
| 26 | 13.1 | 87.2 |
| 27 | 4.0 | 89.0 |
| 28 | 2.5 | 86.7 |
| 31 | 9.3 | 94.3 |
| 32 | 6.3 | 93.6 |
| 33 | 6.7 | 94.9 |
| 34 | 9.7 | 94.7 |
| 35 | 9.2 | 95.8 |
| 36 | 6.2 | 93.8 |
| 62 | 4.7 | 97.1 |
| 63 | 3.4 | 95.4 |
| 64 | 5.2 | 92.9 |
| 68 | 6.2 | 91.1 |
| 69 | 5.6 | 88.1 |
| 70 | 5.3 | 97.5 |
| 71 | 5.6 | 98.4 |
| 72 | 5.4 | 90.9 |
| 73 | 6.2 | 98.6 |
| 74 | 5.3 | 97.3 |
| 75 | 4.7 | 87.3 |
| 76 | 5.3 | 97.3 |

### Example 4: Thermal Stability Assessment of Lipocalin Muteins

To determine the melting temperatures (Tₘs) of the lipocalin muteins, which is a general indicator for folding stability, the CTGF specific muteins, at a protein concentration of 1 mg/mL in PBS (Gibco), were scanned (25-100° C.) at 1° C./min using a capillary nanoDSC instrument (CSC 6300, TA Instruments). The Tₘs were calculated from the displayed thermogram using the integrated Nano Analyze software. Several unfolding events can contribute to the observed thermogram. In cases with multiple transitions, the one with the major unfolding energy best represents the overall protein fold and is reported. Muteins were produced as described in Example 3. SEQ ID NOs: 3, 7, 9, 25, and 29 were expressed using E. coli, the other muteins were expressed using CHO cells.

The resulting maximum melting temperatures as well as the onset temperature of melting for exemplary lipocalin muteins and fusion proteins are listed in Table 3 below.

TABLE 3
Tₘ and onset melting temperature as determined
by nanoDSC of CTGF-specific lipocalin muteins.

| SEQ ID NO: | T_{M} [° C.] | ONSET MELTING [° C.] |
|---|---|---|
| 3 | 58 | n.d. |
| 5 | 66 | 52 |
| 6 | 66 | 48 |
| 7 | 65 | n.d. |
| 8 | 75 | 49 |
| 9 | 64 | n.d. |
| 10 | 66 | 50 |
| 14 | 68 | 57 |
| 15 | 73 | 59 |
| 16 | 73 | 59 |
| 17 | 74 | 59 |
| 19 | 65 | 49 |
| 20 | 61 | 46 |
| 21 | 65 | 55 |
| 22 | 73 | 59 |
| 23 | 71 | 55 |
| 24 | 72 | 58 |
| 25 | 66 | n.d. |
| 26 | 71 | 62 |
| 27 | 64 | 52 |
| 28 | 65 | 55 |
| 29 | 76 | n.d. |
| 31 | 75 | 59 |
| 32 | 79 | 62 |
| 33 | 72 | 56 |
| 34 | 82 | 67 |
| 35 | 78 | 63 |
| 36 | 73 | 58 |
| 62 | 67 | 53 |
| 63 | 66 | 50 |
| 64 | 65 | 56 |
| 68 | 72 | 61 |
| 69 | 64 | 57 |
| 70 | 73 | 55 |
| 71 | 76 | 67 |
| 72 | 66 | 62 |
| 73 | 75 | 71 |
| 74 | 72 | 62 |
| 75 | 65 | 58 |
| 76 | 73 | 63 |

### Example 5: Affinity of Muteins Binding to Human, Cynomolgus, Rat, and Murine CTGF Determined by Surface Plasmon Resonance (SPR)

Surface plasmon resonance (SPR) was used to measure binding kinetics and affinity of the representative lipocalin muteins disclosed herein.

The binding of exemplary lipocalin muteins to human (Creative Biomart), cynomolgus, rat (R&D Systems) and mouse CTGF (Abbexa) was determined by SPR using a Biacore instrument (Cytiva, formerly GE Healthcare). Some targets were fused to a human IgG1 Fc tag, and captured via the anti-human IgG antibody kit, others were directly immobilized.

The anti-human IgG Fc antibody (GE Healthcare) was immobilized on a CM5 sensor chip using standard amine chemistry according to the manufacture's instruction and resulted in an immobilization level of 5500-14000 resonance units (RU). For some measurements, human CTGF with a human IgG1 Fc Tag or rat or murine CTGF without a tag were directly immobilized to a CM5 Chip. The carboxyl groups on the chip were activated using 1-ethyl-3-(3-dimethylaminopropyl)-carbodiimide (EDC) and N-hydroxysuccinimide (NHS). Subsequently, the targets at a concentration of 3-5 μg/mL in 10 mM sodium acetate (pH) 4.5 were applied at a flow rate of 10 μL/min until an immobilization level of 300-1100 resonance units was achieved. Residual non-reacted NHS-esters were blocked by passing a solution of 1 M ethanolamine across the surface. The reference channel was activated/deactivated. For the capture setting, targets with an IgG Fc Tag at 0.25-2.5 μg/mL in HBS-EP+ buffer were captured by the anti-human IgG-Fc antibody at the chip surface for 180 s at a flow rate of 10 μL/min.

For affinity determination of the lipocalin muteins, dilutions of each mutein at various concentrations, typically ranging from 0.43-2000 nM, were prepared in HBS-EP+ buffer and applied to the prepared chip surface for affinity measurement. The binding assay was carried out with a contact time of 180 s, a dissociation time of 900-3600 s and a flow rate of 30 μL/min. All measurements were performed at 25° C. Lipocalin muteins that do not bind to CTGF were also tested as a negative control. Regeneration of the Fc capture chip surface was achieved with injections of 3 M MgCl₂ for 60-120 s and 10 mM glycine-HCl (pH 1.7) for 60-120 s at a flow rate of 10 μL/min followed by an extra wash with running buffer (HBS-EP+ buffer) and a stabilization period of 120 s. For the directly immobilized targets, the same regeneration conditions were used. Prior to the protein measurements, three startup cycles were performed for conditioning purposes. Data were evaluated with Biacore Evaluation software. Double referencing was used and the 1:1 binding model was used to fit the raw data.

The values determined for kₒₙ, k_{off} and the resulting equilibrium dissociation constant (K_{D}) for exemplary lipocalin muteins are summarized in Table 4. The binding affinities to human and cynomolgus CTGF are comparable for most of the tested lipocalin muteins, representing a preferred feature for pharmacokinetic and/or drug-safety studies. Optimized lipocalin muteins SEQ ID NOs: 4-6, 8, 10-24, 26-28, 30-36 have K_{D} values in the low nano-molar range, exhibiting up to 1500-fold lower K_{D} values compared to the parent lipocalin muteins SEQ ID NO: 3, 7, 9, 25, and 29. The K_{D} values of several optimized lipocalin muteins were also significantly lower than that of the anti-CTGF monoclonal antibody of SEQ ID NOs: 60 and 61 (K_{D}-0.2 nM for human CTGF). Furthermore, several optimized lipocalin muteins exhibited a slower dissociation rate as compared to the antibody (k_{off}˜2.1 E-04 s⁻¹ for human CTGF), indicating prolonged target engagement of the lipocalin muteins.

TABLE 4
Kinetic constants and affinities of CTGF-specific muteins determined by surface-plasmon-resonance (SPR).

| | human CTGF | | | cynomolgus CTGF | | | Murine CTGF | | | Rat CTGF | | |
|---|---|---|---|---|---|---|---|---|---|---|---|---|
| SEQ | kₒₙ | k_{off} | K_{D} | kₒₙ | k_{off} | K_{D} | kₒₙ | k_{off} | K_{D} | kₒₙ | k_{off} | K_{D} |
| ID NO: | [M⁻¹ · s⁻¹] | [s⁻¹] | [nM] | [M⁻¹ · s⁻¹] | [s⁻¹] | [nM] | [M⁻¹ · s⁻¹] | [s⁻¹] | [nM] | [M⁻¹ · s⁻¹] | [s⁻¹] | [nM] |
| 3 | 2.00E+05 | 7.49E−03 | 37.500 | n.t. | n.t. | n.t. | B | B | B | 2.97E+05 | 1.17E−02 | 39.300 |
| 5 | 2.11E+05 | 6.65E−05 | 0.315 | n.t. | n.t. | n.t. | B | B | B | 2.94E+05 | 3.62E−04 | 1.233 |
| 6 | 2.90E+05 | 1.49E−04 | 0.514 | n.t. | n.t. | n.t. | B | B | B | B | B | B |
| 7 | 9.76E+04 | 5.87E−03 | 60.200 | n.t. | n.t. | n.t. | B | B | B | 1.20E+05 | 7.54E−03 | 62.800 |
| 8 | 4.54E+04 | 1.14E−04 | 2.516 | n.t. | n.t. | n.t. | 7.20E+04 | 1.31E−04 | 1.815 | n.t. | n.t. | n.t. |
| 9 | 7.98E+04 | 1.02E−03 | 12.811 | n.t. | n.t. | n.t. | 1.33E+05 | 8.32E−04 | 6.264 | 1.05E+05 | 1.69E−03 | 16.000 |
| 10 | 6.77E+04 | 2.23E−06 | 0.033 | n.t. | n.t. | n.t. | B | B | B | 8.88E+04 | 4.08E−05 | 0.460 |
| 11 | 4.44E+04 | 1.08E−04 | 2.437 | n.t. | n.t. | n.t. | 8.01E+04 | 8.19E−05 | 1.022 | n.t. | n.t. | n.t. |
| 12 | 5.56E+04 | 1.33E−04 | 2.397 | n.t. | n.t. | n.t. | 9.14E+04 | 1.28E−04 | 1.405 | n.t. | n.t. | n.t. |
| 13 | 5.10E+04 | 2.51E−06 | 0.049 | n.t. | n.t. | n.t. | B | B | B | n.t. | n.t. | n.t. |
| 14 | 1.02E+05 | 5.34E−05 | 0.523 | n.t. | n.t. | n.t. | 1.90E+05 | 7.03E−05 | 0.369 | n.t. | n.t. | n.t. |
| 15 | 8.17E+04 | 4.47E−05 | 0.547 | 1.07E+05 | 7.57E−05 | 0.707 | 1.29E+05 | 4.51E−05 | 0.349 | 1.20E+05 | 1.14E−04 | 0.950 |
| 16 | 9.23E+04 | 7.02E−05 | 0.760 | n.t. | n.t. | n.t. | 1.61E+05 | 6.34E−05 | 0.393 | n.t. | n.t. | n.t. |
| 17 | B | B | B | n.t. | n.t. | n.t. | B | B | B | n.t. | n.t. | n.t. |
| 18 | 4.09E+04 | 3.05E−06 | 0.074 | n.t. | n.t. | n.t. | B | B | B | n.t. | n.t. | n.t. |
| 19 | 5.66E+04 | 5.20E−05 | 0.918 | n.t. | n.t. | n.t. | 9.32E+04 | 2.54E−05 | 0.273 | n.t. | n.t. | n.t. |
| 20 | B | B | B | n.t. | n.t. | n.t. | B | B | B | n.t. | n.t. | n.t. |
| 21 | 1.27E+05 | 6.63E−05 | 0.523 | n.t. | n.t. | n.t. | 2.28E+05 | 3.94E−05 | 0.173 | n.t. | n.t. | n.t. |
| 22 | 1.42E+05 | 8.05E−06 | 0.057 | n.t. | n.t. | n.t. | B | B | B | 1.81E+05 | 4.01E−05 | 0.221 |
| 23 | 1.22E+05 | 9.42E−06 | 0.077 | B | B | B | B | B | B | n.t. | n.t. | n.t. |
| 24 | 1.57E+05 | 1.41E−05 | 0.090 | 1.95E+05 | 2.63E−05 | 0.135 | 2.99E+05 | 1.16E−05 | 0.034 | 1.38E+05 | 3.51E−05 | 0.255 |
| 25 | 2.68E+04 | 2.48E−04 | 9.270 | n.t. | n.t. | n.t. | No | No | No | 4.65E+04 | 1.29E−03 | 27.700 |
| | | | | | | | binding | binding | binding | | | |
| 26 | 2.32E+04 | 3.06E−05 | 1.320 | n.t. | n.t. | n.t. | 3.53E+04 | 2.08E−04 | 5.895 | n.t. | n.t. | n.t. |
| 27 | 3.05E+04 | 2.45E−05 | 0.802 | n.t. | n.t. | n.t. | 4.14E+04 | 1.72E−04 | 4.161 | n.t. | n.t. | n.t. |
| 28 | 5.04E+04 | 2.61E−05 | 0.517 | n.t. | n.t. | n.t. | 6.07E+04 | 8.23E−05 | 1.355 | n.t. | n.t. | n.t. |
| 29 | 1.00E+05 | 1.12E−03 | 11.200 | n.t. | n.t. | n.t. | No | No | No | 1.76E+05 | 3.17E−03 | 18.000 |
| | | | | | | | binding | binding | binding | | | |
| 31 | 1.95E+05 | 4.85E−05 | 0.249 | 1.62E+05 | 7.31E−05 | 0.452 | 2.81E+05 | 4.44E−04 | 1.579 | 2.23E+05 | 4.23E−04 | 1.891 |
| 32 | 1.09E+05 | 6.39E−05 | 0.587 | n.t. | n.t. | n.t. | 1.33E+05 | 2.64E−04 | 1.976 | n.t. | n.t. | n.t. |
| 33 | 1.72E+05 | 1.71E−05 | 0.099 | n.t. | n.t. | n.t. | 1.76E+05 | 5.29E−05 | 0.301 | 2.79E+05 | 6.30E−05 | 0.226 |
| 34 | 1.50E+05 | 1.69E−04 | 1.129 | 1.39E+05 | 2.38E−04 | 1.717 | 2.40E+05 | 2.86E−03 | 11.910 | n.t. | n.t. | n.t. |
| 35 | 1.08E+05 | 5.94E−05 | 0.550 | n.t. | n.t. | n.t. | 1.28E+05 | 2.87E−04 | 2.244 | 1.93E+05 | 2.73E−04 | 1.418 |
| 36 | 1.65E+05 | 6.95E−05 | 0.420 | 1.41E+05 | 9.77E−05 | 0.6950 | 3.10E+05 | 5.20E−04 | 1.679 | n.t. | n.t. | n.t. |

n.t. not tested, B binding without determination of kinetics constants.

### Example 6: ELISA of Fusion Proteins

Binding of fusion proteins was tested by an ELISA assay. In detail, a 384-well plate suitable for fluorescence measurements (Greiner FLUOTRAC™ 600, black flat bottom, high-binding) was coated with 20 μl of human CTGF (R&D Systems) at a concentration of 1 μg/ml in PBS over night at 4° C. After washing with PBS with 0.05% Tween 20, wells were blocked with 100 μl blocking buffer containing 0.1% Tween 20 and 2% BSA (PBS-T/BSA) for 1 h at room temperature. 20 μl of serially diluted muteins were incubated in PBS-T/BSA for 1 h at room temperature (RT).

The residual supernatants were discarded and 20 μl HRP-labeled anti-NGAL or anti-His-Tag antibody was added at a predetermined optimal concentration in PBS-T/BSA and incubated for 1 h at RT. After washing, 20 μl fluorogenic HRP substrate (QuantaBlu, Thermo) was added to each well, and the reaction was allowed to proceed for 2 to 60 minutes. The fluorescence intensity of every well on the plate was read using a fluorescence microplate reader (Tecan). Curve fitting was performed using GraphPad Prism 7 or 8 software. The resulting EC50 values are summarized in Table 5 below.

TABLE 5
EC50 values in ELISA assays.

| SEQ ID NO: | EC50 [nM] |
|---|---|
| 65 | 0.61 |
| 66 | 1.20 |
| 67 | 1.00 |
| 74 | 0.26 |
| 76 | 0.24 |

### Example 7: HTRF Assay of Fusion Proteins

Binding of some fusion proteins was tested by a Homogeneous Time Resolved Fluorescence (HTRF assay). In detail, 1 nM of biotinylated human CTGF (R&D Systems) was incubated for 1 h with a titration of some lipocalins starting at 5 nM in 384 Flat Bottom White Polystyrol microtiter plate (Greiner). Samples were diluted in PBS containing 0.1% Tween 20 and 2% BSA. As donor, Streptavidin-Terbium at a concentration of 0,006 μg/mL was used, the acceptor was 0.2 μg/mL anti-His-d2 (both CisBio). After another 1 h of incubation, the fluorescence intensity of the donor and acceptor on the plate was read using a fluorescence microplate reader M1000 (Tecan) with the standard settings recommended by CisBio. The calculated ratio of signals was used for the curve fitting performed in GraphPad Prism 7 software. The resulting EC50 values are summarized in Table 6 below.

TABLE 6
EC50 values in HTRF assays.

| SEQ ID NO: | EC50 [nM] |
|---|---|
| 62 | 0.63 |
| 63 | 0.58 |
| 64 | 0.58 |
| 68 | 0.56 |
| 69 | 0.65 |
| 70 | 0.72 |
| 71 | 0.94 |
| 72 | 0.68 |
| 73 | 0.83 |
| 74 | 0.59 |
| 75 | 0.70 |
| 76 | 0.62 |

### Example 8: Epitope Analysis of the Lipocalin Muteins

Surface plasmon resonance (SPR) was used to analyze the epitopes and determine simultaneous binding of different lipocalin muteins. Human CTGF with a human IgG1 Fc Tag was directly immobilized to a CM5 Chip (Creative Biomart, 10 μg/mL in 10 mM sodium acetate pH 4.5) until an immobilization level of 470-630 resonance units was achieved. The reference channel was activated/deactivated. 2 different lipocalin muteins or the antibody shown in SEQ ID NOs: 60 and 61 were mixed and injected simultaneously at 750 nM or 1000 nM for 180 s at a flow rate of 30 μg/mL. As control, single lipocalin muteins were injected and the resulting signal of single and mixtures was compared at the end of the injection. The chip was regenerated with 3 M MgCl2 and Glycine-HCl pH1.5 for each 60 s at a flowrate of 15 μL/min.

Table 7 summarizes the results. “Yes” means simultaneous binding is possible, “no” stands for same or overlapping epitope, “n.t.” stands for not tested.

TABLE 7
Simultaneous binding of various lipocalin
muteins and control antibody.

| SEQ | | | | | | 60 |
|---|---|---|---|---|---|---|
| ID NO: | 9 | 29 | 7 | 25 | 3 | and 61 |
| 9 | no | yes | no | yes | n.t. | no |
| 29 | yes | no | yes | no | n.t. | yes |
| 7 | no | yes | no | yes | n.t. | no |
| 25 | yes | no | yes | no | n.t. | yes |
| 3 | n.t. | n.t. | n.t. | n.t. | no | yes |
| 60 and 61 | no | yes | no | yes | yes | no |

The epitope of the exemplary lipocalin mutein of SEQ ID NO: 23 also overlapped with that of the anti-CTGF monoclonal antibody of SEQ ID NOs: 60 and 61, and the lipocalin mutein competed with the antibody for binding to CTGF in an ELISA-based competition assay (data not shown).

### Example 9: Binding to CTGF Fragments

Binding of muteins to fragments of human and murine CTGF was tested by an ELISA assay. In detail, a 384-well plate suitable for fluorescence measurements (Greiner FLUOTRAC™ 600, black flat bottom, high-binding) was coated with 20 μl of human (R&D Systems, SEQ ID NO 79) and murine CTGF (Biozol, SEQ ID NO: 80) or fragments thereof (Evitria, huCTGF domains 1 and 2 with human Fc tag (SEQ ID NO 77) and muCTGF domains 1 and 2 with human Fc tag (SEQ ID NO 78)) at a concentration of 1 μg/ml in PBS over night at 4° C. After washing with PBS with 0.05% Tween 20, wells were blocked with 100 μl blocking buffer containing 0.1% Tween 20 and 2% BSA (PBS-T/BSA) for 1 h at room temperature. 20 μl of serially diluted muteins starting at 1000 nM were incubated in PBS-T/BSA for 1 h at room temperature.

The residual supernatants were discarded and 20 μl HRP-labeled anti-Strep-Tag antibody was added at a predetermined optimal concentration in PBS-T/BSA and incubated for 1 h at RT. After washing, 20 μl fluorogenic HRP substrate (QuantaBlu, Thermo) was added to each well, and the reaction was allowed to proceed for 5 to 25 minutes. The fluorescence intensity of every well on the plate was read using a fluorescence microplate reader (Tecan). Curve fitting was performed using GraphPad Prism 7 software. The resulting EC50 values are summarized in Table 8 below.

TABLE 8
EC50 values in ELISA assays.

| | EC [nM] | | | | |
|---|---|---|---|---|---|
| | Murine | Human | | | |
| SEQ ID | CTGF1-2- | CTGF1-2- | Human | Murine | Rat |
| NO: | huFc | huFc | CTGF | CTGF | CTGF |
| 3 | n.b. | n.b. | 6.9 | n.b. | weak |
| 7 | 14 | 17 | 34 | 29 | 41 |
| 9 | 5.8 | 8 | 14 | 11 | 30 |
| 25 | 14 | 8.8 | 11 | n.b. | 23 |
| 29 | 17 | 8.4 | 11 | n.b. | 20 |

### Example 10: Specificity ELISA (CCN Proteins)

Binding of muteins to closely related proteins of CTGF was tested by an ELISA assay. In detail, a 384-well plate suitable for fluorescence measurements (Greiner FLUOTRAC™ 600, black flat bottom, high-binding) was coated with 20 μl of human CTGF or the other members of the CCN protein family at a concentration of 1 μg/ml in PBS over night at 4° C. After washing with PBS with 0.05% Tween 20, wells were blocked with 100 μl blocking buffer containing 0.1% Tween 20 and 2% BSA (PBS-T/BSA) for 1 h at room temperature. 20 μl of serially diluted muteins starting at 1000 nM were incubated in PBS-T/BSA for 1 h at room temperature (RT).

The residual supernatants were discarded and 20 μl HRP-labeled anti-NGAL or anti-Strep-Tag antibody was added at a predetermined optimal concentration in PBS-T/BSA and incubated for 1 h at RT. After washing, 20 μl fluorogenic HRP substrate (QuantaBlu, Thermo) was added to each well, and the reaction was allowed to proceed for 2 to 60 minutes. The fluorescence intensity of every well on the plate was read using a fluorescence microplate reader (Tecan). When the signal for human CTGF approached saturation, the cross reactivity to the other proteins was assessed.

TABLE 9
Results of ELISA assays.

| SEQ | HUMAN | | | | | |
|---|---|---|---|---|---|---|
| ID NO: | CTGF | huCYR61 | huNOV | huWISP-1 | huWISP-2 | huWISP-3 |
| 3 | Binding | No binding | No binding | No binding | No binding | No binding |
| 7 | Binding | No binding | No binding | No binding | No binding | No binding |
| 9 | Binding | No binding | No binding | No binding | No binding | No binding |
| 25 | Binding | No binding | No binding | No binding | No binding | No binding |
| 29 | Binding | No binding | No binding | No binding | No binding | No binding |
| 23 | Binding | No binding | No binding | No binding | No binding | No binding |
| 15 | Binding | No binding | No binding | No binding | No binding | No binding |
| 74 | Binding | No binding | No binding | No binding | No binding | No binding |
| 76 | Binding | No binding | No binding | No binding | No binding | No binding |

### Example 11: Anti-Fibrotic Effect of CTGF-Targeting Lipocalin Muteins in the Bleomycin Mouse Model of Lung Fibrosis In Vivo

The anti-fibrotic activity of lipocalin muteins upon local delivery to the lung was evaluated in vivo using a mouse model of bleomycin-induced lung fibrosis. Bleomycin is a DNA damaging agent, also used in cancer therapy, which induces a severe damage of the lung epithelium upon delivery to the lungs of rodents. The injury causes a strong inflammatory response in the lungs which in turn triggers fibrotic response. The peak of fibrotic remodeling, usually observed between day 14 and 21 after bleomycin challenge, is characterized by an excessive deposition of extracellular matrix and destruction of alveolar lung structures. Here, approximately 9 weeks old, male C57BL/6N mice were challenged with bleomycin at a dose of 1 mg/kg or saline as a control via the intranasal route. Animals were treated daily with lipocalin muteins at a dose of 5 mg/kg or PBS as a vehicle control via local delivery to the lungs from day 0 to day 13 after bleomycin challenge. Animals were also treated with an anti-CTGF monoclonal antibody (SEQ ID NOs: 60 and 61) every other day at a dose of 10 mg/kg via the intravenous route of administration. Animals were sacrificed at day 14 after bleomycin challenge and level of lung fibrosis was analyzed in formalin fixed, paraffin embedded lung tissues by histopathological evaluation using the Ashcroft score (Matsuse modification) and quantitative digital analysis of IHC detected Collagen1a1 protein deposition.

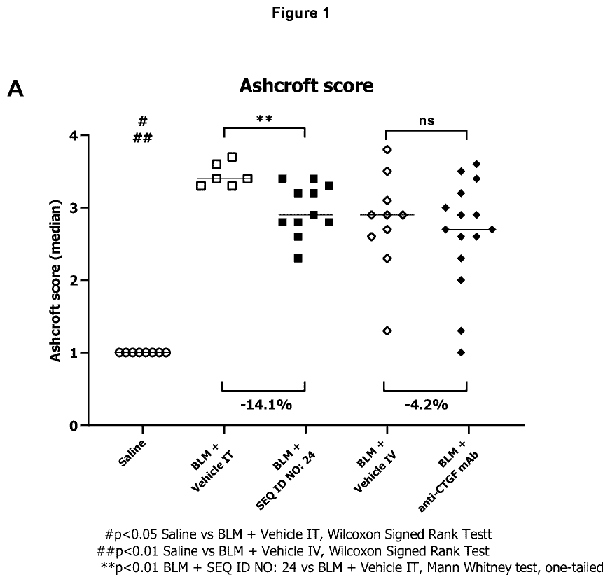

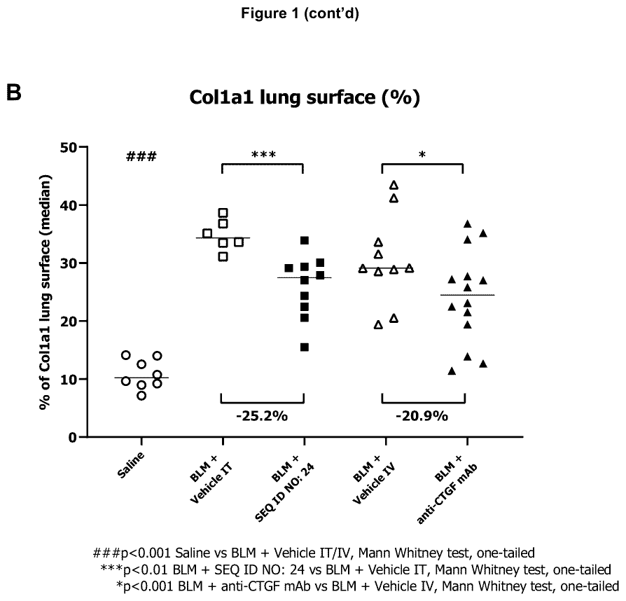

For determination of the Ashcroft score lung tissue slides were stained according to the Crossman's Trichrome method (Gray, Peter (1954) The Microtomist's Formulary and Guide. Blakiston, New York, 1954). The whole pulmonary area subjected for histopathological analysis was evaluated at ten low magnification fields and total score for each animal was calculated as a mean (Ashcroft et al., Journal of clinical pathology 41.4 (1988): 467-470; Matsuse, T., et al. European Respiratory Journal 13.1 (1999): 71-77.). FIG. 1A shows the Ashcroft scores of the different groups analyzed. Bleomycin challenge led to a significant increase in score values of lungs from vehicle control animals when compared to saline challenged, healthy control lungs indicating a strong pro-fibrotic response. Daily, local delivery of the lipocalin mutein of SEQ ID NO: 24 to the lungs of mice significantly decreased the Ashcroft score when compared to PBS-instilled vehicle control lungs. The Ashcroft score was on average reduced by 14.1% when comparing lipocalin-mutein treated lungs to the mean Ashcroft score of vehicle-treated animals with the same route of administration. Of note, treatment with a systemically delivered CTGF-targeting monoclonal antibody (SEQ ID NOs: 60 and 61) had no significant effect on the Ashcroft score and did not attenuate fibrotic lung remodeling.

Collagen1a1 (Col1a1) deposition was analyzed by immunohistochemistry as a second measure for fibrotic lung remodeling. Antigen retrieval of lung tissue sections was performed using an antigen retrieval solution (PT Link modul, DAKO) followed by incubation of tissue slides with a primary rabbit polyclonal anti-COL1A1 antibody for 1 hour (1:2000, LSBio) and detection using the ImmPRESS Detection Kit (Vector, MP-7401). There were two controls performed; Staining without primary antibody and with the rabbit IgG isotype control (Vector) served as control. Stained tissue slides were evaluated for Col1a1 deposition by digital image analysis using the morphometry protocol (CaloPix software, TRIBVN). FIG. 1B indicates the results of the collagen deposition analysis in the different treatment groups as the percentage of the Col1a1 positive lung surface area. Bleomycin challenge strongly induced Col1a1 deposition in the lung tissues when compared to saline challenged lungs. Local lung delivery of the lipocalin mutein of SEQ ID NO: 24 led to a significant reduction of Col1a1 positive lung surface areas when compared to PBS vehicle control treated animals corresponding to a reduction of 25.2% when compared to the mean of respective controls. Systemic delivery of the anti-CTGF monoclonal antibody (SEQ ID NOs: 60 and 61) also significantly reduced Col1a1 levels by a slightly lesser extent of 20.9% over the mean of vehicle control treated animals.

In summary, this experiment showed an overall stronger anti-fibrotic response upon local treatment with the CTGF-targeting lipocalin mutein delivered directly to the lungs when compared to systemic targeting of the protein using a monoclonal antibody. Thus, this study supports our rational for development of inhaled CTGF-targeting lipocalin muteins to achieve a better target engagement and efficacy when compared to inhibitors given via the systemic route.

### Example 12: Effect of CTGF-Targeting Lipocalin Muteins on Lung Organoid Formation

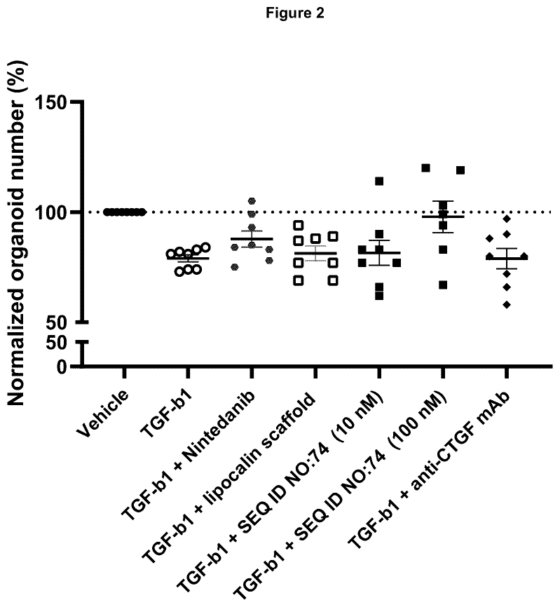

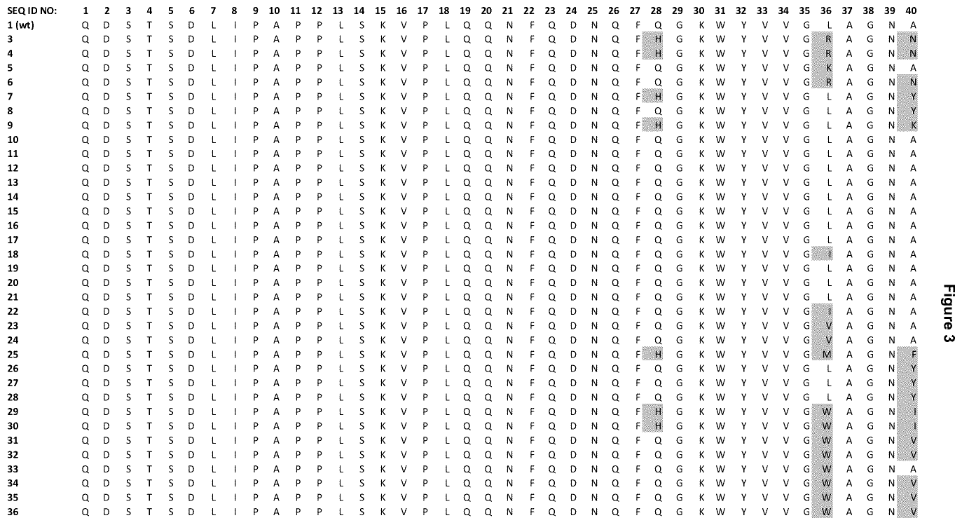

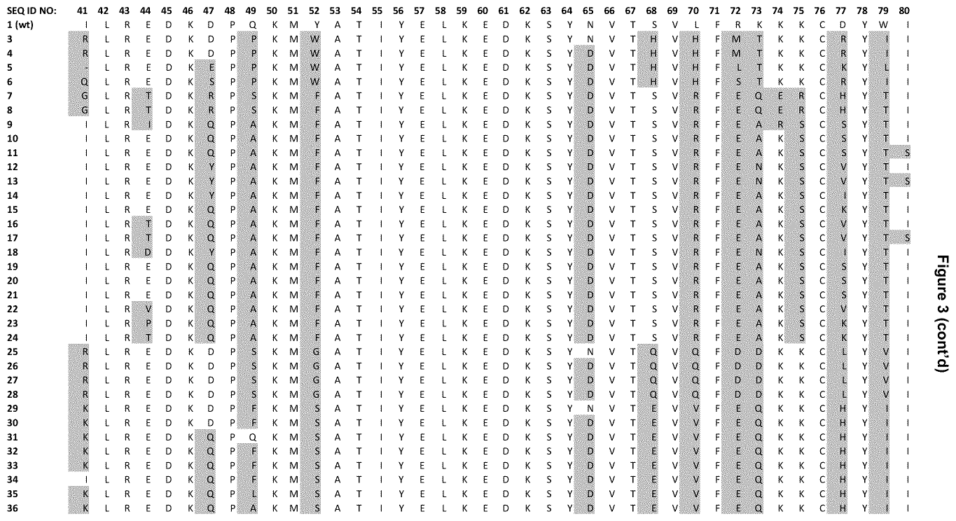

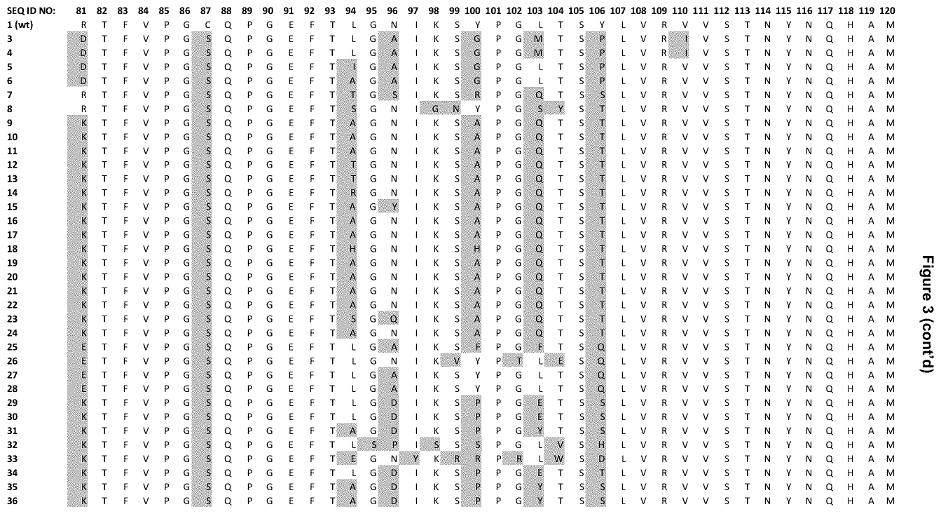

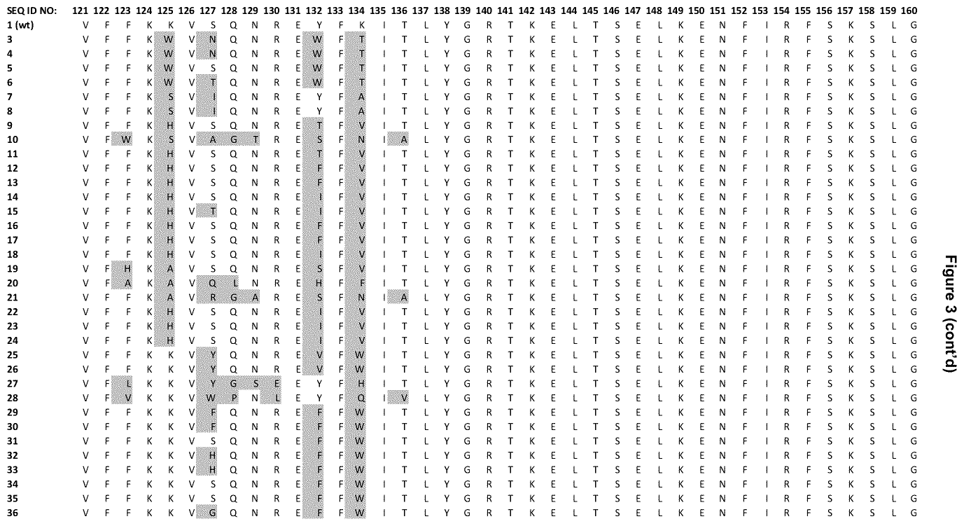

The effect of CTGF-targeting lipocalin muteins was analyzed using an organoid model for lung regeneration. In this model, CCL-206 lung fibroblasts are co-cultured with freshly isolated primary murine Epcam-positive epithelial progenitor cells, and the number of formed organoids is analyzed after 14 days. TGF-b1 pre-treatment of CCL-206 fibroblasts leads to an impaired organoid formation and resembles an impairment of lung regeneration as for example observed in lung disease, such as IPF. The impaired formation of organoids can be rescued by treatment with different drugs, such as nintedanib, which is used as a positive control in this model. In this example, co-cultured cells were treated with nintedanib (100 nM), a lipocalin mutein scaffold control that does not bind to CTGF (100 nM), the CTGF-targeting fusion protein of SEQ ID NO: 74 (10 & 100 nM) and an anti-CTGF monoclonal antibody (SEQ ID NOs: 60 and 61) for 14 days. Results are shown in FIG. 2. Analysis of organoid numbers confirmed the impairment of organoid formation by TGF-b1 stimulation. This effect was partially rescued by treatment with the positive control nintedanib. Of note, treatment with the CTGF-targeting fusion protein of SEQ ID NO: 74 at 100 nM showed an even stronger trend for improved organoid formation, whereas the CTGF-targeting monoclonal antibody (SEQ ID NOs: 60 and 61) had no effect on organoid formation.

### Example 13: Binding to Activated Human Lung Fibroblasts

Binding of the CTGF-targeting lipocalin muteins and fusion proteins disclosed herein to CTGF-expressing, TGF-β1-stimulated normal human lung fibroblasts (NHLFs) was tested by incubating NHLFs for 24 h with constructs and 10 ng/ml TGF-β1, which induces CTGF expression. NHLFs without TGF-β1 stimulation were included as negative control. Constructs were detected with a secondary antibody against the lipocalin scaffold coupled to AlexaFluor647 after NHLFs were fixed in 4% PFA and blocked with 5% BSA. Images were acquired on the Cytation5 Reader (Biotek). Signals were quantified in Gene5 software and normalized to the respective controls. For the experiments, cells were grown under conditions that facilitate pseudo-3D extracellular matrix deposition as described by Good et al. (Good et al., BMC Biomed Eng, 1:14 (2019)).

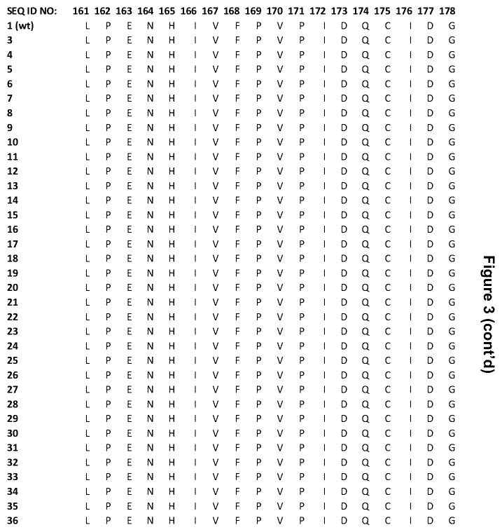

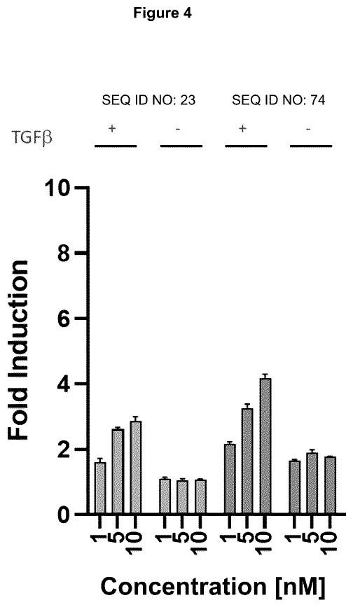

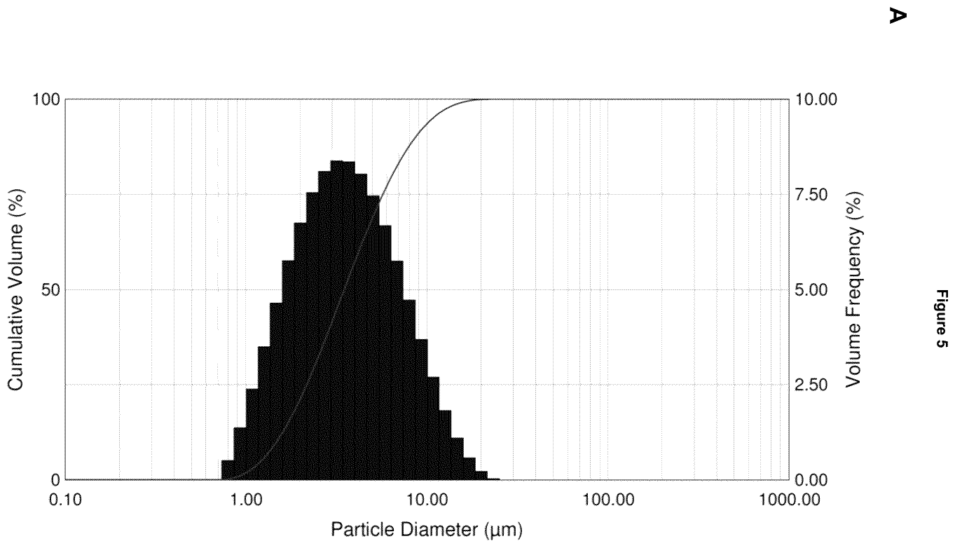

Representative data are shown in FIG. 4 and illustrate the ability of the CTGF-targeting lipocalin muteins and fusion proteins disclosed herein to bind to activated human lung fibroblasts expressing CTGF in a concentration-dependent manner.

### Example 14: Nebulization Behavior

The suitability of the CTGF-targeting lipocalin muteins and fusion proteins disclosed herein for administration via inhalation was tested using a commercially available vibrating mesh nebulizer (Philips InnoSpire Go®). Droplet size distribution was characterized by laser diffraction using the nebulizer in conjunction with Malvern Spraytec and inhalation cell.

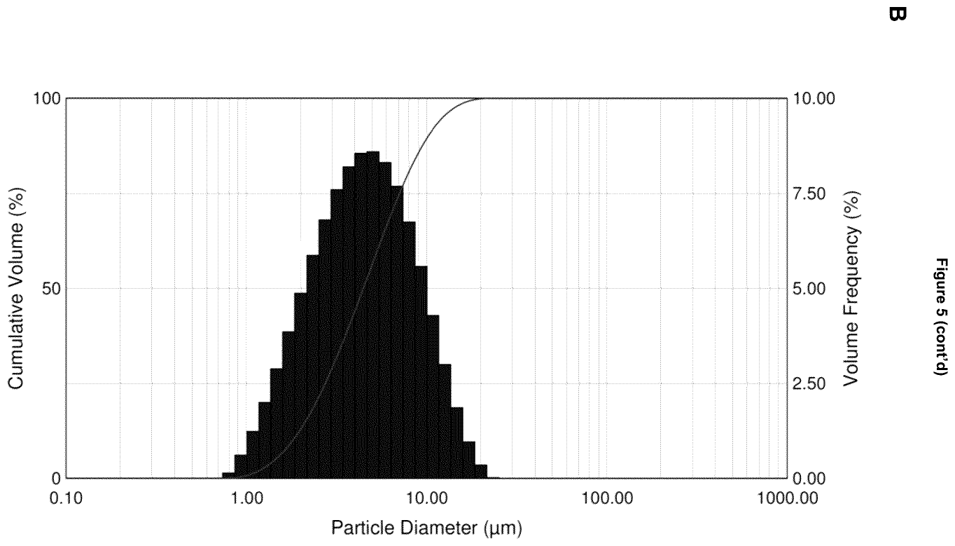

Representative data for the exemplary CTGF-targeting lipocalin mutein of SEQ ID NO: 23 (A) and for the fusion protein of SEQ ID NO: 74 (B) are shown in FIG. 5 and below Table 10. These data illustrate that the biophysical properties of the CTGF-targeting lipocalin muteins and fusion proteins disclosed herein allow the generation of aerosols that are appropriate for inhalative applications in humans and that are characterized by particles small enough to reach effective deposition in the lung. Nebulization of the lipocalin muteins and fusion proteins had no negative impact on the stability and activity of the molecules (data not shown).

TABLE 10
Droplet size distribution upon nebulization.

| SEQ | | | | % V < | % V < | % V < |
|---|---|---|---|---|---|---|
| ID NO: | Dv10(μm) | Dv50(μm) | Dv90(μm) | 10 μm | 5 μm | 1 μm |
| 23 | 1.471 | 3.484 | 8.622 | 93.55 | 68.97 | 1.874 |
| 74 | 1.824 | 4.589 | 10.5 | 88.4 | 54.81 | 0.984 |

### Example 15: Lung Tissue Distribution in Fibrotic Lungs of Mice

Lung tissue biodistribution of fluorescently labeled muteins and fusion proteins was investigated in fibrotic lungs of mice. At day 21 after bleomycin challenge (see Example 11), mice were either treated with the Alexa-647-labeled exemplary lipocalin mutein of SEQ ID NO: 23, the Alexa-647-labeled exemplary fusion protein of SEQ ID NO: 74 (both administered to the lung) or with an Alexa-647-labeled CTGF-targeting monoclonal antibody (SEQ ID NOs: 60 and 61) delivered systemically via intravenous infusion. Lung tissue biodistribution of differentially administered compounds was analyzed by Light Sheet Microscopy imaging of the left lung at 2, 8 and 24 hours after delivery.

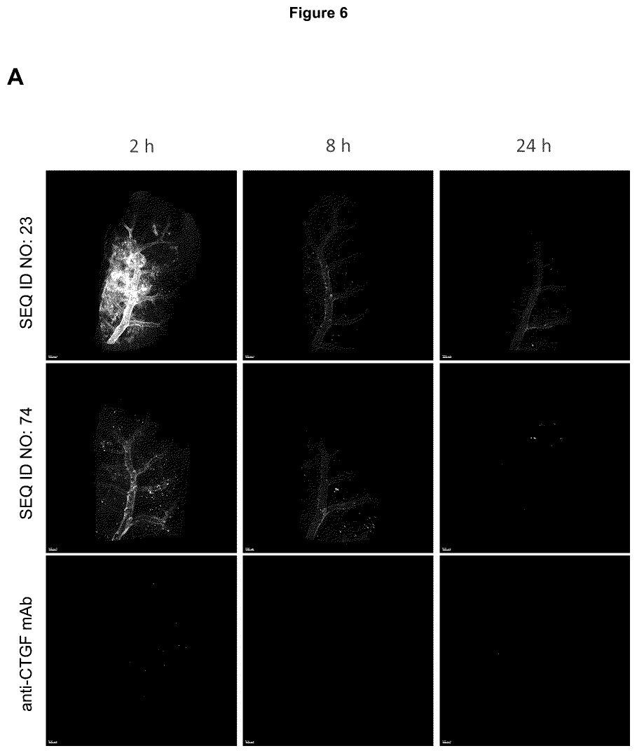

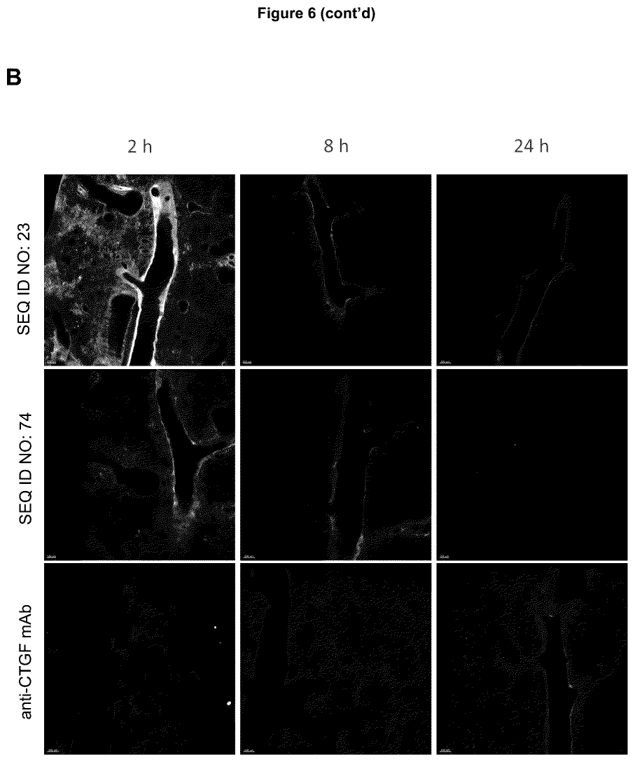

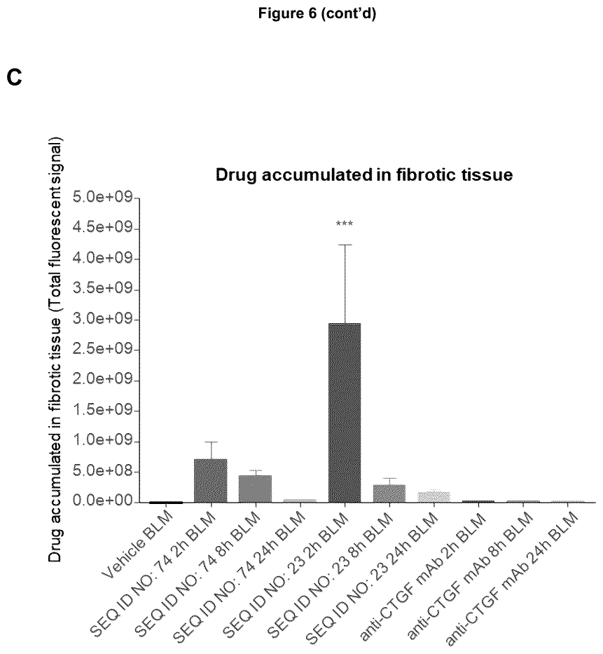

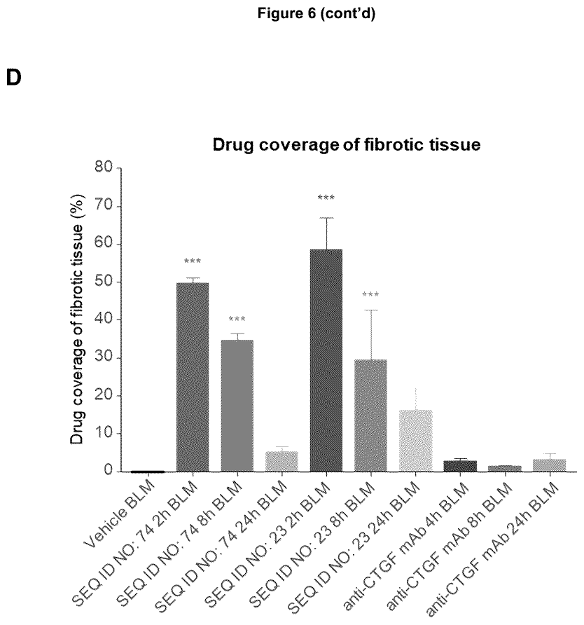

Representative 3D overview images are shown in FIG. 6A, and representative magnified 2D sections from 3D scanned lungs are shown in FIG. 6B. FIG. 6C and FIG. 6D show the total compound fluorescence in fibrotic areas and the volume fraction of the fibrotic area targeted by the compounds, respectively. These data clearly show that CTGF-targeting lipocalin muteins as well as (to a lesser, but still substantial degree) fusion proteins comprising such muteins allow to effectively target fibrotic tissue in the lung of mice, including distal areas of the lung. Such effective targeting could not be achieved with a systemically administered anti-CTGF monoclonal antibody.

### Example 16: Mouse Lung PK

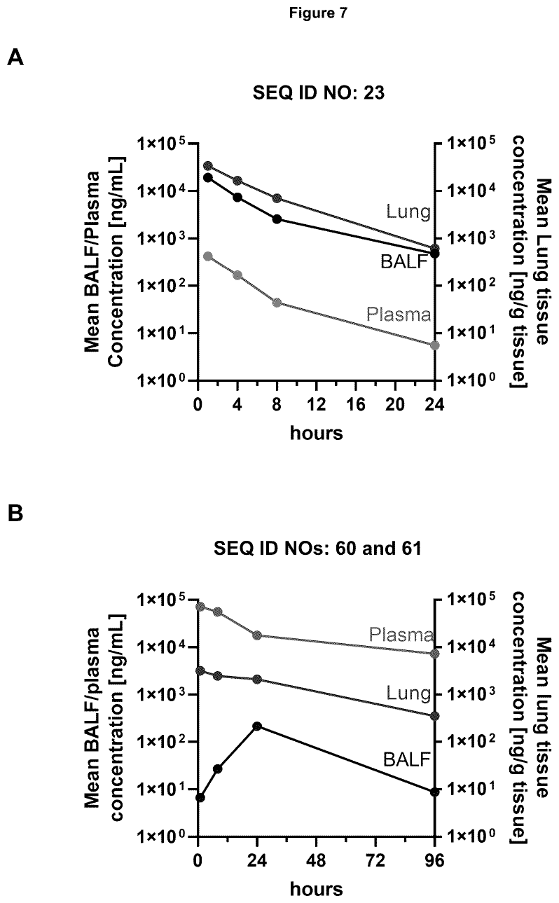

The pharmacokinetic (PK) profiles of the exemplary lipocalin mutein of SEQ ID NO: 23 and the anti-CTGF monoclonal antibody of SEQ ID NOs: 60 and 61 were analyzed in bronchoalveolar lavage fluid (BALF), lung tissue and plasma of mice. The lipocalin mutein (100 μg/mouse) was oropharyngeally administered to mice, and exposure in different compartments was measured after 2, 4, 8 and 24 h by ELISA (FIG. 7A). 100 μg of the antibody were administered to mice via intravenous infusion, and exposure was measured after 1, 8, 24 and 96 h by ELISA (FIG. 7B).

As shown in FIG. 7A, PK analysis of the oropharyngeally delivered lipocalin mutein of SEQ ID NO: 23 confirmed significant exposure in the lung over 24 h supporting once daily pulmonary delivery. While the lipocalin mutein achieved high exposure in the lung with only approx. 1% reaching the plasma, pulmonary exposure of the systemically delivered antibody was significantly lower in BALF and lung tissue with only approx. 20% reaching the lung (Table 11).

TABLE 11
Lung and plasma exposure.

| | Lung exposure compared | Plasma exposure |
|---|---|---|
| SEQ ID NO: | to plasma (%) | compared to lung (%) |
| 23 | 100* | 0.9 |
| 60 and 61 | 20.3 | 100* |

*Set to 100% for compartment of administration

Similar results were obtained with a fusion protein comprising two lipocalin proteins, wherein the fusion protein exhibited increased retention time in BALF and in the lung as compared to a single lipocalin protein (data not shown).

Embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present embodiments have been specifically disclosed by preferred embodiments and optional features, modification and variations thereof may be resorted to by those skilled in the art, and that such modifications and variations are considered to be within the scope of this invention. All patents, patent applications, textbooks and peer-reviewed publications described herein are hereby incorporated by reference in their entirety. Furthermore, where a definition or use of a term in a reference, which is incorporated by reference herein is inconsistent or contrary to the definition of that term provided herein, the definition of that term provided herein applies and the definition of that term in the reference does not apply. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features are described in terms of Markush groups, those skilled in the art will recognize that the disclosure is also thereby described in terms of any individual member or subgroup of members of the Markush group. Further embodiments will become apparent from the following claims.

Equivalents: Those skilled in the art will recognize or be able to ascertain using no more than routine experimentation many equivalents to the specific embodiments of the invention described herein. Such equivalents are intended to be encompassed by the following claims. All publications, patents and patent applications mentioned in this specification are herein incorporated by reference into the specification to the same extent as if each individual publication, patent or patent application was specifically and individually indicated to be incorporated herein by reference.

1. A lipocalin mutein that is capable of binding CTGF with a detectable affinity.

2. The lipocalin mutein of claim 1, wherein the mutein is capable of binding CTGF with an affinity measured by a K_{D} of about 500 nM or lower, about 400 nM or lower, about 300 nM or lower, about 200 nM or lower, about 150 nM or lower, about 100 nM or lower, about 50 nM or lower, or about 30 nM or lower.

3. The lipocalin mutein of claim 1 or 2, wherein the mutein binds CTGF with an EC₅₀ value of about 250 nM or lower, about 200 nM or lower, about 150 nM or lower, about 100 nM or lower, or about 50 nM or lower.

4. The lipocalin mutein of any one of claims 1-3, wherein the mutein is cross-reactive with cynomolgus CTGF.

5. The lipocalin mutein of any one of claims 1-4, wherein the mutein is cross-reactive with murine CTGF.

6. The lipocalin mutein of any one of claims 1-5, wherein the mutein is cross-reactive with rat CTGF.

7. The lipocalin mutein of any one of claims 1-6, wherein the mutein competes with an antibody having the heavy chain and light chain sequences of SEQ ID NOs: 60 and 61 for the binding of CTGF.

8. The lipocalin mutein of any one of claims 1-6, wherein the mutein does not compete with an antibody having the heavy chain and light chain sequences of SEQ ID NOs: 60 and 61 for the binding of CTGF.

9. The lipocalin mutein of any one of claims 1-8, wherein the mutein is not cross-reactive with one or more members of the CCN protein family selected from the group consisting of CYR61 (CCN1), NOV (CCN3), WISP-1 (CCN4), WISP-2 (CCN5), and WISP-3 (CCN6).

10. The lipocalin mutein of any one of claims 1-9, wherein the mutein is capable of providing an anti-fibrotic effect in vivo.

11. The lipocalin mutein of any one of claims 1-10, wherein the mutein comprises at one or more positions corresponding to positions 28, 36, 40, 41, 44, 47, 49, 52, 65, 68, 70, 72, 73, 74, 75, 77, 79, 80, 81, 87, 94, 95, 96, 97, 98, 99, 100, 102, 103, 104, 106, 110, 123, 125, 127, 128, 129, 130, 132, 134, and 136 of the linear polypeptide sequence of mature hNGAL (SEQ ID NO: 1), ten or more of the following mutated amino acid residues: Gln 28→His; Leu 36→Arg, Lys, Ile, Val, Met, or Trp; Ala 40→Asn, Tyr, Lys, Phe, Ile, or Val; Ile 41→Arg, deletion of Ile 41, Gln, Gly, or Lys; Glu 44→Thr, Ile, Asp, Val, or Pro; Asp 47→Glu, Ser, Arg, Gln, or Tyr; Gln 49→Pro, Ser, Ala, Phe, Leu, or Ala; Tyr 52→Trp, Phe, Gly, or Ser; Asn 65→Asp; Ser 68→His, Gln, or Glu; Leu 70→His, Arg, Gln, or Val; Arg 72→Met, Leu, Ser, Glu, or Asp; Lys 73→Thr, Gln, Ala, Asn, or Asp; Lys 74→Glu or Arg; Lys 75→Arg or Ser; Asp 77→Arg, Lys, His, Ser, Val, Ile, or Leu; Trp 79→Ile, Leu, Thr, or Val; Ile 80→Ser; Arg 81→Asp, Lys, or Glu; Cys 87→Ser; Leu 94→Ile, Ala, Thr, Ser, Arg, His, or Glu; Gly 95→Ser; Asn 96→Ala, Ser, Tyr, Gln, Asp, or Pro; Ile 97→Tyr; Lys 98→Gly or Ser; Ser 99→Asn, Val, or Arg; Tyr 100→Gly, Arg, Ala, His, Phe, Pro, or Ser; Gly 102→Thr or Arg; Leu 103→Met, Gln, Ser, Phe, Glu, or Tyr; Thr 104→Tyr, Glu, Val, or Trp; Tyr 106→Pro, Ser, Thr, Gln, His, or Asp; Val 110→Ile; Phe 123→Trp, His, Ala, Leu, or Val; Lys 125→Trp, Ser, His, or Ala; Ser 127→Asn, Thr, Ile, Ala, Gln, Arg, Tyr, Trp, Phe, His, or Gly; Gln 128→Gly, Leu, or Pro; Asn 129→Thr, Ala, or Ser; Arg 130→Glu or Leu; Tyr 132→Trp, Thr, Ser, Phe, Ile, His, or Val; Lys 134→Thr, Ala, Val, Asn, Phe, Trp, His, or Gln; and Thr 136→Ala or Val.

12. The lipocalin mutein of any one of claims 1-11, wherein the mutein comprises one of the following sets of mutated amino acid residues in comparison with the linear polypeptide sequence of mature hNGAL (SEQ ID NO: 1):
(a) Leu 36→Arg, Ala 40→Asn, Ile 41→Arg, Gln 49→Pro, Tyr 52→Trp, Ser 68→His, Leu 70→His, Arg 72→Met, Lys 73→Thr, Asp 77→Arg, Trp 79→Ile, Arg 81→Asp, Asn 96→Ala, Tyr 100→Gly, Leu 103→Met, Tyr 106→Pro, Val 110→Ile, Lys 125→Trp, Ser 127→Asn, Tyr 132→Trp, and Lys 134→Thr;
(b) Leu 36→Arg, Ala 40→Asn, Ile 41→Arg, Gln 49→Pro, Tyr 52→Trp, Ser 68→His, Leu 70→His, Arg 72→Met, Lys 73→Thr, Asp 77→Arg, Trp 79→Ile, Arg 81→Asp, Asn 96→Ala, Tyr 100→Gly, Leu 103→Met, Tyr 106→Pro, Val 110→Ile, Lys 125→Trp, Ser 127→Asn, Tyr 132→Trp, and Lys 134→Thr;
(c) Leu 36→Lys, Ile 41→deletion of Ile 41, Asp 47→Glu, Gln 49→Pro, Tyr 52→Trp, Ser 68→His, Leu 70→His, Arg 72→Leu, Lys 73→Thr, Asp 77→Lys, Trp 79→Leu, Arg 81→Asp, Leu 94→Ile, Asn 96→Ala, Tyr 100→Gly, Tyr 106→Pro, Lys 125→Trp, Tyr 132→Trp, and Lys 134→Thr;
(d) Leu 36→Arg, Ala 40→Asn, Ile 41→Gln, Asp 47→Ser, Gln 49→Pro, Tyr 52→Trp, Ser 68→His, Leu 70→His, Arg 72→Ser, Lys 73→Thr, Asp 77→Arg, Trp 79→Ile, Arg 81→Asp, Leu 94→Ala, Asn 96→Ala, Tyr 100→Gly, Tyr 106→Pro, Lys 125→Trp, Ser 127→Thr, Tyr 132→Trp, and Lys 134→Thr;
(e) Ala 40→Tyr, Ile 41→Gly, Glu 44→Thr, Asp 47→Arg, Gln 49→Ser, Tyr 52→Phe, Leu 70→Arg, Arg 72→Glu, Lys 73→Gln, Lys 74→Glu, Lys 75→Arg, Asp 77→His, Trp 79→Thr, Leu 94→Thr, Asn 96→Ser, Tyr 100→Arg, Leu 103→Gln, Tyr 106→Ser, Lys 125→Ser, Ser 127→Ile, and Lys 134→Ala;
(f) Ala 40→Tyr, Ile 41→Gly, Glu 44→Thr, Asp 47→Arg, Gln 49→Ser, Tyr 52→Phe, Leu 70→Arg, Arg 72→Glu, Lys 73→Gln, Lys 74→Glu, Lys 75→Arg, Asp 77→His, Trp 79→Thr, Leu 94→Ser, Lys 98→Gly, Ser 99→Asn, Leu 103→Ser, Thr 104→Tyr, Tyr 106→Thr, Lys 125→Ser, Ser 127→Ile, and Lys 134→Ala;
(g) Ala 40→Lys, Glu 44→Ile, Asp 47→Gln, Gln 49→Ala, Tyr 52→Phe, Leu 70→Arg, Arg 72→Glu, Lys 73→Ala, Lys 74→Arg, Lys 75→Ser, Asp 77→Ser, Trp 79→Thr, Arg 81→Lys, Leu 94→Ala, Tyr 100→Ala, Leu 103→Gln, Tyr 106→Thr, Lys 125→His, Tyr 132→Thr, and Lys 134→Val;
(h) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Arg 81→Lys, Leu 94→Ala, Tyr 100→Ala, Leu 103→Gln, Tyr 106→Thr, Phe 123→Trp, Lys 125→Ser, Ser 127→Ala, Gln 128→Gly, Asn 129→Thr, Tyr 132→Ser, and Lys 134→Asn, Thr 136→Ala;
(i) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Ile 80→Ser, Arg 81→Lys, Leu 94→Ala, Tyr 100→Ala, Leu 103→Gln, Tyr 106→Thr, Lys 125→His, Tyr 132→Thr, and Lys 134→Val;
(j) Asp 47→Tyr, Gln 49→Ala, Tyr 52→Phe, Leu 70→Arg, Arg 72→Glu, Lys 73→Asn, Lys 75→Ser, Asp 77→Val, Trp 79→Thr, Arg 81→Lys, Leu 94→Thr, Tyr 100→Ala, Leu 103→Gln, Tyr 106→Thr, Lys 125→His, Tyr 132→Phe, and Lys 134→Val;
(k) Asp 47→Tyr, Gln 49→Ala, Tyr 52→Phe, Leu 70→Arg, Arg 72→Glu, Lys 73→Asn, Lys 75→Ser, Asp 77→Val, Trp 79→Thr, Ile 80→Ser, Arg 81→Lys, Leu 94→Thr, Tyr 100→Ala, Leu 103→Gln, Tyr 106→Thr, Lys 125→His, Tyr 132→Phe, and Lys 134→Val;
(l) Asp 47→Tyr, Gln 49→Ala, Tyr 52→Phe, Leu 70→Arg, Arg 72→Glu, Lys 73→Ala, Lys 75→Ser, Asp 77→Ile, Trp 79→Thr, Arg 81→Lys, Leu 94→Arg, Tyr 100→Ala, Leu 103→Gln, Tyr 106→Thr, Lys 125→His, Tyr 132→Ile, and Lys 134→Val;
(m) Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val;
(n) Glu 44→Thr, Asp 47→Gln, Gln 49→Ala, Tyr 52→Phe, Leu 70→Arg, Arg 72→Glu, Lys 73→Ala, Lys 75→Ser, Asp 77→Val, Trp 79→Thr, Arg 81→Lys, Leu 94→Ala, Tyr 100→Ala, Leu 103→Gln, Tyr 106→Thr, Lys 125→His, Tyr 132→Phe, and Lys 134→Val;
(o) Glu 44→Thr, Asp 47→Gln, Gln 49→Ala, Tyr 52→Phe, Leu 70→Arg, Arg 72→Glu, Lys 73→Ala, Lys 75→Ser, Asp 77→Val, Trp 79→Thr, Ile 80→Ser, Arg 81→Lys, Leu 94→Ala, Tyr 100→Ala, Leu 103→Gln, Tyr 106→Thr, Lys 125→His, Tyr 132→Phe, and Lys 134→Val;
(p) Leu 36→Ile, Glu 44→Asp, Asp 47→Tyr, Gln 49→Ala, Tyr 52→Phe, Leu 70→Arg, Arg 72→Glu, Lys 73→Asn, Lys 75→Ser, Asp 77→Ile, Trp 79→Thr, Arg 81→Lys, Leu 94→His, Tyr 100→His, Leu 103→Gln, Tyr 106→Thr, Lys 125→His, Tyr 132→Ile, and Lys 134→Val;
(q) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Arg 81→Lys, Leu 94→Ala, Tyr 100→Ala, Leu 103→Gln, Tyr 106→Thr, Phe 123→His, Lys 125→Ala, Tyr 132→Ser, and Lys 134→Val;
(r) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Arg 81→Lys, Leu 94→Ala, Tyr 100→Ala, Leu 103→Gln, Tyr 106→Thr, Phe 123→Ala, Lys 125→Ala, Ser 127→Gln, Gln 128→Leu, Tyr 132→His, and Lys 134→Phe;
(s) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Arg 81→Lys, Leu 94→Ala, Tyr 100→Ala, Leu 103→Gln, Tyr 106→Thr, Lys 125→Ala, Ser 127→Arg, Gln 128→Gly, Asn 129→Ala, Tyr 132→Ser, and Lys 134→Asn, Thr 136→Ala;
(t) Leu 36→Ile, Glu 44→Val, Asp 47→Gln, Gln 49→Ala, Tyr 52→Phe, Leu 70→Arg, Arg 72→Glu, Lys 73→Ala, Lys 75→Ser, Asp 77→Val, Trp 79→Thr, Arg 81→Lys, Leu 94→Ala, Tyr 100→Ala, Leu 103→Gln, Tyr 106→Thr, Lys 125→His, Tyr 132→Ile, and Lys 134→Val;
(u) Leu 36→Val, Glu 44→Pro, Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ser, Asn 96→Gln, Tyr 100→Ala, Leu 103→Gln, Tyr 106→Thr, Lys 125→His, Tyr 132→Ile, and Lys 134→Val;
(v) Leu 36→Val, Glu 44→Thr, Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Tyr 100→Ala, Leu 103→Gln, Tyr 106→Thr, Lys 125→His, Tyr 132→Ile, and Lys 134→Val;
(w) Leu 36→Met, Ala 40→Phe, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 100→Phe, Leu 103→Phe, Tyr 106→Gln, Ser 127→Tyr, Tyr 132→Val, and Lys 134→Trp;
(x) Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Ser 99→Val, Gly 102→Thr, Thr 104→Glu, Tyr 106→Gln, Ser 127→Tyr, Tyr 132→Val, and Lys 134→Trp;
(y) Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 106→Gln, Phe 123→Leu, Ser 127→Tyr, Gln 128→Gly, Asn 129→Ser, Arg 130→Glu, and Lys 134→His;
(z) Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 106→Gln, Phe 123→Val, Ser 127→Trp, Gln 128→Pro, Arg 130→Leu, and Lys 134→Gln, Thr 136→Val;
(aa) Leu 36→Trp, Ala 40→Ile, Ile 41→Lys, Gln 49→Phe, Tyr 52→Ser, Ser 68→Glu, Leu 70→Val, Arg 72→Glu, Lys 73→Gln, Asp 77→His, Trp 79→Ile, Arg 81→Lys, Asn 96→Asp, Tyr 100→Pro, Leu 103→Glu, Tyr 106→Ser, Ser 127→Phe, Tyr 132→Phe, and Lys 134→Trp;
(bb) Leu 36→Trp, Ala 40→Ile, Ile 41→Lys, Gln 49→Phe, Tyr 52→Ser, Ser 68→Glu, Leu 70→Val, Arg 72→Glu, Lys 73→Gln, Asp 77→His, Trp 79→Ile, Arg 81→Lys, Asn 96→Asp, Tyr 100→Pro, Leu 103→Glu, Tyr 106→Ser, Ser 127→Phe, Tyr 132→Phe, and Lys 134→Trp;
(cc) Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp;
(dd) Leu 36→Trp, Ala 40→Val, Ile 41→Lys, Asp 47→Gln, Gln 49→Phe, Tyr 52→Ser, Ser 68→Glu, Leu 70→Val, Arg 72→Glu, Lys 73→Gln, Asp 77→His, Trp 79→Ile, Arg 81→Lys, Gly 95→Ser, Asn 96→Pro, Lys 98→Ser, Tyr 100→Ser, Thr 104→Val, Tyr 106→His, Ser 127→His, Tyr 132→Phe, and Lys 134→Trp;
(ee) Leu 36→Trp, Ile 41→Lys, Asp 47→Gln, Gln 49→Phe, Tyr 52→Ser, Ser 68→Glu, Leu 70→Val, Arg 72→Glu, Lys 73→Gln, Asp 77→His, Trp 79→Ile, Arg 81→Lys, Leu 94→Glu, Ile 97→Tyr, Ser 99→Arg, Tyr 100→Arg, Gly 102→Arg, Thr 104→Trp, Tyr 106→Asp, Ser 127→His, Tyr 132→Phe, and Lys 134→Trp;
(ff) Leu 36→Trp, Ala 40→Val, Asp 47→Gln, Gln 49→Phe, Tyr 52→Ser, Ser 68→Glu, Leu 70→Val, Arg 72→Glu, Lys 73→Gln, Asp 77→His, Trp 79→Ile, Arg 81→Lys, Asn 96→Asp, Tyr 100→Pro, Leu 103→Glu, Tyr 106→Thr, Tyr 132→Phe, and Lys 134→Trp;
(gg) Leu 36→Trp, Ala 40→Val, Ile 41→Lys, Asp 47→Gln, Gln 49→Leu, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp; or
(hh) Leu 36→Trp, Ala 40→Val, Ile 41→Lys, Asp 47→Gln, Gln 49→Ala, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Ser 127→Gly, Tyr 132→Phe, and Lys 134→Trp.

13. The lipocalin mutein of any one of claims 1-12, wherein the mutein binds to an epitope on CTGF that overlaps with the epitope of an antibody having the heavy chain and light chain sequences of SEQ ID NOs: 60 and 61.

14. The lipocalin mutein of any one of claims 1-13, wherein the mutein has at least 85%, at least 90%, at least 95%, at least 98%, or at least 99% sequence identity to an amino acid sequence selected from the group consisting of SEQ ID NOs: 3-36.

15. The lipocalin mutein of any one of claims 1-14, wherein the mutein comprises an amino acid sequence selected from the group consisting of SEQ ID NOs: 3-36 or a fragment or variant thereof.

16. The lipocalin mutein of any one of claims 1-15, wherein the mutein is conjugated to a compound selected from the group consisting of an organic molecule, an enzyme label, a radioactive label, a colored label, a fluorescent label, a chromogenic label, a luminescent label, a hapten, digoxigenin, biotin, a cytostatic agent, a toxin, a metal complex, a metal, and colloidal gold.

17. The lipocalin mutein of any one of claims 1-16, wherein the mutein is fused at its N-terminus and/or its C-terminus to a fusion partner that is a protein, a protein domain, a peptide, or a lipocalin mutein.

18. The lipocalin mutein of any one of claims 1-17, wherein the mutein is fused at its N-terminus and/or its C-terminus to a fusion partner that is an antibody or antibody fragment.

19. The lipocalin mutein of any one of claims 1-18, wherein the mutein is conjugated to a compound that extends the serum half-life of the mutein.

20. The lipocalin mutein of claim 19, wherein the compound that extends the serum half-life is selected from the group consisting of a polyethylene glycol (PEG) molecule, hydroxyethyl starch, an Fc part of an immunoglobulin, a C_{H}3 domain of an immunoglobulin, a C_{H}4 domain of an immunoglobulin, an albumin binding peptide, and an albumin binding protein.

21. A fusion protein comprising two lipocalin muteins of any one of claims 1-20.

22. The fusion protein of claim 21, wherein the fusion protein comprises two lipocalin muteins that comprise the following sets of mutated amino acid residues in comparison with the linear polypeptide sequence of mature hNGAL (SEQ ID NO: 1), respectively:
(a) Leu 36→Arg, Ala 40→Asn, Ile 41→Gln, Asp 47→Ser, Gln 49→Pro, Tyr 52→Trp, Ser 68→His, Leu 70→His, Arg 72→Ser, Lys 73→Thr, Asp 77→Arg, Trp 79→Ile, Arg 81→Asp, Leu 94→Ala, Asn 96→Ala, Tyr 100→Gly, Tyr 106→Pro, Lys 125→Trp, Ser 127→Thr, Tyr 132→Trp, and Lys 134→Thr; and
Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp;
(b) Leu 36→Arg, Ala 40→Asn, Ile 41→Gln, Asp 47→Ser, Gln 49→Pro, Tyr 52→Trp, Ser 68→His, Leu 70→His, Arg 72→Ser, Lys 73→Thr, Asp 77→Arg, Trp 79→Ile, Arg 81→Asp, Leu 94→Ala, Asn 96→Ala, Tyr 100→Gly, Tyr 106→Pro, Lys 125→Trp, Ser 127→Thr, Tyr 132→Trp, and Lys 134→Thr; and
Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val;
(c) Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 106→Gln, Phe 123→Val, Ser 127→Trp, Gln 128→Pro, Arg 130→Leu, and Lys 134→Gln, Thr 136→Val; and
Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val;
(d) Leu 36→Arg, Ala 40→Asn, Ile 41→Arg, Gln 49→Pro, Tyr 52→Trp, Ser 68→His, Leu 70→His, Arg 72→Met, Lys 73→Thr, Asp 77→Arg, Trp 79→Ile, Arg 81→Asp, Asn 96→Ala, Tyr 100→Gly, Leu 103→Met, Tyr 106→Pro, Val 110→Ile, Lys 125→Trp, Ser 127→Asn, Tyr 132→Trp, and Lys 134→Thr; and
Ala 40→Lys, Glu 44→Ile, Asp 47→Gln, Gln 49→Ala, Tyr 52→Phe, Leu 70→Arg, Arg 72→Glu, Lys 73→Ala, Lys 74→Arg, Lys 75→Ser, Asp 77→Ser, Trp 79→Thr, Arg 81→Lys, Leu 94→Ala, Tyr 100→Ala, Leu 103→Gln, Tyr 106→Thr, Lys 125→His, Tyr 132→Thr, and Lys 134→Val;
(e) Ala 40→Lys, Glu 44→Ile, Asp 47→Gln, Gln 49→Ala, Tyr 52→Phe, Leu 70→Arg, Arg 72→Glu, Lys 73→Ala, Lys 74→Arg, Lys 75→Ser, Asp 77→Ser, Trp 79→Thr, Arg 81→Lys, Leu 94→Ala, Tyr 100→Ala, Leu 103→Gln, Tyr 106→Thr, Lys 125→His, Tyr 132→Thr, and Lys 134→Val; and
Leu 36→Arg, Ala 40→Asn, Ile 41→Arg, Gln 49→Pro, Tyr 52→Trp, Ser 68→His, Leu 70→His, Arg 72→Met, Lys 73→Thr, Asp 77→Arg, Trp 79→Ile, Arg 81→Asp, Asn 96→Ala, Tyr 100→Gly, Leu 103→Met, Tyr 106→Pro, Val 110→Ile, Lys 125→Trp, Ser 127→Asn, Tyr 132→Trp, and Lys 134→Thr;
(f) Ala 40→Lys, Glu 44→Ile, Asp 47→Gln, Gln 49→Ala, Tyr 52→Phe, Leu 70→Arg, Arg 72→Glu, Lys 73→Ala, Lys 74→Arg, Lys 75→Ser, Asp 77→Ser, Trp 79→Thr, Arg 81→Lys, Leu 94→Ala, Tyr 100→Ala, Leu 103→Gln, Tyr 106→Thr, Lys 125→His, Tyr 132→Thr, and Lys 134→Val; and
Leu 36→Trp, Ala 40→Ile, Ile 41→Lys, Gln 49→Phe, Tyr 52→Ser, Ser 68→Glu, Leu 70→Val, Arg 72→Glu, Lys 73→Gln, Asp 77→His, Trp 79→Ile, Arg 81→Lys, Asn 96→Asp, Tyr 100→Pro, Leu 103→Glu, Tyr 106→Ser, Ser 127→Phe, Tyr 132→Phe, and Lys 134→Trp;
(g) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Ile 80→Ser, Arg 81→Lys, Leu 94→Ala, Tyr 100→Ala, Leu 103→Gln, Tyr 106→Thr, Lys 125→His, Tyr 132→Thr, and Lys 134→Val; and
Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp;
(h) Asp 47→Gln, Gln 49→Ala, Tyr 52→Phe, Leu 70→Arg, Arg 72→Glu, Lys 73→Ala, Lys 75→Ser, Asp 77→Ser, Trp 79→Thr, Ile 80→Ser, Arg 81→Lys, Leu 94→Ala, Tyr 100→Ala, Leu 103→Gln, Tyr 106→Thr, Lys 125→His, Tyr 132→Thr, and Lys 134→Val; and
Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 106→Gln, Phe 123→Val, Ser 127→Trp, Gln 128→Pro, Arg 130→Leu, and Lys 134→Gln, Thr 136→Val;
(i) Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp; and
Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val;
(j) Asp 47→Tyr, Gln 49→Ala, Tyr 52→Phe, Leu 70→Arg, Arg 72→Glu, Lys 73→Asn, Lys 75→Ser, Asp 77→Val, Trp 79→Thr, Arg 81→Lys, Leu 94→Thr, Tyr 100→Ala, Leu 103→Gln, Tyr 106→Thr, Lys 125→His, Tyr 132→Phe, and Lys 134→Val; and
Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp;
(k) Asp 47→Tyr, Gln 49→Ala, Tyr 52→Phe, Leu 70→Arg, Arg 72→Glu, Lys 73→Asn, Lys 75→Ser, Asp 77→Val, Trp 79→Thr, Arg 81→Lys, Leu 94→Thr, Tyr 100→Ala, Leu 103→Gln, Tyr 106→Thr, Lys 125→His, Tyr 132→Phe, and Lys 134→Val; and
Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 106→Gln, Phe 123→Val, Ser 127→Trp, Gln 128→Pro, Arg 130→Leu, and Lys 134→Gln, Thr 136→Val;
(l) Asp 47→Tyr, Gln 49→Ala, Tyr 52→Phe, Leu 70→Arg, Arg 72→Glu, Lys 73→Asn, Lys 75→Ser, Asp 77→Val, Trp 79→Thr, Ile 80→Ser, Arg 81→Lys, Leu 94→Thr, Tyr 100→Ala, Leu 103→Gln, Tyr 106→Thr, Lys 125→His, Tyr 132→Phe, and Lys 134→Val; and
Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp;
(m) Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val; and
Leu 36→Trp, Ala 40→Val, Ile 41→Lys, Asp 47→Gln, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp;
(n) Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val; and
Ala 40→Tyr, Ile 41→Arg, Gln 49→Ser, Tyr 52→Gly, Ser 68→Gln, Leu 70→Gln, Arg 72→Asp, Lys 73→Asp, Asp 77→Leu, Trp 79→Val, Arg 81→Glu, Asn 96→Ala, Tyr 106→Gln, Phe 123→Val, Ser 127→Trp, Gln 128→Pro, Arg 130→Leu, and Lys 134→Gln, Thr 136→Val; or
(o) Asp 47→Gln, Gln 49→Ala, Tyr 52→Phe, Leu 70→Arg, Arg 72→Glu, Lys 73→Ala, Lys 75→Ser, Asp 77→Lys, Trp 79→Thr, Arg 81→Lys, Leu 94→Ala, Asn 96→Tyr, Tyr 100→Ala, Leu 103→Gln, Tyr 106→Thr, Lys 125→His, Ser 127→Thr, Tyr 132→Ile, and Lys 134→Val; and
Leu 36→Trp, Ala 40→Val, Ile 41→Lys, Asp 47→Gln, Gln 49→Leu, Tyr 52→Ser, Ser 68→Glu, Leu 70→Val, Arg 72→Glu, Lys 73→Gln, Asp 77→His, Trp 79→Ile, Arg 81→Lys, Leu 94→Ala, Asn 96→Asp, Tyr 100→Pro, Leu 103→Tyr, Tyr 106→Ser, Tyr 132→Phe, and Lys 134→Trp.

23. The fusion protein of claim 21 or 22, wherein the fusion protein comprises two lipocalin muteins that comprise amino acid sequences that have at least 80%, at least 85%, at least 90%, at least 95%, at least 98%, or at least 99% sequence identity to the following amino acid sequences or that comprises the following amino acid sequences, respectively:
(a) the amino acid sequence shown in SEQ ID NO: 6 and SEQ ID NO: 31;
(b) the amino acid sequence shown in SEQ ID NO: 6 and SEQ ID NO: 15;
(c) the amino acid sequence shown in SEQ ID NO: 28 and SEQ ID NO: 15;
(d) the amino acid sequence shown in SEQ ID NO: 4 and SEQ ID NO: 9;
(e) the amino acid sequence shown in SEQ ID NO: 9 and SEQ ID NO: 4;
(f) the amino acid sequence shown in SEQ ID NO: 9 and SEQ ID NO: 30;
(g) the amino acid sequence shown in SEQ ID NO: 11 and SEQ ID NO: 31;
(h) the amino acid sequence shown in SEQ ID NO: 11 and SEQ ID NO: 28;
(i) the amino acid sequence shown in SEQ ID NO: 31 and SEQ ID NO: 15;
(j) the amino acid sequence shown in SEQ ID NO: 12 and SEQ ID NO: 31;
(k) the amino acid sequence shown in SEQ ID NO: 12 and SEQ ID NO: 28;
(l) the amino acid sequence shown in SEQ ID NO: 13 and SEQ ID NO: 31;
(m) the amino acid sequence shown in SEQ ID NO: 15 and SEQ ID NO: 31;
(n) the amino acid sequence shown in SEQ ID NO: 15 and SEQ ID NO: 28; or
(o) the amino acid sequence shown in SEQ ID NO: 15 and SEQ ID NO: 35.

24. The fusion protein of any one of claims 21-23, wherein the the fusion protein has at least 75%, at least 80%, at least 85%, preferably at least 90%, preferably at least 95%, preferably at least 98%, preferably at least 99% identity to an amino acid sequence selected from the group consisting of SEQ ID NOs: 62-76.

25. The fusion protein of any one of claims 21-24, wherein the fusion protein comprises an amino acid sequence selected from the group consisting of SEQ ID NOs: 62-76 or a fragment or variant thereof.

26. A nucleic acid molecule comprising a nucleotide sequence encoding a lipocalin mutein of any one of claims 1-20 or a fusion protein of any one of claims 21-25.

27. A vector comprising the nucleic acid molecule of claim 26.

28. A host cell containing a nucleic acid molecule of claim 26 or the vector of claim 27.

29. A method of producing a lipocalin mutein of any one of claims 1-20 or a fusion protein of any one of claims 21-25, wherein the mutein or the fusion protein is produced starting from the nucleic acid coding for the mutein or the fusion protein.

30. A method of binding and/or detecting CTGF in a subject, comprising applying a lipocalin mutein of any one of claims 1-20 or a fusion protein of any one of claims 21-25 or a composition comprising such mutein or fusion protein.

31. A method of providing an anti-fibrotic effect in a subject, comprising applying a lipocalin mutein of any one of claims 1-20 or a fusion protein of any one of claims 21-25 or a composition comprising such mutein or fusion protein.

32. A method of regulating downstream signaling pathways of CTGF, comprising applying a lipocalin mutein of any one of claims 1-20 or a fusion protein of any one of claims 21-25 or a composition comprising such mutein or fusion protein.

33. A method of reducing collagen deposition in the lung, comprising applying a lipocalin mutein of any one of claims 1-20 or a fusion protein of any one of claims 21-25 or a composition comprising such mutein or fusion protein.

34. A pharmaceutical composition comprising a lipocalin mutein of any one of claims 1-20 or a fusion protein of any one of claims 21-25.

35. A lipocalin mutein of any one of claims 1-20, a fusion protein of any one of claims 21-25, or the pharmaceutical composition of claim 34 for use in therapy.

36. The lipocalin mutein, fusion protein, or pharmaceutical composition for the use of claim 35, wherein the use is in the treatment of a fibrotic disease, a cancer, an autoimmune disease, or an infectious disease.

37. The lipocalin mutein, fusion protein, or pharmaceutical composition for the use of claim 35 or 36, wherein the use is in the treatment of a lung disease, a muscle disease, a heart disease, a liver disease, a kidney disease, or an eye disease.

38. The lipocalin mutein, fusion protein, or pharmaceutical composition for the use of any one of claims 35-37, wherein the use is in the treatment of a fibrotic disease.

39. The lipocalin mutein, fusion protein, or pharmaceutical composition for the use of any one of claims 35-38, wherein the use is in the treatment of idiopathic pulmonary fibrosis (IPF).

40. The lipocalin mutein, fusion protein, or pharmaceutical composition for the use of any one of claims 35-38, wherein the use is in the treatment of COVID-19.

41. Use of a lipocalin mutein of any one of claims 1-20 or a fusion protein of any one of claims 21-25 for the manufacture of a medicament.

42. The use of claim 41, wherein the medicament is for the treatment of a fibrotic disease, a cancer, a lung disease, and/or COVID-19.

43. The use of claim 41 or 42, wherein the medicament is for the treatment of IPF.

44. A method of treating a disease comprising administering to a subject in need thereof an effective amount of a lipocalin mutein of any one of claims 1-20, a fusion protein of any one of claims 21-25, or a pharmaceutical composition of claim 34.

45. The method of claim 44, wherein the disease is a fibrotic disease, a cancer, a lung disease, and/or COVID-19.

46. The method of claim 44 or 45, wherein the disease is IPF.


SEQUENCE LISTING (84 sequences)

SEQ ID NO: 1 - protein, 178 aa, human
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 2 - protein, 178 aa, Artificial Sequence
  lipocalin mutein
QDSTSDLIPAPPLSKVPLQQNFQDNQFHGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 3 - protein, 178 aa, Artificial Sequence
  lipocalin mutein
QDSTSDLIPAPPLSKVPLQQNFQDNQFHGKWYVVGRAGNNRLREDKDPPKMWATIYELKEDKSYNVTHVHFMTKKCRYIIDTFVPGSQPGEFTLGAIKSGPGMTSPLVRIVSTNYNQHAMVFFKWVNQNREWFTITLYGRTKELTSELKENFIRFSKSLGLPENHIVFPVPIDQCIDG

SEQ ID NO: 4 - protein, 178 aa, Artificial Sequence
  lipocalin mutein
QDSTSDLIPAPPLSKVPLQQNFQDNQFHGKWYVVGRAGNNRLREDKDPPKMWATIYELKEDKSYDVTHVHFMTKKCRYIIDTFVPGSQPGEFTLGAIKSGPGMTSPLVRIVSTNYNQHAMVFFKWVNQNREWFTITLYGRTKELTSELKENFIRFSKSLGLPENHIVFPVPIDQCIDG

SEQ ID NO: 5 - protein, 177 aa, Artificial Sequence
  lipocalin mutein
QDSTSDLIPAPPLSKVPLQQNFQDNQFQGKWYVVGKAGNALREDKEPPKMWATIYELKEDKSYDVTHVHFLTKKCKYLIDTFVPGSQPGEFTIGAIKSGPGLTSPLVRVVSTNYNQHAMVFFKWVSQNREWFTITLYGRTKELTSELKENFIRFSKSLGLPENHIVFPVPIDQCIDG

SEQ ID NO: 6 - protein, 178 aa, Artificial Sequence
  lipocalin mutein
QDSTSDLIPAPPLSKVPLQQNFQDNQFQGKWYVVGRAGNNQLREDKSPPKMWATIYELKEDKSYDVTHVHFSTKKCRYIIDTFVPGSQPGEFTAGAIKSGPGLTSPLVRVVSTNYNQHAMVFFKWVTQNREWFTITLYGRTKELTSELKENFIRFSKSLGLPENHIVFPVPIDQCIDG

SEQ ID NO: 7 - protein, 178 aa, Artificial Sequence
  lipocalin mutein
QDSTSDLIPAPPLSKVPLQQNFQDNQFHGKWYVVGLAGNYGLRTDKRPSKMFATIYELKEDKSYDVTSVRFEQERCHYTIRTFVPGSQPGEFTTGSIKSRPGQTSSLVRVVSTNYNQHAMVFFKSVIQNREYFAITLYGRTKELTSELKENFIRFSKSLGLPENHIVFPVPIDQCIDG

SEQ ID NO: 8 - protein, 178 aa, Artificial Sequence
  lipocalin mutein
QDSTSDLIPAPPLSKVPLQQNFQDNQFQGKWYVVGLAGNYGLRTDKRPSKMFATIYELKEDKSYDVTSVRFEQERCHYTIRTFVPGSQPGEFTSGNIGNYPGSYSTLVRVVSTNYNQHAMVFFKSVIQNREYFAITLYGRTKELTSELKENFIRFSKSLGLPENHIVFPVPIDQCIDG

SEQ ID NO: 9 - protein, 178 aa, Artificial Sequence
  lipocalin mutein
QDSTSDLIPAPPLSKVPLQQNFQDNQFHGKWYVVGLAGNKILRIDKQPAKMFATIYELKEDKSYDVTSVRFEARSCSYTIKTFVPGSQPGEFTAGNIKSAPGQTSTLVRVVSTNYNQHAMVFFKHVSQNRETFVITLYGRTKELTSELKENFIRFSKSLGLPENHIVFPVPIDQCIDG

SEQ ID NO: 10 - protein, 178 aa, Artificial Sequence
  lipocalin mutein
QDSTSDLIPAPPLSKVPLQQNFQDNQFQGKWYVVGLAGNAILREDKQPAKMFATIYELKEDKSYDVTSVRFEAKSCSYTIKTFVPGSQPGEFTAGNIKSAPGQTSTLVRVVSTNYNQHAMVFWKSVAGTRESFNIALYGRTKELTSELKENFIRFSKSLGLPENHIVFPVPIDQCIDG

SEQ ID NO: 11 - protein, 178 aa, Artificial Sequence
  lipocalin mutein
QDSTSDLIPAPPLSKVPLQQNFQDNQFQGKWYVVGLAGNAILREDKQPAKMFATIYELKEDKSYDVTSVRFEAKSCSYTSKTFVPGSQPGEFTAGNIKSAPGQTSTLVRVVSTNYNQHAMVFFKHVSQNRETFVITLYGRTKELTSELKENFIRFSKSLGLPENHIVFPVPIDQCIDG

SEQ ID NO: 12 - protein, 178 aa, Artificial Sequence
  lipocalin mutein
QDSTSDLIPAPPLSKVPLQQNFQDNQFQGKWYVVGLAGNAILREDKYPAKMFATIYELKEDKSYDVTSVRFENKSCVYTIKTFVPGSQPGEFTTGNIKSAPGQTSTLVRVVSTNYNQHAMVFFKHVSQNREFFVITLYGRTKELTSELKENFIRFSKSLGLPENHIVFPVPIDQCIDG

SEQ ID NO: 13 - protein, 178 aa, Artificial Sequence
  lipocalin mutein
QDSTSDLIPAPPLSKVPLQQNFQDNQFQGKWYVVGLAGNAILREDKYPAKMFATIYELKEDKSYDVTSVRFENKSCVYTSKTFVPGSQPGEFTTGNIKSAPGQTSTLVRVVSTNYNQHAMVFFKHVSQNREFFVITLYGRTKELTSELKENFIRFSKSLGLPENHIVFPVPIDQCIDG

SEQ ID NO: 14 - protein, 178 aa, Artificial Sequence
  lipocalin mutein
QDSTSDLIPAPPLSKVPLQQNFQDNQFQGKWYVVGLAGNAILREDKYPAKMFATIYELKEDKSYDVTSVRFEAKSCIYTIKTFVPGSQPGEFTRGNIKSAPGQTSTLVRVVSTNYNQHAMVFFKHVSQNREIFVITLYGRTKELTSELKENFIRFSKSLGLPENHIVFPVPIDQCIDG

SEQ ID NO: 15 - protein, 178 aa, Artificial Sequence
  lipocalin mutein
QDSTSDLIPAPPLSKVPLQQNFQDNQFQGKWYVVGLAGNAILREDKQPAKMFATIYELKEDKSYDVTSVRFEAKSCKYTIKTFVPGSQPGEFTAGYIKSAPGQTSTLVRVVSTNYNQHAMVFFKHVTQNREIFVITLYGRTKELTSELKENFIRFSKSLGLPENHIVFPVPIDQCIDG

SEQ ID NO: 16 - protein, 178 aa, Artificial Sequence
  lipocalin mutein
QDSTSDLIPAPPLSKVPLQQNFQDNQFQGKWYVVGLAGNAILRTDKQPAKMFATIYELKEDKSYDVTSVRFEAKSCVYTIKTFVPGSQPGEFTAGNIKSAPGQTSTLVRVVSTNYNQHAMVFFKHVSQNREFFVITLYGRTKELTSELKENFIRFSKSLGLPENHIVFPVPIDQCIDG

SEQ ID NO: 17 - protein, 178 aa, Artificial Sequence
  lipocalin mutein
QDSTSDLIPAPPLSKVPLQQNFQDNQFQGKWYVVGLAGNAILRTDKQPAKMFATIYELKEDKSYDVTSVRFEAKSCVYTSKTFVPGSQPGEFTAGNIKSAPGQTSTLVRVVSTNYNQHAMVFFKHVSQNREFFVITLYGRTKELTSELKENFIRFSKSLGLPENHIVFPVPIDQCIDG

SEQ ID NO: 18 - protein, 178 aa, Artificial Sequence
  lipocalin mutein
QDSTSDLIPAPPLSKVPLQQNFQDNQFQGKWYVVGIAGNAILRDDKYPAKMFATIYELKEDKSYDVTSVRFENKSCIYTIKTFVPGSQPGEFTHGNIKSHPGQTSTLVRVVSTNYNQHAMVFFKHVSQNREIFVITLYGRTKELTSELKENFIRFSKSLGLPENHIVFPVPIDQCIDG

SEQ ID NO: 19 - protein, 178 aa, Artificial Sequence
  lipocalin mutein
QDSTSDLIPAPPLSKVPLQQNFQDNQFQGKWYVVGLAGNAILREDKQPAKMFATIYELKEDKSYDVTSVRFEAKSCSYTIKTFVPGSQPGEFTAGNIKSAPGQTSTLVRVVSTNYNQHAMVFHKAVSQNRESFVITLYGRTKELTSELKENFIRFSKSLGLPENHIVFPVPIDQCIDG

SEQ ID NO: 20 - protein, 178 aa, Artificial Sequence
  lipocalin mutein
QDSTSDLIPAPPLSKVPLQQNFQDNQFQGKWYVVGLAGNAILREDKQPAKMFATIYELKEDKSYDVTSVRFEAKSCSYTIKTFVPGSQPGEFTAGNIKSAPGQTSTLVRVVSTNYNQHAMVFAKAVQLNREHFFITLYGRTKELTSELKENFIRFSKSLGLPENHIVFPVPIDQCIDG

SEQ ID NO: 21 - protein, 178 aa, Artificial Sequence
  lipocalin mutein
QDSTSDLIPAPPLSKVPLQQNFQDNQFQGKWYVVGLAGNAILREDKQPAKMFATIYELKEDKSYDVTSVRFEAKSCSYTIKTFVPGSQPGEFTAGNIKSAPGQTSTLVRVVSTNYNQHAMVFFKAVRGARESFNIALYGRTKELTSELKENFIRFSKSLGLPENHIVFPVPIDQCIDG

SEQ ID NO: 22 - protein, 178 aa, Artificial Sequence
  lipocalin mutein
QDSTSDLIPAPPLSKVPLQQNFQDNQFQGKWYVVGIAGNAILRVDKQPAKMFATIYELKEDKSYDVTSVRFEAKSCVYTIKTFVPGSQPGEFTAGNIKSAPGQTSTLVRVVSTNYNQHAMVFFKHVSQNREIFVITLYGRTKELTSELKENFIRFSKSLGLPENHIVFPVPIDQCIDG

SEQ ID NO: 23 - protein, 178 aa, Artificial Sequence
  lipocalin mutein
QDSTSDLIPAPPLSKVPLQQNFQDNQFQGKWYVVGVAGNAILRPDKQPAKMFATIYELKEDKSYDVTSVRFEAKSCKYTIKTFVPGSQPGEFTSGQIKSAPGQTSTLVRVVSTNYNQHAMVFFKHVSQNREIFVITLYGRTKELTSELKENFIRFSKSLGLPENHIVFPVPIDQCIDG

SEQ ID NO: 24 - protein, 178 aa, Artificial Sequence
  lipocalin mutein
QDSTSDLIPAPPLSKVPLQQNFQDNQFQGKWYVVGVAGNAILRTDKQPAKMFATIYELKEDKSYDVTSVRFEAKSCKYTIKTFVPGSQPGEFTAGNIKSAPGQTSTLVRVVSTNYNQHAMVFFKHVSQNREIFVITLYGRTKELTSELKENFIRFSKSLGLPENHIVFPVPIDQCIDG

SEQ ID NO: 25 - protein, 178 aa, Artificial Sequence
  lipocalin mutein
QDSTSDLIPAPPLSKVPLQQNFQDNQFHGKWYVVGMAGNFRLREDKDPSKMGATIYELKEDKSYNVTQVQFDDKKCLYVIETFVPGSQPGEFTLGAIKSFPGFTSQLVRVVSTNYNQHAMVFFKKVYQNREVFWITLYGRTKELTSELKENFIRFSKSLGLPENHIVFPVPIDQCIDG

SEQ ID NO: 26 - protein, 178 aa, Artificial Sequence
  lipocalin mutein
QDSTSDLIPAPPLSKVPLQQNFQDNQFQGKWYVVGLAGNYRLREDKDPSKMGATIYELKEDKSYDVTQVQFDDKKCLYVIETFVPGSQPGEFTLGNIKVYPTLESQLVRVVSTNYNQHAMVFFKKVYQNREVFWITLYGRTKELTSELKENFIRFSKSLGLPENHIVFPVPIDQCIDG

SEQ ID NO: 27 - protein, 178 aa, Artificial Sequence
  lipocalin mutein
QDSTSDLIPAPPLSKVPLQQNFQDNQFQGKWYVVGLAGNYRLREDKDPSKMGATIYELKEDKSYDVTQVQFDDKKCLYVIETFVPGSQPGEFTLGAIKSYPGLTSQLVRVVSTNYNQHAMVFLKKVYGSEEYFHITLYGRTKELTSELKENFIRFSKSLGLPENHIVFPVPIDQCIDG

SEQ ID NO: 28 - protein, 178 aa, Artificial Sequence
  lipocalin mutein
QDSTSDLIPAPPLSKVPLQQNFQDNQFQGKWYVVGLAGNYRLREDKDPSKMGATIYELKEDKSYDVTQVQFDDKKCLYVIETFVPGSQPGEFTLGAIKSYPGLTSQLVRVVSTNYNQHAMVFVKKVWPNLEYFQIVLYGRTKELTSELKENFIRFSKSLGLPENHIVFPVPIDQCIDG

SEQ ID NO: 29 - protein, 178 aa, Artificial Sequence
  lipocalin mutein
QDSTSDLIPAPPLSKVPLQQNFQDNQFHGKWYVVGWAGNIKLREDKDPFKMSATIYELKEDKSYNVTEVVFEQKKCHYIIKTFVPGSQPGEFTLGDIKSPPGETSSLVRVVSTNYNQHAMVFFKKVFQNREFFWITLYGRTKELTSELKENFIRFSKSLGLPENHIVFPVPIDQCIDG

SEQ ID NO: 30 - protein, 178 aa, Artificial Sequence
  lipocalin mutein
QDSTSDLIPAPPLSKVPLQQNFQDNQFHGKWYVVGWAGNIKLREDKDPFKMSATIYELKEDKSYDVTEVVFEQKKCHYIIKTFVPGSQPGEFTLGDIKSPPGETSSLVRVVSTNYNQHAMVFFKKVFQNREFFWITLYGRTKELTSELKENFIRFSKSLGLPENHIVFPVPIDQCIDG

SEQ ID NO: 31 - protein, 178 aa, Artificial Sequence
  lipocalin mutein
QDSTSDLIPAPPLSKVPLQQNFQDNQFQGKWYVVGWAGNVKLREDKQPQKMSATIYELKEDKSYDVTEVVFEQKKCHYIIKTFVPGSQPGEFTAGDIKSPPGYTSSLVRVVSTNYNQHAMVFFKKVSQNREFFWITLYGRTKELTSELKENFIRFSKSLGLPENHIVFPVPIDQCIDG

SEQ ID NO: 32 - protein, 178 aa, Artificial Sequence
  lipocalin mutein
QDSTSDLIPAPPLSKVPLQQNFQDNQFQGKWYVVGWAGNVKLREDKQPFKMSATIYELKEDKSYDVTEVVFEQKKCHYIIKTFVPGSQPGEFTLSPISSSPGLVSHLVRVVSTNYNQHAMVFFKKVHQNREFFWITLYGRTKELTSELKENFIRFSKSLGLPENHIVFPVPIDQCIDG

SEQ ID NO: 33 - protein, 178 aa, Artificial Sequence
  lipocalin mutein
QDSTSDLIPAPPLSKVPLQQNFQDNQFQGKWYVVGWAGNAKLREDKQPFKMSATIYELKEDKSYDVTEVVFEQKKCHYIIKTFVPGSQPGEFTEGNYKRRPRLWSDLVRVVSTNYNQHAMVFFKKVHQNREFFWITLYGRTKELTSELKENFIRFSKSLGLPENHIVFPVPIDQCIDG

SEQ ID NO: 34 - protein, 178 aa, Artificial Sequence
  lipocalin mutein
QDSTSDLIPAPPLSKVPLQQNFQDNQFQGKWYVVGWAGNVILREDKQPFKMSATIYELKEDKSYDVTEVVFEQKKCHYIIKTFVPGSQPGEFTLGDIKSPPGETSTLVRVVSTNYNQHAMVFFKKVSQNREFFWITLYGRTKELTSELKENFIRFSKSLGLPENHIVFPVPIDQCIDG

SEQ ID NO: 35 - protein, 178 aa, Artificial Sequence
  lipocalin mutein
QDSTSDLIPAPPLSKVPLQQNFQDNQFQGKWYVVGWAGNVKLREDKQPLKMSATIYELKEDKSYDVTEVVFEQKKCHYIIKTFVPGSQPGEFTAGDIKSPPGYTSSLVRVVSTNYNQHAMVFFKKVSQNREFFWITLYGRTKELTSELKENFIRFSKSLGLPENHIVFPVPIDQCIDG

SEQ ID NO: 36 - protein, 178 aa, Artificial Sequence
  lipocalin mutein
QDSTSDLIPAPPLSKVPLQQNFQDNQFQGKWYVVGWAGNVKLREDKQPAKMSATIYELKEDKSYDVTEVVFEQKKCHYIIKTFVPGSQPGEFTAGDIKSPPGYTSSLVRVVSTNYNQHAMVFFKKVGQNREFFWITLYGRTKELTSELKENFIRFSKSLGLPENHIVFPVPIDQCIDG

SEQ ID NO: 37 - protein, 10 aa, Artificial Sequence
  His tag
HHHHHHHHHH

SEQ ID NO: 38 - protein, 10 aa, Artificial Sequence
  Myc tag
EQKLISEEDL

SEQ ID NO: 39 - protein, 4 aa, Artificial Sequence
  Factor Xa cleavage site
IEGR

SEQ ID NO: 40 - protein, 10 aa, Artificial Sequence
  fusion peptide
SAWSHPQFEK

SEQ ID NO: 41 - protein, 8 aa, Artificial Sequence
  Strep-tag II peptide
WSHPQFEK

SEQ ID NO: 42 - protein, 15 aa, Artificial Sequence
  linker
GGGGSGGGGSGGGGS

SEQ ID NO: 43 - protein, 18 aa, Artificial Sequence
  linker
PSTPPTNSSSTPPTPSPS

SEQ ID NO: 44 - protein, 14 aa, Artificial Sequence
  linker
GGSGNSSGSGGSPV

SEQ ID NO: 45 - protein, 20 aa, Artificial Sequence
  linker
ASPAAPAPASPAAPAPSAPA

SEQ ID NO: 46 - protein, 66 aa, Artificial Sequence
  linker
AGSGGSGGSGGSPVPSTPPTPSPSTPPTPSPSGGSGNSSGSGGSPVPSTPPTPSPSTPPTPSPSAS

SEQ ID NO: 47 - protein, 32 aa, Artificial Sequence
  linker
PSTPPTPSPSTPPTPSPSGGSGNSSGSGGSPV

SEQ ID NO: 48 - protein, 74 aa, Artificial Sequence
  linker
AGSGGSGGSGGSPVPSTPPTNSSSTPPTPSPSPVPSTPPTNSSSTPPTPSPSPVPSTPPTNSSSTPPTPSPSAS

SEQ ID NO: 49 - protein, 40 aa, Artificial Sequence
  linker
ASPAAPAPASPAAPAPSAPAASPAAPAPASPAAPAPSAPA

SEQ ID NO: 50 - protein, 10 aa, Artificial Sequence
  linker
VDDIEGRMDE

SEQ ID NO: 51 - protein, 11 aa, Artificial Sequence
  linker
ENLYFQGRMDE

SEQ ID NO: 52 - protein, 10 aa, Artificial Sequence
  linker
GGGGSGGGGS

SEQ ID NO: 53 - protein, 8 aa, Artificial Sequence
  antibody heavy chain CDR1 sequence
GFTFSSYG

SEQ ID NO: 54 - protein, 7 aa, Artificial Sequence
  antibody heavy chain CDR2 sequence
IGTGGGT

SEQ ID NO: 55 - protein, 14 aa, Artificial Sequence
  antibody heavy chain CDR3 sequence
ARGDYYGSGSFFDC

SEQ ID NO: 56 - protein, 6 aa, Artificial Sequence
  antibody light chain CDR1 sequence
QGISSW

SEQ ID NO: 57 - protein, 9 aa, Artificial Sequence
  antibody light chain CDR3 sequence
QQYNSYPPT

SEQ ID NO: 58 - protein, 120 aa, Artificial Sequence
  antibody heavy chain variable region
EGQLVQSGGGLVHPGGSLRLSCAGSGFTFSSYGMHWVRQAPGKGLEWVSGIGTGGGTYSTDSVKGRFTISRDNAKNSLYLQMNSLRAEDMAVYYCARGDYYGSGSFFDCWGQGTLVTVSS

SEQ ID NO: 59 - protein, 107 aa, Artificial Sequence
  antibody light chain variable region
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIK

SEQ ID NO: 60 - protein, 450 aa, Artificial Sequence
  antibody heavy chain
EGQLVQSGGGLVHPGGSLRLSCAGSGFTFSSYGMHWVRQAPGKGLEWVSGIGTGGGTYSTDSVKGRFTISRDNAKNSLYLQMNSLRAEDMAVYYCARGDYYGSGSFFDCWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 214 aa, Artificial Sequence
  antibody light chain
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 371 aa, Artificial Sequence
  fusion protein
QDSTSDLIPAPPLSKVPLQQNFQDNQFQGKWYVVGRAGNNQLREDKSPPKMWATIYELKEDKSYDVTHVHFSTKKCRYIIDTFVPGSQPGEFTAGAIKSGPGLTSPLVRVVSTNYNQHAMVFFKWVTQNREWFTITLYGRTKELTSELKENFIRFSKSLGLPENHIVFPVPIDQCIDGGGGGSGGGGSGGGGSQDSTSDLIPAPPLSKVPLQQNFQDNQFQGKWYVVGWAGNVKLREDKQPQKMSATIYELKEDKSYDVTEVVFEQKKCHYIIKTFVPGSQPGEFTAGDIKSPPGYTSSLVRVVSTNYNQHAMVFFKKVSQNREFFWITLYGRTKELTSELKENFIRFSKSLGLPENHIVFPVPIDQCIDG

SEQ ID NO: 63 - protein, 371 aa, Artificial Sequence
  fusion protein
QDSTSDLIPAPPLSKVPLQQNFQDNQFQGKWYVVGRAGNNQLREDKSPPKMWATIYELKEDKSYDVTHVHFSTKKCRYIIDTFVPGSQPGEFTAGAIKSGPGLTSPLVRVVSTNYNQHAMVFFKWVTQNREWFTITLYGRTKELTSELKENFIRFSKSLGLPENHIVFPVPIDQCIDGGGGGSGGGGSGGGGSQDSTSDLIPAPPLSKVPLQQNFQDNQFQGKWYVVGLAGNAILREDKQPAKMFATIYELKEDKSYDVTSVRFEAKSCKYTIKTFVPGSQPGEFTAGYIKSAPGQTSTLVRVVSTNYNQHAMVFFKHVTQNREIFVITLYGRTKELTSELKENFIRFSKSLGLPENHIVFPVPIDQCIDG

SEQ ID NO: 64 - protein, 371 aa, Artificial Sequence
  fusion protein
QDSTSDLIPAPPLSKVPLQQNFQDNQFQGKWYVVGLAGNYRLREDKDPSKMGATIYELKEDKSYDVTQVQFDDKKCLYVIETFVPGSQPGEFTLGAIKSYPGLTSQLVRVVSTNYNQHAMVFVKKVWPNLEYFQIVLYGRTKELTSELKENFIRFSKSLGLPENHIVFPVPIDQCIDGGGGGSGGGGSGGGGSQDSTSDLIPAPPLSKVPLQQNFQDNQFQGKWYVVGLAGNAILREDKQPAKMFATIYELKEDKSYDVTSVRFEAKSCKYTIKTFVPGSQPGEFTAGYIKSAPGQTSTLVRVVSTNYNQHAMVFFKHVTQNREIFVITLYGRTKELTSELKENFIRFSKSLGLPENHIVFPVPIDQCIDG

SEQ ID NO: 65 - protein, 371 aa, Artificial Sequence
  fusion protein
QDSTSDLIPAPPLSKVPLQQNFQDNQFHGKWYVVGRAGNNRLREDKDPPKMWATIYELKEDKSYDVTHVHFMTKKCRYIIDTFVPGSQPGEFTLGAIKSGPGMTSPLVRIVSTNYNQHAMVFFKWVNQNREWFTITLYGRTKELTSELKENFIRFSKSLGLPENHIVFPVPIDQCIDGGGGGSGGGGSGGGGSQDSTSDLIPAPPLSKVPLQQNFQDNQFHGKWYVVGLAGNKILRIDKQPAKMFATIYELKEDKSYDVTSVRFEARSCSYTIKTFVPGSQPGEFTAGNIKSAPGQTSTLVRVVSTNYNQHAMVFFKHVSQNRETFVITLYGRTKELTSELKENFIRFSKSLGLPENHIVFPVPIDQCIDG

SEQ ID NO: 66 - protein, 371 aa, Artificial Sequence
  fusion protein
QDSTSDLIPAPPLSKVPLQQNFQDNQFHGKWYVVGLAGNKILRIDKQPAKMFATIYELKEDKSYDVTSVRFEARSCSYTIKTFVPGSQPGEFTAGNIKSAPGQTSTLVRVVSTNYNQHAMVFFKHVSQNRETFVITLYGRTKELTSELKENFIRFSKSLGLPENHIVFPVPIDQCIDGGGGGSGGGGSGGGGSQDSTSDLIPAPPLSKVPLQQNFQDNQFHGKWYVVGRAGNNRLREDKDPPKMWATIYELKEDKSYDVTHVHFMTKKCRYIIDTFVPGSQPGEFTLGAIKSGPGMTSPLVRIVSTNYNQHAMVFFKWVNQNREWFTITLYGRTKELTSELKENFIRFSKSLGLPENHIVFPVPIDQCIDG

SEQ ID NO: 67 - protein, 371 aa, Artificial Sequence
  fusion protein
QDSTSDLIPAPPLSKVPLQQNFQDNQFHGKWYVVGLAGNKILRIDKQPAKMFATIYELKEDKSYDVTSVRFEARSCSYTIKTFVPGSQPGEFTAGNIKSAPGQTSTLVRVVSTNYNQHAMVFFKHVSQNRETFVITLYGRTKELTSELKENFIRFSKSLGLPENHIVFPVPIDQCIDGGGGGSGGGGSGGGGSQDSTSDLIPAPPLSKVPLQQNFQDNQFHGKWYVVGWAGNIKLREDKDPFKMSATIYELKEDKSYDVTEVVFEQKKCHYIIKTFVPGSQPGEFTLGDIKSPPGETSSLVRVVSTNYNQHAMVFFKKVFQNREFFWITLYGRTKELTSELKENFIRFSKSLGLPENHIVFPVPIDQCIDG

SEQ ID NO: 68 - protein, 371 aa, Artificial Sequence
  fusion protein
QDSTSDLIPAPPLSKVPLQQNFQDNQFQGKWYVVGLAGNAILREDKQPAKMFATIYELKEDKSYDVTSVRFEAKSCSYTSKTFVPGSQPGEFTAGNIKSAPGQTSTLVRVVSTNYNQHAMVFFKHVSQNRETFVITLYGRTKELTSELKENFIRFSKSLGLPENHIVFPVPIDQCIDGGGGGSGGGGSGGGGSQDSTSDLIPAPPLSKVPLQQNFQDNQFQGKWYVVGWAGNVKLREDKQPQKMSATIYELKEDKSYDVTEVVFEQKKCHYIIKTFVPGSQPGEFTAGDIKSPPGYTSSLVRVVSTNYNQHAMVFFKKVSQNREFFWITLYGRTKELTSELKENFIRFSKSLGLPENHIVFPVPIDQCIDG

SEQ ID NO: 69 - protein, 371 aa, Artificial Sequence
  fusion protein
QDSTSDLIPAPPLSKVPLQQNFQDNQFQGKWYVVGLAGNAILREDKQPAKMFATIYELKEDKSYDVTSVRFEAKSCSYTSKTFVPGSQPGEFTAGNIKSAPGQTSTLVRVVSTNYNQHAMVFFKHVSQNRETFVITLYGRTKELTSELKENFIRFSKSLGLPENHIVFPVPIDQCIDGGGGGSGGGGSGGGGSQDSTSDLIPAPPLSKVPLQQNFQDNQFQGKWYVVGLAGNYRLREDKDPSKMGATIYELKEDKSYDVTQVQFDDKKCLYVIETFVPGSQPGEFTLGAIKSYPGLTSQLVRVVSTNYNQHAMVFVKKVWPNLEYFQIVLYGRTKELTSELKENFIRFSKSLGLPENHIVFPVPIDQCIDG

SEQ ID NO: 70 - protein, 371 aa, Artificial Sequence
  fusion protein
QDSTSDLIPAPPLSKVPLQQNFQDNQFQGKWYVVGWAGNVKLREDKQPQKMSATIYELKEDKSYDVTEVVFEQKKCHYIIKTFVPGSQPGEFTAGDIKSPPGYTSSLVRVVSTNYNQHAMVFFKKVSQNREFFWITLYGRTKELTSELKENFIRFSKSLGLPENHIVFPVPIDQCIDGGGGGSGGGGSGGGGSQDSTSDLIPAPPLSKVPLQQNFQDNQFQGKWYVVGLAGNAILREDKQPAKMFATIYELKEDKSYDVTSVRFEAKSCKYTIKTFVPGSQPGEFTAGYIKSAPGQTSTLVRVVSTNYNQHAMVFFKHVTQNREIFVITLYGRTKELTSELKENFIRFSKSLGLPENHIVFPVPIDQCIDG

SEQ ID NO: 71 - protein, 371 aa, Artificial Sequence
  fusion protein
QDSTSDLIPAPPLSKVPLQQNFQDNQFQGKWYVVGLAGNAILREDKYPAKMFATIYELKEDKSYDVTSVRFENKSCVYTIKTFVPGSQPGEFTTGNIKSAPGQTSTLVRVVSTNYNQHAMVFFKHVSQNREFFVITLYGRTKELTSELKENFIRFSKSLGLPENHIVFPVPIDQCIDGGGGGSGGGGSGGGGSQDSTSDLIPAPPLSKVPLQQNFQDNQFQGKWYVVGWAGNVKLREDKQPQKMSATIYELKEDKSYDVTEVVFEQKKCHYIIKTFVPGSQPGEFTAGDIKSPPGYTSSLVRVVSTNYNQHAMVFFKKVSQNREFFWITLYGRTKELTSELKENFIRFSKSLGLPENHIVFPVPIDQCIDG

SEQ ID NO: 72 - protein, 371 aa, Artificial Sequence
  fusion protein
QDSTSDLIPAPPLSKVPLQQNFQDNQFQGKWYVVGLAGNAILREDKYPAKMFATIYELKEDKSYDVTSVRFENKSCVYTIKTFVPGSQPGEFTTGNIKSAPGQTSTLVRVVSTNYNQHAMVFFKHVSQNREFFVITLYGRTKELTSELKENFIRFSKSLGLPENHIVFPVPIDQCIDGGGGGSGGGGSGGGGSQDSTSDLIPAPPLSKVPLQQNFQDNQFQGKWYVVGLAGNYRLREDKDPSKMGATIYELKEDKSYDVTQVQFDDKKCLYVIETFVPGSQPGEFTLGAIKSYPGLTSQLVRVVSTNYNQHAMVFVKKVWPNLEYFQIVLYGRTKELTSELKENFIRFSKSLGLPENHIVFPVPIDQCIDG

SEQ ID NO: 73 - protein, 371 aa, Artificial Sequence
  fusion protein
QDSTSDLIPAPPLSKVPLQQNFQDNQFQGKWYVVGLAGNAILREDKYPAKMFATIYELKEDKSYDVTSVRFENKSCVYTSKTFVPGSQPGEFTTGNIKSAPGQTSTLVRVVSTNYNQHAMVFFKHVSQNREFFVITLYGRTKELTSELKENFIRFSKSLGLPENHIVFPVPIDQCIDGGGGGSGGGGSGGGGSQDSTSDLIPAPPLSKVPLQQNFQDNQFQGKWYVVGWAGNVKLREDKQPQKMSATIYELKEDKSYDVTEVVFEQKKCHYIIKTFVPGSQPGEFTAGDIKSPPGYTSSLVRVVSTNYNQHAMVFFKKVSQNREFFWITLYGRTKELTSELKENFIRFSKSLGLPENHIVFPVPIDQCIDG

SEQ ID NO: 74 - protein, 371 aa, Artificial Sequence
  fusion protein
QDSTSDLIPAPPLSKVPLQQNFQDNQFQGKWYVVGLAGNAILREDKQPAKMFATIYELKEDKSYDVTSVRFEAKSCKYTIKTFVPGSQPGEFTAGYIKSAPGQTSTLVRVVSTNYNQHAMVFFKHVTQNREIFVITLYGRTKELTSELKENFIRFSKSLGLPENHIVFPVPIDQCIDGGGGGSGGGGSGGGGSQDSTSDLIPAPPLSKVPLQQNFQDNQFQGKWYVVGWAGNVKLREDKQPQKMSATIYELKEDKSYDVTEVVFEQKKCHYIIKTFVPGSQPGEFTAGDIKSPPGYTSSLVRVVSTNYNQHAMVFFKKVSQNREFFWITLYGRTKELTSELKENFIRFSKSLGLPENHIVFPVPIDQCIDG

SEQ ID NO: 75 - protein, 371 aa, Artificial Sequence
  fusion protein
QDSTSDLIPAPPLSKVPLQQNFQDNQFQGKWYVVGLAGNAILREDKQPAKMFATIYELKEDKSYDVTSVRFEAKSCKYTIKTFVPGSQPGEFTAGYIKSAPGQTSTLVRVVSTNYNQHAMVFFKHVTQNREIFVITLYGRTKELTSELKENFIRFSKSLGLPENHIVFPVPIDQCIDGGGGGSGGGGSGGGGSQDSTSDLIPAPPLSKVPLQQNFQDNQFQGKWYVVGLAGNYRLREDKDPSKMGATIYELKEDKSYDVTQVQFDDKKCLYVIETFVPGSQPGEFTLGAIKSYPGLTSQLVRVVSTNYNQHAMVFVKKVWPNLEYFQIVLYGRTKELTSELKENFIRFSKSLGLPENHIVFPVPIDQCIDG

SEQ ID NO: 76 - protein, 371 aa, Artificial Sequence
  fusion protein
QDSTSDLIPAPPLSKVPLQQNFQDNQFQGKWYVVGLAGNAILREDKQPAKMFATIYELKEDKSYDVTSVRFEAKSCKYTIKTFVPGSQPGEFTAGYIKSAPGQTSTLVRVVSTNYNQHAMVFFKHVTQNREIFVITLYGRTKELTSELKENFIRFSKSLGLPENHIVFPVPIDQCIDGGGGGSGGGGSGGGGSQDSTSDLIPAPPLSKVPLQQNFQDNQFQGKWYVVGWAGNVKLREDKQPLKMSATIYELKEDKSYDVTEVVFEQKKCHYIIKTFVPGSQPGEFTAGDIKSPPGYTSSLVRVVSTNYNQHAMVFFKKVSQNREFFWITLYGRTKELTSELKENFIRFSKSLGLPENHIVFPVPIDQCIDG

SEQ ID NO: 77 - protein, 385 aa, Artificial Sequence
  human CTGF domains 1 and 2 with Fc Tag
QNCSGPCRCPDEPAPRCPAGVSLVLDGCGCCRVCAKQLGELCTERDPCDPHKGLFCHFGSPANRKIGVCTAKDGAPCIFGGTVYRSGESFQSSCKYQCTCLDGAVGCMPLCSMDVRLPSPDCPFPRRVKLPGKCCEEWVCDEGGGGSENLYFQGRMDEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 385 aa, Artificial Sequence
  murine CTGF domains 1 and 2 with Fc Tag
QDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEGGGGSENLYFQGRMDEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 323 aa, Artificial Sequence
  human CTGF fragment
QNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 80 - protein, 565 aa, Artificial Sequence
  murine CTGF fragment with Fc tag
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSENLYFQGRMDEQDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTAVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMA

SEQ ID NO: 81 - protein, 565 aa, Artificial Sequence
  cynomolgus CTGF fragment with Fc tag
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSENLYFQGRMDEQNCSGPCRCPAEQAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSVKTYRAKFCGVCTDGRCCTPHRTTTLPVEFKCPDGEVMKKNMMFIKTCACHYNCPGDNDIFESLYYRKMYGDMA

SEQ ID NO: 82 - protein, 323 aa, Artificial Sequence
  rat CTGF fragment
QDCSAQCQCAAEAAPRCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTVVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMA

SEQ ID NO: 83 - protein, 564 aa, Artificial Sequence
  human CTGF fragment with Fc tag
QNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMAVDDIEGRMDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 539 aa, Artificial Sequence
  murine CTGF fragment with GST tag
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDQDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTAVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMY